(19) United States
(12) Patent Application Publication
(10) Pub. No.: US 2024/0197694 A1
(43) Pub. Date: Jun. 20, 2024

(54) MODIFIED PROTEINS AND PROTEIN BINDERS

(71) Applicant: Cullgen (Shanghai), Inc., Shanghai (CN)

(72) Inventors: Jing LIU, Oradell, NJ (US); Michael Bruno PLEWE, San Diego, CA (US); Matthew Randolph LEE, Del Mar, CA (US); Xiaoran HAN, Shanghai (CN); Liqun CHEN, Shanghai (CN); Ting YANG, Shanghai (CN); Chengwei ZHANG, Shanghai (CN); Jialiang WANG, Nashville, TN (US)

(21) Appl. No.: 18/550,345

(22) PCT Filed: Mar. 14, 2022
(86) PCT No.: PCT/CN2022/080634

§ 371 Date: Sep. 13, 2023

(30) Foreign Application Priority Data
Mar. 16, 2021 (WO) PCT/CN2021/081117

(51) Int. Cl. A61K 31/437; A61K 31/417; A61K 31/4725; C07D 233/64; C07D 401/12; C07D 471/04

(52) U.S. Cl. CPC A61K 31/437; A61K 31/417; A61K 31/4725; C07D 233/64; C07D 401/12; C07D 471/04

(57) ABSTRACT

Provided herein are compounds, pharmaceutical compositions, and methods for binding or modulating a DDB1- and CUL4-associated factor 1 (DCAF1) protein. Further provided herein are ligand-DCAF1 complexes or in vivo modified DCAF1 proteins.

### CROSS-REFERENCE

This application claims the benefit of PCT Application No. PCT/CN2021/081117, filed Mar. 16, 2021, which application is incorporated herein by reference in its entirety.

### BACKGROUND

A need exists for ligands for binding or modifying proteins. A need exists in the medicinal arts for selective modulation of proteins.

### SUMMARY

Described herein are modified proteins and protein-ligand complexes. The modified proteins and protein-ligand complexes of some embodiments are useful for biotechnology applications such as selective modulation of a protein.

Described herein are ligands that can bind to DDB1- and CUL4-associated factor 1 (DCAF1). The DCAF1 binding ligands are useful for biotechnology applications such as selective modulation of DCAF1.

In one aspect, provided herein is a compound of Formula Ia:

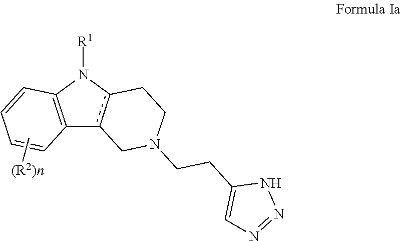

or a pharmaceutically acceptable salt thereof, wherein:
[symbol] is a single bond or a double bond;
R¹ is selected from H, C₁₋₁₀alkyl, C₂₋₁₀alkenyl, C₂₋₁₀alkynyl, C₁₋₁₀haloalkyl, and C₁₋₁₀heteroalkyl;
each R² is independently selected from H, halo, hydroxy, amino, cyano, nitro, C₁₋₈alkyl, C₂₋₈alkenyl, C₂₋₈alkynyl, C₁₋₈haloalkyl, C₁₋₈heteroalkyl, C₁₋₈alkoxy, C₃₋₁₀carbocyclyl, 3- to 10-membered heterocyclyl, C₆₋₁₀aryl, and 5- to 10-membered heteroaryl; and
n is 0, 1, 2, 3, or 4;
provided that the compound of Formula Ia is not

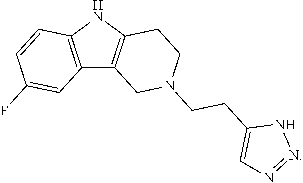 [SMILES: Fc1ccc2[nH]c3c(c2c1)CN(CCc1cnn[nH]1)CC3]

In another aspect, provided herein is a compound selected from:

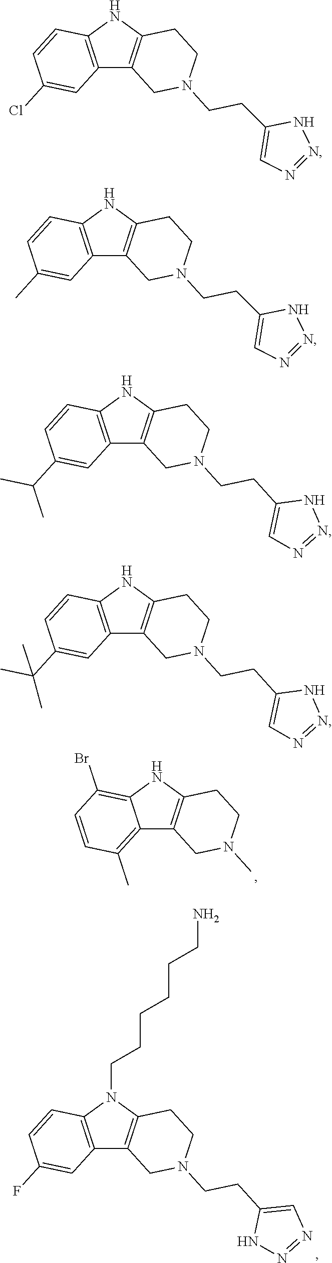 [SMILES: Clc1ccc2[nH]c3c(c2c1)CN(CCc1cnn[nH]1)CC3 ; Cc1ccc2[nH]c3c(c2c1)CN(CCc1cnn[nH]1)CC3 ; CC(C)c1ccc2[nH]c3c(c2c1)CN(CCc1cnn[nH]1)CC3 ; CC(C)(C)c1ccc2[nH]c3c(c2c1)CN(CCc1cnn[nH]1)CC3 ; Cc1ccc(Br)c2[nH]c3c(c12)CN(C)CC3 ; NCCCCCCn1c2c(c3cc(F)ccc31)CN(CCc1cnn[nH]1)CC2]

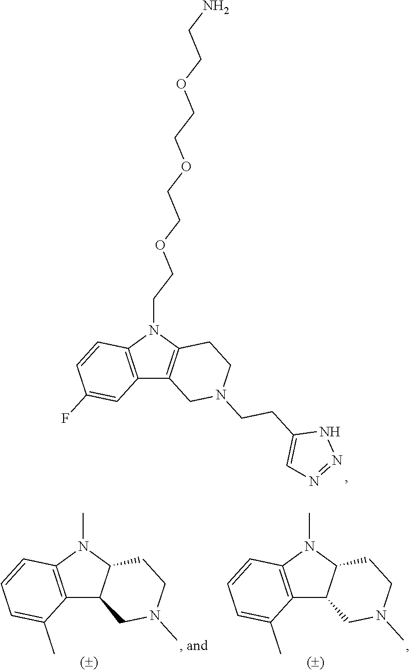

or a pharmaceutically acceptable salt thereof.

In another aspect, provided herein is a method of binding or modulating DDB 1- and CUL4-Associated Factor 1 (DCAF1) in a subject in need thereof, comprising administering a therapeutically effective amount of a compound of Formula I:

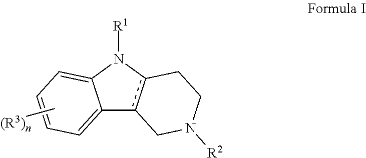

or a pharmaceutically acceptable salt thereof, wherein:
[symbol] is a single bond or a double bond;
R¹ is selected from H, C₁₋₈alkyl, C₂₋₈alkenyl, C₂₋₈alkynyl, C₁₋₈haloalkyl, C₁₋₈heteroalkyl, C₃₋₁₀carbocyclyl, 3- to 10-membered heterocyclyl, C₆₋₁₀aryl, 5- to 10-membered heteroaryl, —C(O)(C₁₋₈alkyl), —C(O)(C₂₋₈alkenyl), —C(O)(C₂₋₈alkynyl), —C(O)(C₁₋₈haloalkyl), —C(O)(C₁₋₈heteroalkyl), —C(O)(C₃₋₁₀carbocyclyl), —C(O)(3- to 10-membered heterocyclyl), —C(O)(C₆₋₁₀aryl), —C(O)(5- to 10-membered heteroaryl), —SO₂(C₁₋₈alkyl), —SO₂(C₂₋₈alkenyl), —SO₂(C₂₋₈alkynyl), —SO₂(C₁₋₈haloalkyl), —SO₂(C₁₋₈heteroalkyl), —SO₂(C₃₋₁₀carbocyclyl), —SO₂(3- to 10-membered heterocyclyl), —SO₂(C₆₋₁₀aryl), and —SO₂(5- to 10-membered heteroaryl), wherein each alkyl, alkenyl, alkynyl, haloalkyl, heteroalkyl, carbocyclyl, heterocyclyl, aryl, and heteroaryl is independently optionally substituted with one or more substituents independently selected from halo, hydroxy, oxo, amino, cyano, nitro, C₁₋₈alkyl, C₂₋₈alkenyl, C₂₋₈alkynyl, C₁₋₈haloalkyl, C₁₋₈heteroalkyl, C₁₋₈alkoxy, C₃₋₁₀carbocyclyl, 3- to 10-membered heterocyclyl, C₆₋₁₀aryl, and 5- to 10-membered heteroaryl;
R² is selected from C₁₋₈alkyl, C₂₋₈alkenyl, C₂₋₈alkynyl, C₁₋₈haloalkyl, C₁₋₈heteroalkyl, C₃₋₁₀carbocyclyl, and 3- to 10-membered heterocyclyl, wherein each alkyl, alkenyl, alkynyl, haloalkyl, heteroalkyl, carbocyclyl, and heterocyclyl is independently optionally substituted with one or more substituents independently selected from halo, hydroxy, oxo, amino, cyano, nitro, C₁₋₈alkyl, C₂₋₈alkenyl, C₂₋₈alkynyl, C₁₋₈haloalkyl, C₁₋₈heteroalkyl, C₁₋₈alkoxy, C₃₋₁₀carbocyclyl, 3- to 10-membered heterocyclyl, C₆₋₁₀aryl, and 5- to 10-membered heteroaryl;
each R³ is independently selected from halo, hydroxy, amino, cyano, nitro, C₁₋₈alkyl, C₂₋₈alkenyl, C₂₋₈alkynyl, C₁₋₈haloalkyl, C₁₋₈heteroalkyl, C₁₋₈alkoxy, C₃₋₁₀carbocyclyl, 3- to 10-membered heterocyclyl, C₆₋₁₀aryl, and 5- to 10-membered heteroaryl; and
n is 0, 1, 2, 3, or 4.

In another aspect, provided herein is a compound of Formula IIa:

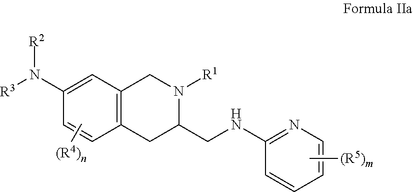

or a pharmaceutically acceptable salt thereof, wherein:
R¹ is selected from H, C₁₋₈alkyl, C₂₋₈alkenyl, C₂₋₈alkynyl, C₁₋₈haloalkyl, and C₁₋₈heteroalkyl;
R² and R³ are independently selected from H, C₁₋₈alkyl, C₂₋₈alkenyl, C₁₋₈alkynyl, C₁₋₈haloalkyl, and C₁₋₈heteroalkyl;
each R⁴ and each R⁵ are independently selected from H, halo, hydroxy, amino, cyano, nitro, C₁₋₈alkyl, C₂₋₈alkenyl, C₂₋₈alkynyl, C₁₋₈haloalkyl, C₁₋₈heteroalkyl, C₁₋₈alkoxy, C₃₋₁₀carbocyclyl, 3- to 10-membered heterocyclyl, C₆₋₁₀aryl, and 5- to 10-membered heteroaryl;
n is 0, 1, 2, or 3; and
m is 0, 1, 2, 3, or 4.

In another aspect, provided herein is a compound selected from:

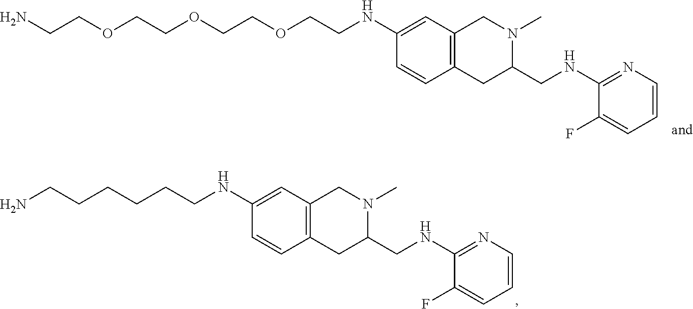 [SMILES: CN1Cc2cc(NCCOCCOCCOCCN)ccc2CC1CNc1ncccc1F ; CN1Cc2cc(NCCCCCCN)ccc2CC1CNc1ncccc1F]

or a pharmaceutically acceptable salt thereof.

In another aspect, provided herein is a method of binding or modulating DDB1- and CUL4-Associated Factor 1 (DCAF1) in a subject in need thereof, comprising administering a therapeutically effective amount of a compound of Formula II:

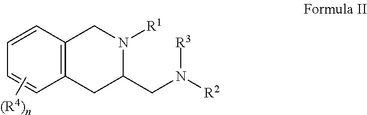

or a pharmaceutically acceptable salt thereof, wherein:
R¹ is selected from H, C₁₋₈alkyl, C₂₋₈alkenyl, C₂₋₈alkynyl, C₁₋₈haloalkyl, and C₁₋₈heteroalkyl;
R² and R³ are independently selected from H, C₁₋₈alkyl, C₂₋₈alkenyl, C₂₋₈alkynyl, C₁₋₈haloalkyl, C₁₋₈heteroalkyl, C₃₋₁₀carbocyclyl, 3- to 10-membered heterocyclyl, C₆₋₁₀aryl, 5- to 10-membered heteroaryl, —C(O)(C₁₋₈alkyl), —C(O)(C₂₋₈alkenyl), —C(O)(C₂₋₈alkynyl), —C(O)(C₁₋₈haloalkyl), —C(O)(C₁₋₈heteroalkyl), —C(O)(C₃₋₁₀carbocyclyl), —C(O)(3- to 10-membered heterocyclyl), —C(O)(C₆₋₁₀aryl), —C(O)(5- to 10-membered heteroaryl), —SO₂(C₁₋₈alkyl), —SO₂(C₂₋₈alkenyl), —SO₂(C₂₋₈alkynyl), —SO₂(C₁₋₈haloalkyl), —SO₂(C₁₋₈heteroalkyl), —SO₂(C₃₋₁₀carbocyclyl), —SO₂(3- to 10-membered heterocyclyl), —SO₂(C₆₋₁₀aryl), and —SO₂(5- to 10-membered heteroaryl), wherein each alkyl, alkenyl, alkynyl, haloalkyl, heteroalkyl, carbocyclyl, heterocyclyl, aryl, and heteroaryl is independently optionally substituted with one or more substituents independently selected from halo, hydroxy, oxo, amino, cyano, nitro, C₁₋₈alkyl, C₂₋₈alkenyl, C₂₋₈alkynyl, C₁₋₈haloalkyl, C₁₋₈heteroalkyl, C₁₋₈alkoxy, C₃₋₁₀carbocyclyl, 3- to 10-membered heterocyclyl, C₆₋₁₀aryl, and 5- to 10-membered heteroaryl;
each R⁴ is independently selected from halo, hydroxy, amino, cyano, nitro, C₁₋₈alkyl, C₂₋₈alkenyl, C₂₋₈alkynyl, C₁₋₈haloalkyl, C₁₋₈heteroalkyl, C₁₋₈alkoxy, C₃₋₁₀carbocyclyl, 3- to 10-membered heterocyclyl, C₆₋₁₀aryl, and 5- to 10-membered heteroaryl; and
n is 0, 1, 2, 3, or 4.

In another aspect, provided herein is a compound selected from:

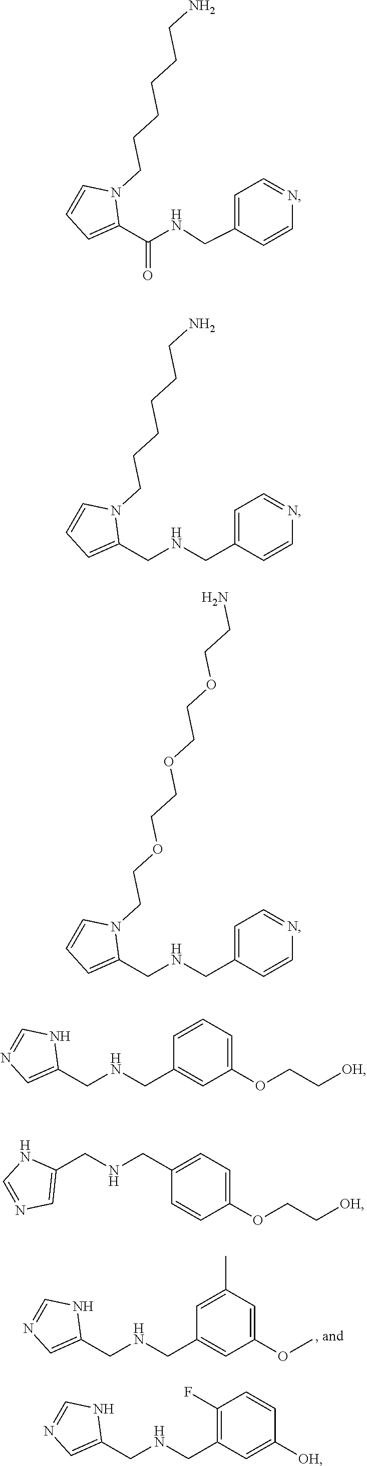

or a pharmaceutically acceptable salt thereof.

In another aspect, provided herein is a method of binding or modulating DDB1- and CUL4-Associated Factor 1 (DCAF1) in a subject in need thereof, comprising administering a therapeutically effective amount of a compound of Formula III:

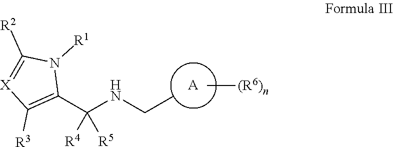

or a pharmaceutically acceptable salt thereof, wherein:
Ring A is selected from null, C₃₋₁₀carbocyclyl, 3- to 10-membered heterocyclyl, C₆₋₁₀aryl, and 5- to 10-membered heteroaryl;
X is selected from N and CR⁷;
R¹ is selected from H, C₁₋₈alkenyl, C₂₋₈alkenyl, C₂₋₈alkynyl, C₁₋₈heteroalkyl, C₃₋₁₀carbocyclyl, 3- to 10-membered heterocyclyl, C₆₋₁₀aryl, and 5- to 10-membered heteroaryl;
R², R³, and R⁷ are independently selected from H, halo, hydroxy, amino, cyano, nitro, C₁₋₈alkyl, C₂₋₈alkenyl, C₂₋₈alkynyl, C₁₋₈haloalkyl, C₁₋₈heteroalkyl, C₁₋₈alkoxy, C₃₋₁₀carbocyclyl, 3- to 10-membered heterocyclyl, C₆₋₁₀aryl, and 5- to 10-membered heteroaryl;
R⁴ and R⁵ are independently selected from H, halo, cyano, nitro, C₁₋₈alkyl, C₂₋₈alkenyl, C₂₋₈alkynyl, C₁₋₈haloalkyl, C₁₋₈heteroalkyl, C₃₋₁₀carbocyclyl, 3- to 10-membered heterocyclyl, C₆₋₁₀aryl, and 5- to 10-membered heteroaryl; or
R⁴ and R⁵ are taken together to form oxo;
each R⁶ is independently selected from H, halo, hydroxy, amino, cyano, nitro, C₁₋₈alkyl, C₂₋₈alkenyl, C₂₋₈alkynyl, C₁₋₈haloalkyl, C₁₋₈heteroalkyl, C₁₋₈alkoxy, C₃₋₁₀carbocyclyl, 3-to 10-membered heterocyclyl, C₆₋₁₀aryl, and 5- to 10-membered heteroaryl; and
n is 0, 1, 2, 3, 4, 5, or 6.

In another aspect, provided herein is a method comprising contacting a compound provided herein with a DDB1- and CUL4-associated factor 1 (DCAF1) protein.

In some embodiments, contacting the compound with the DCAF1 protein comprises administering the compound to a cell.

In some embodiments, contacting the compound with the DCAF1 protein comprises administering the compound to a subject.

In some embodiments, contacting the compound with the DCAF1 protein comprises contacting the compound with a binding region on the DCAF1 protein, the binding region comprising a WD40 domain. In some embodiments, the binding region on the DCAF1 protein comprises one or more of the following DCAF1 residues: THR1097, ALA1137, THR1139, HIS1140, THR1155, HIS1180, TYR1181, ARG1225, CYS1227, ILE1262, VAL1265, ARG1298, VAL1299, VAL1300, LYS1327, PRO1329 or PHE1355.

In some embodiments, the compound binds the DCAF1 protein non-covalently. In some embodiments, the compound binds the DCAF1 protein with a K_{d}≤40 μM. In some embodiments, the compound binds the DCAF1 protein with a K_{d}>40 and ≤70 μM. In some embodiments, the compound binds the DCAF1 protein with a K_{d}>70 and ≤100 μM. In some embodiments, the compound binds the DCAF1 protein with a K_{d}>100 μM.

In another aspect, provided herein is an in vivo modified protein comprising a DDB1- and CUL4-associated factor 1 (DCAF1) protein directly bound to a ligand at a binding region on the DCAF1 protein, the binding region comprising a WD40 domain.

In some embodiments, the binding region on the DCAF1 protein comprises one or more of the following DCAF1 residues: THR1097, ALA1137, THR1139, HIS1140, THR1155, HIS1180, TYR1181, ARG1225, CYS1227, ILE1262, VAL1265, ARG1298, VAL1299, VAL1300, LYS1327, PRO1329 or PHE1355.

In some embodiments, the ligand binds the DCAF1 protein non-covalently. In some embodiments, the ligand binds the DCAF1 protein with a K_{d}≤40 μM. In some embodiments, the ligand binds the DCAF1 protein with a K_{d}>40 and ≤70 μM. In some embodiments, the ligand binds the DCAF1 protein with a K_{d}>70 and ≤100 μM. In some embodiments, the ligand binds the DCAF1 protein with a K_{d}>100 μM.

In some embodiments, the ligand is synthetic.

In some embodiments, the ligand is a small molecule.

In some embodiments, the ligand comprises a compound provided herein.

### INCORPORATION BY REFERENCE

All publications, patents, and patent applications mentioned in this specification are herein incorporated by reference for the specific purposes identified herein.

### BRIEF DESCRIPTION OF THE DRAWINGS

FIG. 1 shows an example of a compound bound to a binding region of a DCAF1 protein.

FIG. 2A-2F show binding data for some compounds described herein.

### DETAILED DESCRIPTION

The ubiquitin pathway plays an important role in the regulation of most cellular processes via an enzymatic cascade, where E1 and E2 enzymes catalyze the activation and conjugation of ubiquitin, and E3s confer reaction specificity through substrate recruitment (Hershko and Ciechanover, 1998; Pickart, 2004). Cullin RING E3 ligases (CRLs) are the largest family of E3 ubiquitin ligases. In the CRL ligase complexes, cullin serves as a scaffold to bind small RING finger protein ROC1 or ROC2 (RBX1 or RBX2) through a C-terminal domain and a linker-substrate receptor dimer or a substrate receptor directly through an N-terminal domain. Mammalian cells express nine distinct cullins, including two cullin 4 (CUL4) proteins: CUL4A and CUL4B, which use DNA damage-binding protein 1 (DDB1) as the linker. DDB1 bridges the interaction between CUL4 and a subset of DDB1 binding WD40 repeat proteins (DWD or DCAFs for DDB1 cullin associated factors). These DCAF proteins function as substrate receptors to target specific substrates to the CRL4 E3 complexes (Jackson and Xiong, 2009). One of the most abundant DCAF proteins is DCAF1 (also known as VprBP).

DCAF1 is evolutionarily conserved in mammals, Drosophila, Xenopus, C. elegans, and Arabidopsis, but has no apparent homolog in yeast (Nakagawa et al., 2013; Schabla et al., 2019). It is ubiquitously expressed in all tissues and organs that have been examined (Zhang et al., 2001). Genetic analyses revealed an essential function of DCAF1 during embryonic development in plants, flies, and mammals, resulting in developmental arrest at the globular stage of Arabidopsis (Zhang et al., 2008), late pupal stage in Drosophila (Tamori et al., 2010) and early embryonic lethality in mice (McCall et al., 2008), respectively.

DCAF1 was first identified as the HIV-1 accessory viral protein R (Vpr) binding protein (Zhang et al., 2001; Zhao et al., 1994), and was subsequently shown to associate with a DDB1-CUL4-ROC1 E3 ubiquitin ligase (CRL4) (Angers et al., 2006; He et al., 2006; Jin et al., 2006). DCAF1 contains multiple functional domains, including a putative protein kinase-like domain (Kim et al., 2013), a chromo domain functions as a mono-methylated substrate recognition pocket (Lee et al., 2012), a putative LisH motif required for dimerization and interacting with H3 Tail (Ahn et al., 2011; Kim et al., 2012), a promiscuous α-helical motif H-box required for binding to DDB1(Fischer et al., 2011; Li et al., 2010), a WD40 repeat region required for binding to DDB1, and an acidic-domain providing interactions with additional protein (Huang and Chen, 2008; Wang et al., 2016). DCAF1 ligands have the potential to be used as anti-viral agents.

Provided herein are compounds, pharmaceutical compositions, and methods for binding or modulating a DDB1- and CUL4-associated factor 1 (DCAF1) protein. Further provided herein are ligand-DCAF1 complexes or in vivo modified DCAF1 proteins.

The DCAF1 protein may be a mammalian DCAF1 protein. The DCAF1 protein may be a human DCAF1 protein. The DCAF1 protein may be encoded by a DCAF1 gene such as NCBI Gene ID: 9730 (updated on Jan. 29, 2021). The DCAF1 protein may include an amino acid sequence. An example of a DCAF1 amino acid sequence is included at UniProt ref. Q9Y4B6 (sequence last modified May 15, 2007). In some embodiments, the DCAF1 protein contains 1507 amino acids, or has a mass of 169 kDa.

### Modified Proteins and Ligand-Protein Complexes

Disclosed herein, in some embodiments, are modified proteins. In some embodiments, the modified protein comprises an in vivo modified protein. In some embodiments, the modified protein comprises an in vitro modified protein. In some embodiments, the modified protein comprises a DDB1- and CUL4-associated factor 1 (DCAF1) protein. In some embodiments, the modified protein comprises an in vivo modified DCAF1 protein. In some embodiments, the DCAF1 protein is bound to a ligand. The ligand may be a compound described herein, for example a compound of Table 1 or formula Ia, I, IIa, II, or III. In some embodiments, the DCAF1 protein is bound to a compound described herein. In some embodiments, the DCAF1 protein is directly bound to the compound. In some embodiments, the binding between the DCAF1 protein and the compound is non-covalent. In some embodiments, the binding between the DCAF1 protein and the compound is covalent. The modified protein may be used in a method described herein. In some embodiments, the ligand is bound to a DCAF1 fragment. In some embodiments, the ligand is bound to a full-length DCAF1 protein.

Disclosed herein, in some embodiments, are ligand-protein complexes. In some embodiments, the ligand-protein complex comprises a DCAF1 protein. In some embodiments of the ligand-protein complex, the DCAF1 protein is bound to a ligand. The ligand may be a compound described herein, for example a compound of Table 1 or formula Ia, I, IIa, II, or III. In some embodiments, the DCAF1 protein is directly bound to the compound. In some embodiments, the binding between the DCAF1 protein and the compound is non-covalent. In some embodiments, the binding between the DCAF1 protein and the compound is covalent. The ligand-protein complex may be formed in vivo. The ligand-protein complex may be formed in vitro. The ligand-protein complex may be used in a method described herein. In some embodiments, the ligand is bound to a DCAF1 fragment. In some embodiments, the ligand is bound to a full-length DCAF1 protein.

Disclosed herein, in some embodiments, are modified proteins or ligand-protein complexes that include a compound described herein bound to a DCAF1 protein. In some embodiments, the DCAF1 protein comprises a binding region. In some embodiments, the compound is bound to the binding region of the DCAF1 protein. In some embodiments, the binding region comprises a WD40 domain. In some embodiments, a DCAF1 fragment comprises a WD40 domain.

In some embodiments, the binding region of the DCAF1 protein comprises an alanine. In some embodiments, the binding region of the DCAF1 protein comprises an arginine. In some embodiments, the binding region of the DCAF1 protein comprises a cysteine. In some embodiments, the binding region of the DCAF1 protein comprises a histidine. In some embodiments, the binding region of the DCAF1 protein comprises a lysine. In some embodiments, the binding region of the DCAF1 protein comprises a proline. In some embodiments, the binding region of the DCAF1 protein comprises a threonine. In some embodiments, the binding region of the DCAF1 protein comprises a tyrosine. In some embodiments, the binding region of the DCAF1 protein comprises a valine.

In some embodiments, the binding region of the DCAF1 protein comprises one or more amino acids after amino acid position 1000, 1050, 1100, 1150, 1200, 1250, 1300, 1350, 1400, 1450, or 1500 of the DCAF1 protein. In some embodiments, the binding region of the DCAF1 protein comprises one or more amino acids before amino acid position 1000, 1050, 1100, 1150, 1200, 1250, 1300, 1350, 1400, 1450, or 1500 of the DCAF1 protein. In some embodiments, the binding region of the DCAF1 protein comprises one or more amino acids between amino acid positions 1095 and 1355 of the DCAF1 protein.

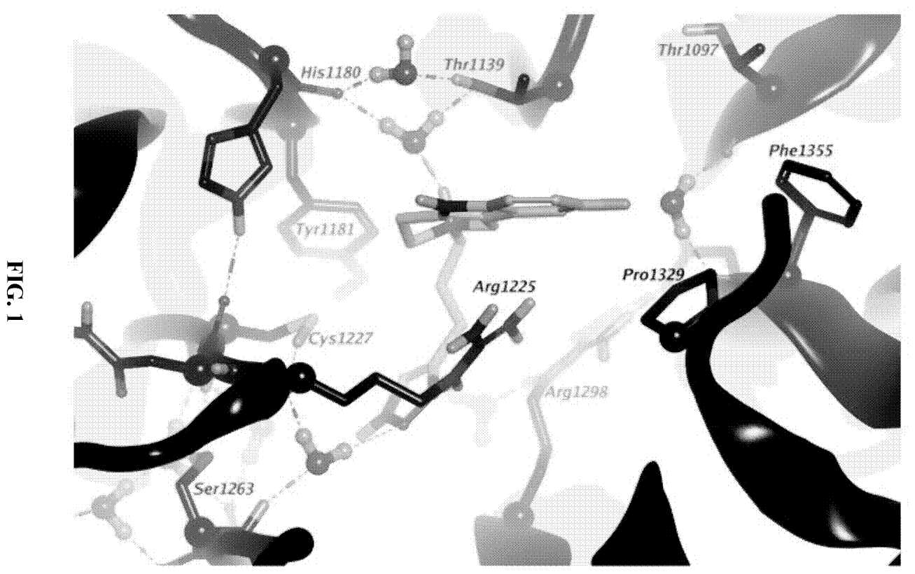

In some embodiments, the binding region of the DCAF1 protein comprises one or more of the following DCAF1 residues: THR1097, ALA1137, THR1139, HIS1140, THR1155, HIS1180, TYR1181, ARG1225, CYS1227, ILE1262, VAL1265, ARG1298, VAL1299, VAL1300, LYS1327, PRO1329 or PHE1355. The binding region may include THR1097, ALA1137, THR1139, HIS1140, THR1155, HIS1180, TYR1181, ARG1225, CYS1227, ILE1262, VAL1265, ARG1298, VAL1299, VAL1300, LYS1327, PRO1329, or PHE1355. In some embodiments, the binding region of the DCAF1 protein comprises THR1097. In some embodiments, the binding region of the DCAF1 protein comprises ALA1137. In some embodiments, the binding region of the DCAF1 protein comprises THR1139. In some embodiments, the binding region of the DCAF1 protein comprises HIS1140. In some embodiments, the binding region of the DCAF1 protein comprises THR1155. In some embodiments, the binding region of the DCAF1 protein comprises HIS1180. In some embodiments, the binding region of the DCAF1 protein comprises TYR1181. In some embodiments, the binding region of the DCAF1 protein comprises ARG1225. In some embodiments, the binding region of the DCAF1 protein comprises CYS1227. In some embodiments, the binding region of the DCAF1 protein comprises ILE1262. In some embodiments, the binding region of the DCAF1 protein comprises VAL1265. In some embodiments, the binding region of the DCAF1 protein comprises ARG1298. In some embodiments, the binding region of the DCAF1 protein comprises VAL1299. In some embodiments, the binding region of the DCAF1 protein comprises VAL1300. In some embodiments, the binding region of the DCAF1 protein comprises LYS1327. In some embodiments, the binding region of the DCAF1 protein comprises PRO1329. In some embodiments, the binding region of the DCAF1 protein comprises PHE1355. In some embodiments, the one or more DCAF1 residues are non-covalently bound to the compound. FIG. 1 shows a docking model of an exemplary compound (Compound A8) that may bind to said binding region of the DCAF1 protein.

In some embodiments, the binding between the DCAF1 protein and the compound comprises one or more of a salt-bridge, a hydrogen bond, a stereoelectronic interaction, and a dispersion contact. In some embodiments, the binding between the DCAF1 protein and the compound comprises a salt-bridge. In some embodiments, the binding between the DCAF1 protein and the compound comprises one or more hydrogen bonds. In some embodiments, the binding between the DCAF1 protein and the compound comprises a stereoelectronic interaction. In some embodiments, the binding between the DCAF1 protein and the compound comprises a dispersion contact.

In some embodiments, the binding between the DCAF1 protein and the ligand comprises a binding affinity with an equilibrium dissociation constant (K_{d}) below 1500 μM, a K_{d} below 1250 μM, a K_{d} below 1000 μM, a K_{d} below 750 μM, a K_{d} below 500 μM, a K_{d} below 450 μM, a K_{d} below 400 μM, a K_{d} below 350 μM, a K_{d} below 300 μM, a K_{d} below 250 μM, a K_{d} below 200 μM, a K_{d} below 150 μM, a K_{d} below 100 μM, a K_{d} below 90 μM, a K_{d} below 80 μM, a K_{d} below 70 μM, a K_{d} below 60 μM, below 50 μM, a K_{d} below 45 μM, a K_{d} below 40 μM, a K_{d} below 35 μM, a K_{d} below 30 μM, a K_{d} below 25 μM, or a K_{d} below 20 μM. In some embodiments, the K_{d} is 100 μM or less. In some embodiments, the K_{d} is 70 μM or less. In some embodiments, the K_{d} is 40 μM or less. In some embodiments, the K_{d} is about 100 μM or less. In some embodiments, the K_{d} is about 70 μM or less. In some embodiments, the K_{d} is about 40 μM or less.

In some embodiments, the binding between the DCAF1 protein and the ligand comprises a binding affinity with a K_{d} above 1250 μM, a K_{d} above 1000 μM, a K_{d} above 750 μM, a K_{d} above 500 μM, a K_{d} above 450 μM, a K_{d} above 400 μM, a K_{d} above 350 μM, a K_{d} above 300 μM, a K_{d} above 250 μM, a K_{d} above 200 μM, a K_{d} above 150 μM, a K_{d} above 100 μM, a K_{d} above 90 μM, a K_{d} above 80 μM, a K_{d} above 70 μM, a K_{d} above 60 μM, above 50 μM, a K_{d} above 45 μM, a K_{d} above 40 μM, a K_{d} above 35 μM, a K_{d} above 30 μM, a K_{d} above 25 μM, a K_{d} above 20 μM, or a K_{d} above 15 μM. In some embodiments, the K_{d} is greater than 100. In some embodiments, the K_{d} is greater than 70. In some embodiments, the K_{d} is greater than 40. In some embodiments, the K_{d} is greater than about 100. In some embodiments, the K_{d} is greater than about 70. In some embodiments, the K_{d} is greater than about 40.

In some embodiments, the binding between the DCAF1 protein and the compound comprises a binding affinity with a K_{d}≤40 uM, a K_{d}>40 and ≤70 uM, a K_{d}>70 and ≤100 uM, or a K_{d}>100 uM. In some embodiments, the binding between the DCAF1 protein and the compound comprises a binding affinity with a K_{d}≤40 uM. In some embodiments, the binding between the DCAF1 protein and the compound comprises a binding affinity with a K_{d} >40 and ≤70 uM. In some embodiments, the binding between the DCAF1 protein and the compound comprises a binding affinity with a K_{d}>70 and ≤100 uM. In some embodiments, the binding between the DCAF1 protein and the compound comprises a binding affinity with a K_{d}>100 uM.

### Compounds

In one aspect, provided herein is a compound of Formula Ia:

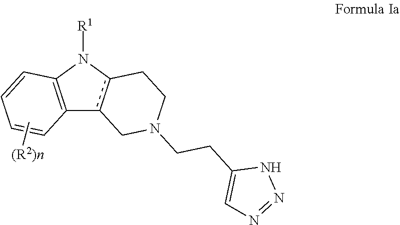

or a pharmaceutically acceptable salt thereof, wherein:
[symbol] is a single bond or a double bond;
R¹ selected from H, C₁₋₁₀alkyl, C₂₋₁₀alkenyl, C₂₋₁₀alkynyl, C₁₋₁₀haloalkyl, and C₁₋₁₀heteroalkyl; each R² is independently selected from H, halo, hydroxy, amino, cyano, nitro, C₁₋₈alkyl, C₂₋₈alkenyl, C₂₋₈alkynyl, C₁₋₈haloalkyl, C₁₋₈heteroalkyl, C₁₋₈alkoxy, C₃₋₁₀carbocyclyl, 3- to 10-membered heterocyclyl, C₆₋₁₀aryl, and 5- to 10-membered heteroaryl; and
n is 0, 1, 2, 3, or 4;
provided that the compound of Formula Ia is not

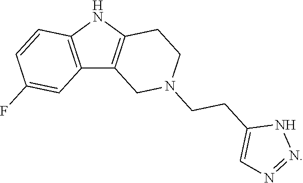 [SMILES: Fc1ccc2[nH]c3c(c2c1)CN(CCc1cnn[nH]1)CC3]

In some embodiments, R¹ is selected from H, C₁₋₁₀alkyl, C₂₋₁₀alkenyl, C₂₋₁₀alkynyl, C₁₋₁₀haloalkyl, and C₁₋₁₀heteroalkyl. In some embodiments, R¹ is selected from C₁₋₁₀alkyl, C₂₋₁₀alkenyl, C₂₋₁₀alkynyl, C₁₋₁₀haloalkyl, and C₁₋₁₀heteroalkyl. In some embodiments, R¹ is selected from C₁₋₁₀haloalkyl and C₁₋₁₀heteroalkyl. In some embodiments, R¹ is H. In some embodiments, R¹ is C₁₋₁₀alkyl. In some embodiments, R¹ is C₂₋₁₀alkenyl. In some embodiments, R¹ is C₂₋₁₀alkynyl. In some embodiments, R¹ is C₁₋₁₀haloalkyl. In some embodiments, R¹ is C₁₋₁₀heteroalkyl.

In some embodiments, each R² is independently selected from H, halo, hydroxy, amino, cyano, nitro, C₁₋₈alkyl, C₂₋₈alkenyl, C₂₋₈alkynyl, C₁₋₈haloalkyl, C₁₋₈heteroalkyl, C₁₋₈alkoxy, C₃₋₁₀carbocyclyl, 3- to 10-membered heterocyclyl, C₆₋₁₀aryl, and 5- to 10-membered heteroaryl. In some embodiments, each R² is independently selected from H, halo, hydroxy, amino, cyano, nitro, C₁₋₈alkyl, C₂₋₈alkenyl, C₂₋₈alkynyl, C₁₋₈haloalkyl, C₁₋₈heteroalkyl, and C₁₋₈alkoxy. In some embodiments, each R² is independently selected from H, halo, hydroxy, amino, cyano, nitro, C₁₋₈haloalkyl, C₁₋₈heteroalkyl, and C₁₋₈alkoxy. In some embodiments, each R² is independently selected from H, halo, hydroxy, amino, cyano, and nitro. In some embodiments, each R² is independently H. In some embodiments, each R² is independently halo. In some embodiments, each R² is independently hydroxy. In some embodiments, each R² is independently amino. In some embodiments, each R² is independently cyano. In some embodiments, each R² is independently nitro. In some embodiments, each R² is independently C₁₋₈alkyl. In some embodiments, each R² is independently C₂₋₈alkenyl. In some embodiments, each R² is independently C₂₋₈alkynyl. In some embodiments, each R² is independently C₁₋₈haloalkyl. In some embodiments, each R² is independently C₁₋₈heteroalkyl. In some embodiments, each R² is independently C₁₋₈alkoxy. In some embodiments, each R² is independently C₃₋₁₀carbocyclyl. In some embodiments, each R² is independently 3- to 10-membered heterocyclyl. In some embodiments, each R² is independently C₆₋₁₀aryl. In some embodiments, each R² is independently 5- to 10-membered heteroaryl.

In some embodiments, n is 0, 1, 2, 3, or 4. In some embodiments, n is 0. In some embodiments, n is 1. In some embodiments, n is 2. In some embodiments, n is 3. In some embodiments, n is 4.

In another aspect, provided herein is a compound selected from:

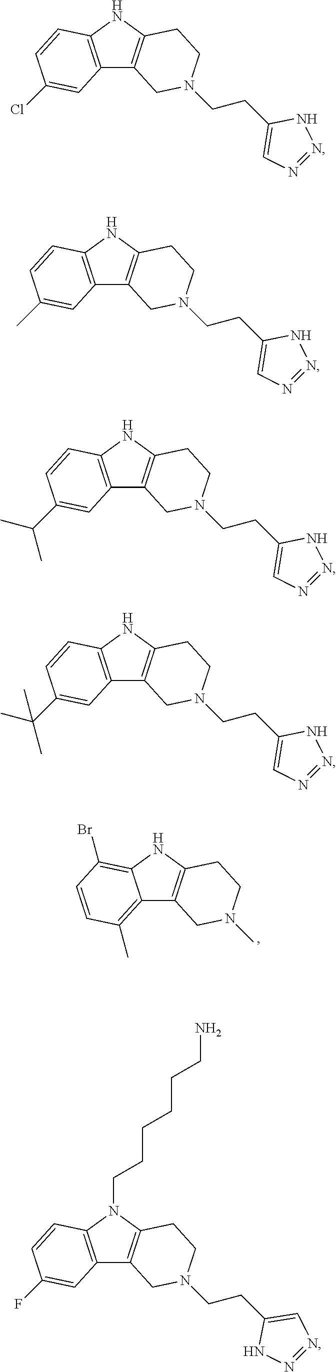

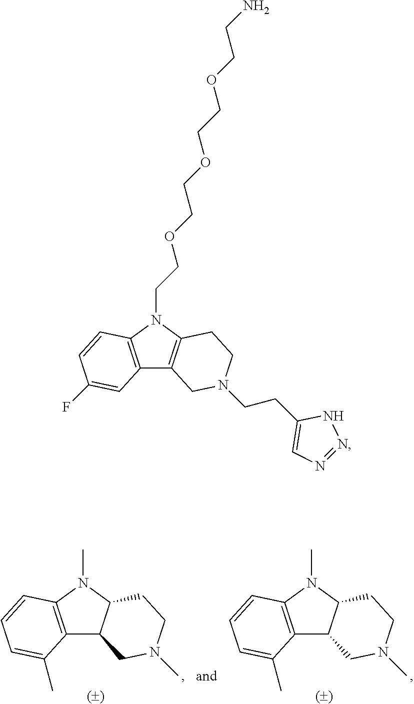 [SMILES: NCCOCCOCCOCCn1c2c(c3cc(F)ccc31)CN(CCc1cnn[nH]1)CC2 ; Cc1cccc2c1[C@@H]1CN(C)CC[C@H]1N2C ; "," ; "and" ; Cc1cccc2c1[C@H]1CN(C)CC[C@H]1N2C ; ","]

or a pharmaceutically acceptable salt thereof.

In another aspect, provided herein is a method of binding or modulating DDB1- and CUL4-Associated Factor 1 (DCAF1) in a subject in need thereof, comprising administering a therapeutically effective amount of a compound of Formula I:

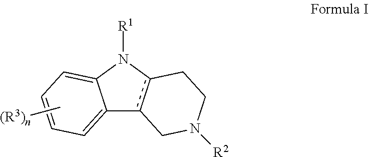

or a pharmaceutically acceptable salt thereof, wherein:
[symbol] is a single bond or a double bond;
R¹ is selected from H, C₁₋₈alkyl, C₂₋₈alkenyl, C₂₋₈alkynyl, C₁₋₈haloalkyl, C₁₋₈heteroalkyl, C₃₋₁₀carbocyclyl, 3- to 10-membered heterocyclyl, C6- 1 oaryl, 5- to 10-membered heteroaryl, —C(O)(C₁₋₈alkyl), —C(O)(C₂₋₈alkenyl), —C(O)(C₂₋₈alkynyl), —C(O)(C₁₋₈haloalkyl), —C(O)(C₁₋₈heteroalkyl), —C(O)(C₃₋₁₀carbocyclyl), —C(O)(3- to 10-membered heterocyclyl), —C(O)(C₆₋₁₀aryl), —C(O)(5- to 10-membered heteroaryl), —SO₂(C₁₋₈alkyl), —SO₂(C₂₋₈alkenyl), —SO₂(C₂₋₈alkynyl), —SO₂(C₁₋₈haloalkyl), —SO₂(C₁₋₈heteroalkyl), —SO₂(C₃₋₁₀carbocyclyl), —SO₂(3- to 10-membered heterocyclyl), —SO₂(C₆₋₁₀aryl), and —SO₂(5- to 10-membered heteroaryl), wherein each alkyl, alkenyl, alkynyl, haloalkyl, heteroalkyl, carbocyclyl, heterocyclyl, aryl, and heteroaryl is independently optionally substituted with one or more substituents independently selected from halo, hydroxy, oxo, amino, cyano, nitro, C₁₋₈alkyl, C₂₋₈alkenyl, C₂₋₈alkynyl, C₁₋₈haloalkyl, C₁₋₈heteroalkyl, C₁₋₈alkoxy, C₃₋₁₀carbocyclyl, 3- to 10-membered heterocyclyl, C₆₋₁₀aryl, and 5- to 10-membered heteroaryl;
R² is selected from C₁₋₈alkyl, C₂₋₈alkenyl, C₂₋₈alkynyl, C₁₋₈haloalkyl, C₁₋₈heteroalkyl, C₃₋₁₀carbocyclyl, and 3- to 10-membered heterocyclyl, wherein each alkyl, alkenyl, alkynyl, haloalkyl, heteroalkyl, carbocyclyl, and heterocyclyl, is independently optionally substituted with one or more substituents independently selected from halo, hydroxy, oxo, amino, cyano, nitro, C₁₋₈alkyl, C₂₋₈alkenyl, C₂₋₈alkynyl, C₁₋₈haloalkyl, C₁₋₈heteroalkyl, C₁₋₈alkoxy, C₃₋₁₀carbocyclyl, 3- to 10-membered heterocyclyl, C₆₋₁₀aryl, and 5- to 10-membered heteroaryl;
each R³ is independently selected from halo, hydroxy, amino, cyano, nitro, C₁₋₈alkyl, C₂₋₈alkenyl, C₂₋₈alkynyl, C₁₋₈haloalkyl, C₁₋₈heteroalkyl, C₁₋₈alkoxy, C₃₋₁₀carbocyclyl, 3- to 10-membered heterocyclyl, C₆₋₁₀aryl, and 5- to 10-membered heteroaryl; and
n is 0, 1, 2, 3, or 4.

In some embodiments, R¹ is selected from H, C₁₋₈alkyl, C₂₋₈alkenyl, C₂₋₈alkynyl, C₁₋₈haloalkyl, C₁₋₈heteroalkyl, C₃₋₁₀carbocyclyl, 3- to 10-membered heterocyclyl, C₆₋₁₀aryl, 5- to 10-membered heteroaryl, —C(O)(C₁₋₈alkyl), —C(O)(C₂₋₈alkenyl), —C(O)(C₂₋₈alkynyl), —C(O)(C₁₋₈haloalkyl), —C(O)(C₁₋₈heteroalkyl), —C(O)(C₃₋₁₀carbocyclyl), —C(O)(3- to 10-membered heterocyclyl), —C(O)(C₆₋₁₀aryl), —C(O) (5- to 10-membered heteroaryl), —SO₂(C₁₋₈alkyl), —SO₂(C₂₋₈alkenyl), —SO₂(C₂₋₈alkynyl), —SO₂(C₁₋₈haloalkyl), —SO₂(C₁₋₈heteroalkyl), —SO₂(C₃₋₁₀carbocyclyl), —SO₂(3- to 10-membered heterocyclyl), —SO₂(C₆₋₁₀aryl), and —SO₂(5- to 10-membered heteroaryl), wherein each alkyl, alkenyl, alkynyl, haloalkyl, heteroalkyl, carbocyclyl, heterocyclyl, aryl, and heteroaryl is independently optionally substituted with one or more substituents independently selected from halo, hydroxy, oxo, amino, cyano, nitro, C₁₋₈alkyl, C₂₋₈alkenyl, C₂₋₈alkynyl, C₁₋₈haloalkyl, C₁₋₈heteroalkyl, C₁₋₈alkoxy, C₃₋₁₀carbocyclyl, 3- to 10-membered heterocyclyl, C₆₋₁₀aryl, and 5- to 10-membered heteroaryl. In some embodiments, R¹ is selected from H, C₁₋₈alkyl, C₂₋₈alkenyl, C₂₋₈alkynyl, C₁₋₈haloalkyl, C₁₋₈heteroalkyl, —C(O)(C₁₋₈alkyl), —C(O)(C₂₋₈alkenyl), —C(O)(C₂₋₈alkynyl), —C(O)(C₁₋₈haloalkyl), —C(O)(C₁₋₈heteroalkyl), —SO₂(C₁₋₈alkyl), —SO₂(C₂₋₈alkenyl), —SO₂(C₂₋₈alkynyl), —SO₂(C₁₋₈haloalkyl), and —SO₂(C₁₋₈heteroalkyl), wherein each alkyl, alkenyl, alkynyl, haloalkyl, and heteroalkyl is independently optionally substituted with one or more substituents independently selected from halo, hydroxy, oxo, amino, cyano, nitro, C₁₋₈alkyl, C₂₋₈alkenyl, C₂₋₈alkynyl, C₁₋₈haloalkyl, C₁₋₈heteroalkyl, and C₁₋₈alkoxy. In some embodiments, R¹ is selected from H, C₁₋₈alkyl, C₁₋₈haloalkyl, C₁₋₈heteroalkyl, —C(O)(C₁₋₈alkyl), —C(O)(C₁₋₈haloalkyl), —C(O)(C₁₋₈heteroalkyl), —SO₂(C₁₋₈alkyl), —SO₂(C₁₋₈haloalkyl), and —SO₂(C₁₋₈heteroalkyl), wherein each alkyl, haloalkyl, and heteroalkyl is independently optionally substituted with one or more substituents independently selected from halo, hydroxy, oxo, amino, cyano, nitro, C₁₋₈alkyl, C₁₋₈haloalkyl, C₁₋₈heteroalkyl, and C₁₋₈alkoxy. In some embodiments, R¹ is selected from H, C₁₋₈alkyl, C₁₋₈haloalkyl, and C₁₋₈heteroalkyl, wherein each alkyl, haloalkyl, and heteroalkyl is independently optionally substituted with one or more substituents independently selected from halo, hydroxy, oxo, amino, cyano, and nitro.

In some embodiments, R² is selected from C₁₋₈alkyl, C₂₋₈alkenyl, C₂₋₈alkynyl, C₁₋₈haloalkyl, and C₁₋₈heteroalkyl, C₃₋₁₀carbocyclyl, and 3- to 10-membered heterocyclyl, wherein each alkyl, alkenyl, alkynyl, haloalkyl, heteroalkyl, carbocyclyl, and heterocyclyl, is independently optionally substituted with one or more substituents independently selected from halo, hydroxy, oxo, amino, cyano, nitro, C₁₋₈alkyl, C₂₋₈alkenyl, C₂₋₈alkynyl, C₁₋₈haloalkyl, C₁₋₈heteroalkyl, C₁₋₈alkoxy, C₃₋₁₀carbocyclyl, 3-to 10-membered heterocyclyl, C₆₋₁₀aryl, and 5- to 10-membered heteroaryl. In some embodiments, R² is selected from C₁₋₈alkyl, C₁₋₈haloalkyl, C₁₋₈heteroalkyl, C₃₋₁₀carbocyclyl, and 3- to 10-membered heterocyclyl, wherein each alkyl, haloalkyl, heteroalkyl, carbocyclyl, and heterocyclyl, is independently optionally substituted with one or more substituents independently selected from halo, hydroxy, oxo, amino, cyano, nitro, C₁₋₈alkyl, C₁₋₈haloalkyl, C₁₋₈heteroalkyl, and C₁₋₈alkoxy. In some embodiments, R² is selected from C₁₋₈alkyl, C₁₋₈haloalkyl, C₁₋₈heteroalkyl, C₃₋₁₀carbocyclyl, and 3- to 10-membered heterocyclyl, wherein each alkyl, haloalkyl, heteroalkyl, carbocyclyl, and heterocyclyl, is independently optionally substituted with one or more substituents independently selected from halo, hydroxy, oxo, amino, cyano, and nitro.

In some embodiments, each R³ is independently selected from halo, hydroxy, amino, cyano, nitro, C₁₋₈alkyl, C₂₋₈alkenyl, C₂₋₈alkynyl, C₁₋₈haloalkyl, C₁₋₈heteroalkyl, C₁₋₈alkoxy, C₃₋₁₀carbocyclyl, 3- to 10-membered heterocyclyl, C₆₋₁₀aryl, and 5- to 10-membered heteroaryl. In some embodiments, each R³ is independently selected from halo, hydroxy, amino, cyano, nitro, C₁₋₈alkyl, C₁₋₈haloalkyl, C₁₋₈heteroalkyl, C₁₋₈alkoxy, C₃₋₁₀carbocyclyl, 3- to 10-membered heterocyclyl, C₆₋₁₀aryl, and 5- to 10-membered heteroaryl. In some embodiments, each R³ is independently selected from halo, hydroxy, amino, cyano, nitro, C₁₋₈alkyl, C₁₋₈haloalkyl, C₁₋₈heteroalkyl, and C₁₋₈alkoxy. In some embodiments, each R³ is independently selected from halo, hydroxy, amino, cyano, and nitro. In some embodiments, each R³ is independently halo. In some embodiments, each R³ is independently hydroxy. In some embodiments, each R³ is independently amino. In some embodiments, each R³ is independently cyano. In some embodiments, each R³ is independently nitro. In some embodiments, each R³ is independently C₁₋₈alkyl. In some embodiments, each R³ is independently C₂₋₈alkenyl. In some embodiments, each R³ is independently C₂₋₈alkynyl. In some embodiments, each R³ is independently C₁₋₈haloalkyl. In some embodiments, each R³ is independently C₁₋₈heteroalkyl. In some embodiments, each R³ is independently C₁₋₈alkoxy. In some embodiments, each R³ is independently In some embodiments, each R³ is independently C₃₋₁₀carbocyclyl. In some embodiments, each R³ is independently 3- to 10-membered heterocyclyl. In some embodiments, each R³ is independently C₆₋₁₀aryl. In some embodiments, each R³ is independently 5- to 10-membered heteroaryl.

In some embodiments, n is 0, 1, 2, 3, or 4. In some embodiments, n is 0. In some embodiments, n is 1. In some embodiments, n is 2. In some embodiments, n is 3. In some embodiments, n is 4.

In another aspect, provided herein is a compound of Formula IIa:

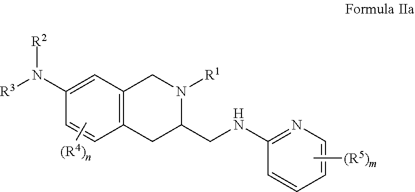

or a pharmaceutically acceptable salt thereof, wherein:
R¹ is selected from H, C₁₋₈alkyl, C₂₋₈alkenyl, C₂₋₈alkynyl, C₁₋₈haloalkyl, and C₁₋₈heteroalkyl;
R² and R³ are independently selected from H, C₁₋₈alkyl, C₂₋₈alkenyl, C₂₋₈alkynyl, C₁₋₈haloalkyl, and C₁₋₈heteroalkyl;
each R⁴ and each R⁵ are independently selected from H, halo, hydroxy, amino, cyano, nitro, C₁₋₈alkyl, C₂₋₈alkenyl, C₂₋₈alkynyl, C₁₋₈haloalkyl, C₁₋₈heteroalkyl, C₁₋₈alkoxy, C₃₋₁₀carbocyclyl, 3- to 10-membered heterocyclyl, C₆₋₁₀aryl, and 5- to 10-membered heteroaryl;
n is 0, 1, 2, or 3; and
m is 0, 1, 2, 3, or 4.

In some embodiments, R¹ is selected from H, C₁₋₈alkyl, C₂₋₈alkenyl, C₂₋₈alkynyl, C₁₋₈haloalkyl, and C₁₋₈heteroalkyl. In some embodiments, R¹ is selected from H, C₁₋₈alkyl, C₁₋₈haloalkyl, and C₁₋₈heteroalkyl. In some embodiments, R¹ is selected from H and C₁₋₈alkyl. In some embodiments, R¹ is H. In some embodiments, R¹ is C₁₋₈alkyl. In some embodiments, R¹ is C₂₋₈alkenyl. In some embodiments, R¹ is C₂₋₈alkynyl. In some embodiments, R¹ is C₁₋₈haloalkyl. In some embodiments, R¹ is C₁₋₈heteroalkyl.

In some embodiments, R² and R³ are independently selected from H, C₁₋₈alkyl, C₂₋₈alkenyl, C₂₋₈alkynyl, C₁₋₈haloalkyl, and C₁₋₈heteroalkyl. In some embodiments, R² and R³ are independently selected from H, C₁₋₈alkyl, C₁₋₈haloalkyl, and C₁₋₈heteroalkyl. In some embodiments, R² and R³ are independently selected from H and C₁₋₈alkyl. In some embodiments, R² is H. In some embodiments, R² is C₁₋₈alkyl. In some embodiments, R² is C₂₋₈alkenyl. In some embodiments, R² is C₂₋₈alkynyl. In some embodiments, R² is C₁₋₈haloalkyl. In some embodiments, R² is C₁₋₈heteroalkyl. In some embodiments, R³ is H. In some embodiments, R³ is C₁₋₈alkyl. In some embodiments, R³ is C₂₋₈alkenyl. In some embodiments, R³ is C₂₋₈alkynyl. In some embodiments, R³ is C₁₋₈haloalkyl. In some embodiments, R³ is C₁₋₈heteroalkyl.

In some embodiments, each R⁴ and each R⁵ are independently selected from H, halo, hydroxy, amino, cyano, nitro, C₁₋₈alkyl, C₂₋₈alkenyl, C₂₋₈alkynyl, C₁₋₈haloalkyl, C₁₋₈heteroalkyl, C₁₋₈alkoxy, C₃₋₁₀carbocyclyl, 3- to 10-membered heterocyclyl, C₆₋₁₀aryl, and 5- to 10-membered heteroaryl. In some embodiments, each R⁴ and each R⁵ are independently selected from H, halo, hydroxy, amino, cyano, nitro, C₁₋₈alkyl, C₁₋₈haloalkyl, C₁₋₈heteroalkyl, C₁₋₈alkoxy, C₃₋₁₀carbocyclyl, 3- to 10-membered heterocyclyl, C₆₋₁₀aryl, and 5- to 10-membered heteroaryl. In some embodiments, each R⁴ and each R⁵ are independently selected from H, halo, hydroxy, amino, cyano, nitro, C₁₋₈alkyl, C₁₋₈haloalkyl, C₁₋₈heteroalkyl, and C₁₋₈alkoxy. In some embodiments, each R⁴ and each R⁵ are independently selected from H, halo, hydroxy, amino, cyano, and nitro. In some embodiments, each R⁴ is independently H. In some embodiments, each R⁴ is independently halo. In some embodiments, each R⁴ is independently is hydroxy. In some embodiments, each R⁴ is independently amino. In some embodiments, each R⁴ is independently cyano. In some embodiments, each R⁴ is independently nitro. In some embodiments, each R⁴ is independently C₁₋₈alkyl. In some embodiments, each R⁴ is independently C₂₋₈alkenyl. In some embodiments, each R⁴ is independently C₂₋₈alkynyl. In some embodiments, each R⁴ is independently C₁₋₈haloalkyl. In some embodiments, each R⁴ is independently C₁₋₈heteroalkyl. In some embodiments, each R⁴ is independently C₁₋₈alkoxy. In some embodiments, each R⁴ is independently C₃₋₁₀carbocyclyl. In some embodiments, each R⁴ is independently 3- to 10-membered heterocyclyl. In some embodiments, each R⁴ is independently C₆₋₁₀aryl. In some embodiments, each R⁴ is independently 5- to 10-membered heteroaryl. In some embodiments, each R⁵ is independently H. In some embodiments, each R⁵ is independently halo. In some embodiments, each R⁵ is independently hydroxy. In some embodiments, each R⁵ is independently amino. In some embodiments, each R⁵ is independently cyano. In some embodiments, each R⁵ is independently nitro. In some embodiments, each R⁵ is independently C₁₋₈alkyl. In some embodiments, each R⁵ is independently C₂₋₈alkenyl. In some embodiments, each R⁵ is independently C₂₋₈alkynyl. In some embodiments, each R⁵ is independently C₁₋₈haloalkyl. In some embodiments, each R⁵ is independently C₁₋₈heteroalkyl. In some embodiments, each R⁵ is independently C₁₋₈alkoxy. In some embodiments, each R⁵ is independently C₃₋₁₀carbocyclyl. In some embodiments, each R⁵ is independently 3- to 10-membered heterocyclyl. In some embodiments, each R⁵ is independently C₆₋₁₀aryl. In some embodiments, each R⁵ is independently 5- to 10-membered heteroaryl.

In some embodiments, n is 0, 1, 2, or 3. In some embodiments, n is 0. In some embodiments, n is 1. In some embodiments, n is 2. In some embodiments, n is 3.

In some embodiments, m is 0, 1, 2, 3, or 4. In some embodiments, m is 0. In some embodiments, m is 1. In some embodiments, m is 2. In some embodiments, m is 3. In some embodiments, m is 4.

In another aspect, provided herein is a compound selected from:

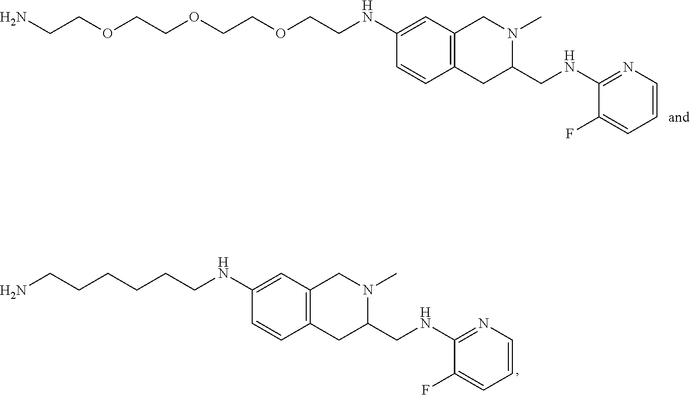 [SMILES: CN1Cc2cc(NCCOCCOCCOCCN)ccc2CC1CNc1ncccc1F ; "and" ; CN1Cc2cc(NCCCCCCN)ccc2CC1CNc1ncccc1F ; ","]

or a pharmaceutically acceptable salt thereof.

In another aspect, provided herein is a method of binding or modulating DDB1- and CUL4-Associated Factor 1 (DCAF1) in a subject in need thereof, comprising administering a therapeutically effective amount of a compound of Formula II:

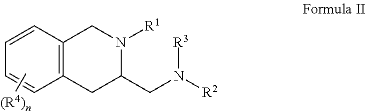

or a pharmaceutically acceptable salt thereof, wherein:
R¹ is selected from H, C₁₋₈alkyl, C₂₋₈alkenyl, C₂₋₈alkynyl, C₁₋₈haloalkyl, and C₁₋₈heteroalkyl;
R² and R³ are independently selected from H, C₁₋₈alkyl, C₂₋₈alkenyl, C₂₋₈alkynyl, C₁₋₈haloalkyl, C₁₋₈heteroalkyl, C₃₋₁₀carbocyclyl, 3- to 10-membered heterocyclyl, C₆₋₁₀aryl, 5- to 10-membered heteroaryl, —C(O)(C₁₋₈alkyl), —C(O)(C₂₋₈alkenyl), —C(O)(C₂₋₈alkynyl), —C(O)(C₁₋₈haloalkyl), —C(O)(C₁₋₈heteroalkyl), —C(O)(C₃₋₁₀carbocyclyl), —C(O)(3- to 10-membered heterocyclyl), —C(O)(C₆₋₁₀aryl), —C(O)(5- to 10-membered heteroaryl), —SO₂(C₁₋₈alkyl), —SO₂(C₂₋₈alkenyl), —SO₂(C₂₋₈alkynyl), —SO₂(C₁₋₈haloalkyl), —SO₂(C₁₋₈heteroalkyl), —SO₂(C₃₋₁₀carbocyclyl), —SO₂(3- to 10-membered heterocyclyl), —SO₂(C₆₋₁₀aryl), and —SO₂(5- to 10-membered heteroaryl), wherein each alkyl, alkenyl, alkynyl, haloalkyl, heteroalkyl, carbocyclyl, heterocyclyl, aryl, and heteroaryl is independently optionally substituted with one or more substituents independently selected from halo, hydroxy, oxo, amino, cyano, nitro, C₁₋₈alkyl, C₂₋₈alkenyl, C₂₋₈alkynyl, C₁₋₈haloalkyl, C₁₋₈heteroalkyl, C₁₋₈alkoxy, C₃₋₁₀carbocyclyl, 3- to 10-membered heterocyclyl, C₆₋₁₀aryl, and 5- to 10-membered heteroaryl;
each R⁴ is independently selected from halo, hydroxy, amino, cyano, nitro, C₁₋₈alkyl, C₂₋₈alkenyl, C₂₋₈alkynyl, C₁₋₈haloalkyl, C₁₋₈heteroalkyl, C₁₋₈alkoxy, C₃₋₁₀carbocyclyl, 3- to 10-membered heterocyclyl, C₆₋₁₀aryl, and 5- to 10-membered heteroaryl; and
n is 0, 1, 2, 3, or 4.

In some embodiments, R¹ is selected from H, C₁₋₈alkyl, C₂₋₈alkenyl, C₂₋₈alkynyl, C₁₋₈haloalkyl, and C₁₋₈heteroalkyl. In some embodiments, R¹ is selected from H, C₁₋₈alkyl, C₁₋₈haloalkyl, and C₁₋₈heteroalkyl. In some embodiments, R¹ is selected from H and C₁₋₈alkyl. In some embodiments, R¹ is H. In some embodiments, R¹ is C₁₋₈alkyl. In some embodiments, R¹ is C₂₋₈alkenyl. In some embodiments, R¹ is C₂₋₈alkynyl. In some embodiments, R¹ is C₁₋₈haloalkyl. In some embodiments, R¹ is C₁₋₈heteroalkyl.

In some embodiments, R² and R³ are independently selected from H, C₁₋₈alkyl, C₂₋₈alkenyl, C₂₋₈alkynyl, C₁₋₈haloalkyl, C₁₋₈heteroalkyl, C₃₋₁₀carbocyclyl, 3- to 10-membered heterocyclyl, C₆₋₁₀aryl, 5- to 10-membered heteroaryl, —C(O)(C₁₋₈alkyl), —C(O)(C₂₋₈alkenyl), —C(O)(C₂₋₈alkynyl), —C(O)(C₁₋₈haloalkyl), —C(O)(C₁₋₈heteroalkyl), —C(O)(C₃₋₁₀carbocyclyl), —C(O)(3- to 10-membered heterocyclyl), —C(O)(C₆₋₁₀aryl), —C(O)(5- to 10-membered heteroaryl), —SO₂(C₁₋₈alkyl), —SO₂(C₂₋₈alkenyl), —SO₂(C₂₋₈alkynyl), —SO₂(C₁₋₈haloalkyl), —SO₂(C₁₋₈heteroalkyl), —SO₂(C₃₋₁₀carbocyclyl), —SO₂(3- to 10-membered heterocyclyl), —SO₂(C₆₋₁₀aryl), and —SO₂(5- to 10-membered heteroaryl), wherein each alkyl, alkenyl, alkynyl, haloalkyl, heteroalkyl, carbocyclyl, heterocyclyl, aryl, and heteroaryl is independently optionally substituted with one or more substituents independently selected from halo, hydroxy, oxo, amino, cyano, nitro, C₁₋₈alkyl, C₂₋₈alkenyl, C₂₋₈alkynyl, C₁₋₈haloalkyl, C₁₋₈heteroalkyl, C₁₋₈alkoxy, C₃₋₁₀carbocyclyl, 3- to 10-membered heterocyclyl, C₆₋₁₀aryl, and 5- to 10-membered heteroaryl. In some embodiments, R² and R³ are independently selected from H, C₁₋₈alkyl, C₁₋₈haloalkyl, C₁₋₈heteroalkyl, C₃₋₁₀carbocyclyl, 3- to 10-membered heterocyclyl, C₆₋₁₀aryl, 5- to 10-membered heteroaryl, —C(O)(C₁₋₈alkyl), —C(O)(C₁₋₈haloalkyl), —C(O)(C₁₋₈heteroalkyl), —C(O)(C₃₋₁₀carbocyclyl), —C(O)(3- to 10-membered heterocyclyl), —C(O)(C₆₋₁₀aryl), —C(O)(5- to 10-membered heteroaryl), —SO₂(C₁₋₈alkyl), —SO₂(C₁₋₈haloalkyl), —SO₂(C₁₋₈heteroalkyl), —SO₂(C₃₋₁₀carbocyclyl), —SO₂(3- to 10-membered heterocyclyl), —SO₂(C₆₋₁₀aryl), and —SO₂(5- to 10-membered heteroaryl), wherein each alkyl, haloalkyl, heteroalkyl, carbocyclyl, heterocyclyl, aryl, and heteroaryl is independently optionally substituted with one or more substituents independently selected from halo, hydroxy, oxo, amino, cyano, nitro, C₁₋₈alkyl, C₁₋₈haloalkyl, C₁₋₈heteroalkyl, C₁₋₈alkoxy, C₃₋₁₀carbocyclyl, 3- to 10-membered heterocyclyl, C₆₋₁₀aryl, and 5- to 10-membered heteroaryl. In some embodiments, R² and R³ are independently selected from H, C₁₋₈alkyl, C₁₋₈haloalkyl, C₁₋₈heteroalkyl, —C(O)(C₁₋₈alkyl), —C(O)(C₁₋₈haloalkyl), —C(O)(C₁₋₈heteroalkyl), —SO₂(C₁₋₈alkyl), —SO₂(C₁₋₈haloalkyl), and —SO₂(C₁₋₈heteroalkyl), wherein each alkyl, haloalkyl, and heteroalkyl is independently optionally substituted with one or more substituents independently selected from halo, hydroxy, oxo, amino, cyano, nitro, C₁₋₈alkyl, C₁₋₈haloalkyl, C₁₋₈heteroalkyl, and C₁₋₈alkoxy. In some embodiments, R² and R³ are independently selected from H, C₁₋₈alkyl, C₁₋₈haloalkyl, and C₁₋₈heteroalkyl, wherein each alkyl, haloalkyl, and heteroalkyl is independently optionally substituted with one or more substituents independently selected from halo, hydroxy, oxo, amino, cyano, and nitro. In some embodiments, R² and R³ are independently selected from H and C₁₋₈alkyl.

In some embodiments, each R⁴ is independently halo. In some embodiments, each R⁴ is independently hydroxy. In some embodiments, each R⁴ is independently amino. In some embodiments, each R⁴ is independently cyano. In some embodiments, each R⁴ is independently nitro. In some embodiments, each R⁴ is independently C₁₋₈alkyl. In some embodiments, each R⁴ is independently C₂₋₈alkenyl. In some embodiments, each R⁴ is independently C₂₋₈alkynyl. In some embodiments, each R⁴ is independently C₁₋₈haloalkyl. In some embodiments, each R⁴ is independently C₁₋₈heteroalkyl. In some embodiments, each R⁴ is independently C₁₋₈alkoxy. In some embodiments, each R⁴ is independently C₃₋₁₀carbocyclyl. In some embodiments, each R⁴ is independently 3- to 10-membered heterocyclyl. In some embodiments, each R⁴ is independently C₆₋₁₀aryl. In some embodiments, each R⁴ is independently 5- to 10-membered heteroaryl.

In some embodiments, n is 0, 1, 2, 3, or 4. In some embodiments, n is 0. In some embodiments, n is 1. In some embodiments, n is 2. In some embodiments, n is 3. In some embodiments, n is 4.

In another aspect, provided herein is a compound selected from:

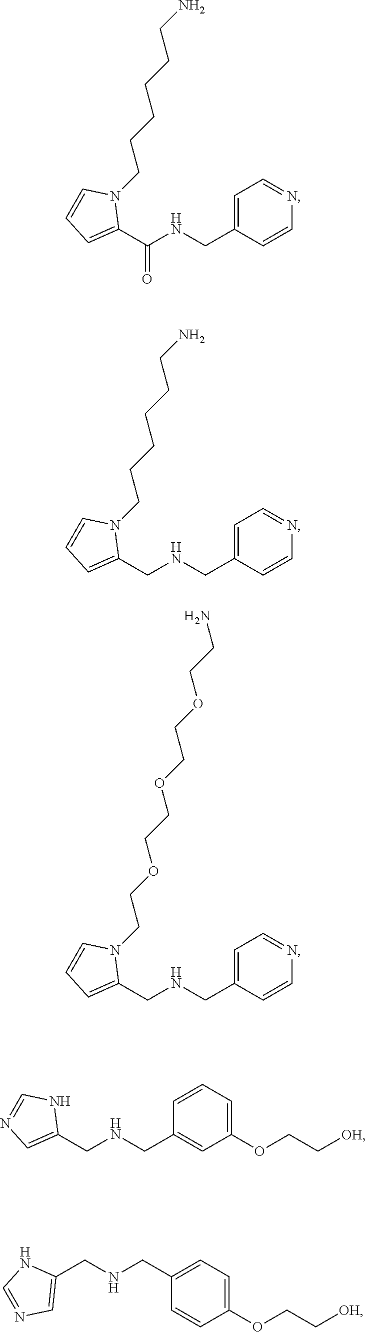

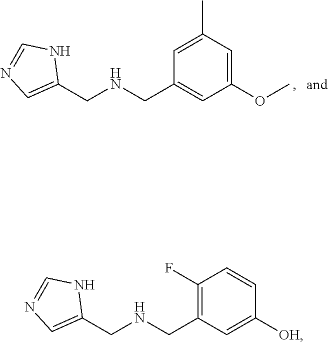 [SMILES: COc1cc(C)cc(CNCc2cnc[nH]2)c1 ; "," ; "and" ; Oc1ccc(F)c(CNCc2cnc[nH]2)c1]

or a pharmaceutically acceptable salt thereof.

In another aspect, provided herein is a method of binding or modulating DDB 1- and CUL4-Associated Factor 1 (DCAF1) in a subject in need thereof, comprising administering a therapeutically effective amount of a compound of Formula III:

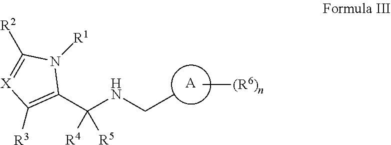

or a pharmaceutically acceptable salt thereof, wherein:
Ring A is selected from null, C₃₋₁₀carbocyclyl, 3- to 10-membered heterocyclyl, C₆₋₁₀aryl, and 5- to 10-membered heteroaryl;
X is selected from N and CR⁷;
R¹ is selected from H, C₁₋₈alkyl, C₂₋₈alkenyl, C₂₋₈alkynyl, C₁₋₈haloalkyl, C₁₋₈heteroalkyl, C₃₋₁₀carbocyclyl, 3- to 10-membered heterocyclyl, C₆₋₁₀aryl, and 5- to 10-membered heteroaryl;
R², R³, and R⁷ are independently selected from H, halo, hydroxy, amino, cyano, nitro, C₁₋₈alkyl, C₂₋₈alkenyl, C₋₈alkynyl, C₁₋₈haloalkyl, C₁₋₈heteroalkyl, C₁₋₈alkoxy, C₃₋₁₀carbocyclyl, 3- to 10-membered heterocyclyl, C₆₋₁₀aryl, and 5- to 10-membered heteroaryl;
R⁴ and R⁵ are independently selected from H, halo, cyano, nitro, C₁₋₈alkyl, C₂₋₈alkenyl, C₂₋₈alkynyl, C₁₋₈haloalkyl, C₁₋₈heteroalkyl, C₃₋₁₀carbocyclyl, 3- to 10-membered heterocyclyl, C₆₋₁₀aryl, and 5- to 10-membered heteroaryl; or
R⁴ and R⁵ are taken together to form oxo;
each R⁶ is independently selected from H, halo, hydroxy, amino, cyano, nitro, C₁₋₈alkyl, C₂₋₈alkenyl, C₂₋₈alkynyl, C₁₋₈haloalkyl, C₁₋₈heteroalkyl, C₁₋₈alkoxy, C₃₋₁₀carbocyclyl, 3- to 10-membered heterocyclyl, C₆₋₁₀aryl, and 5- to 10-membered heteroaryl; and
n is 0, 1, 2, 3, 4, 5, or 6.

In some embodiments, Ring A is selected from null, C₃₋₁₀carbocyclyl, 3- to 10-membered heterocyclyl, C₆₋₁₀aryl, and 5- to 10-membered heteroaryl. In some embodiments, Ring A is null. In some embodiments, Ring A is C₃₋₁₀carbocyclyl. In some embodiments, Ring A is 3- to 10-membered heterocyclyl. In some embodiments, Ring A is C₆₋₁₀aryl. In some embodiments, Ring A is 5- to 10-membered heteroaryl.

In some embodiments, X is selected from N and CR⁷. In some embodiments, X is N. In some embodiments, X is CR⁷.

In some embodiments, R¹ is selected from H, C₁₋₈alkyl, C₂₋₈alkenyl, C₂₋₈alkynyl, C₁₋₈haloalkyl, C₁₋₈heteroalkyl, C₃₋₁₀carbocyclyl, 3- to 10-membered heterocyclyl, C₆₋₁₀aryl, and 5- to 10-membered heteroaryl. In some embodiments, R¹ is selected from H, C₁₋₈alkyl, C₁₋₈haloalkyl, C₁₋₈heteroalkyl, C₃₋₁₀carbocyclyl, 3- to 10-membered heterocyclyl, C₆₋₁₀aryl, and 5- to 10-membered heteroaryl. In some embodiments, R¹ is selected from H, C₁₋₈alkyl, C₁₋₈haloalkyl, and C₁₋₈heteroalkyl. In some embodiments, R¹ is H. In some embodiments, R¹ is C₁₋₈alkyl. In some embodiments, R¹ is C₂₋₈alkenyl. In some embodiments, R¹ is C₂₋₈alkynyl. In some embodiments, R¹ is C₁₋₈haloalkyl. In some embodiments, R¹ is C₁₋₈heteroalkyl. In some embodiments, R¹ is C₃₋₁₀carbocyclyl. In some embodiments, R¹ is 3- to 10-membered heterocyclyl. In some embodiments, R¹ is C₆₋₁₀aryl. In some embodiments, R¹ is 5- to 10-membered heteroaryl.

In some embodiments, R², R³, and R 7 are independently selected from H, halo, hydroxy, amino, cyano, nitro, C₁₋₈alkyl, C₂₋₈alkenyl, C₂₋₈alkynyl, C₁₋₈haloalkyl, C₁₋₈heteroalkyl, C₁₋₈alkoxy, C₃₋₁₀carbocyclyl, 3- to 10-membered heterocyclyl, C₆₋₁₀aryl, and 5- to 10-membered heteroaryl. In some embodiments, R², R³, and R⁷ are independently selected from H, halo, hydroxy, amino, cyano, nitro, C₁₋₈alkyl, C₁₋₈haloalkyl, C₁₋₈heteroalkyl, C₁₋₈alkoxy, C₃₋₁₀carbocyclyl, 3- to 10-membered heterocyclyl, C₆₋₁₀aryl, and 5- to 10-membered heteroaryl. In some embodiments, R², R³, and R⁷ are independently selected from H, halo, hydroxy, amino, cyano, nitro, C₁₋₈alkyl, C₁₋₈haloalkyl, C₁₋₈heteroalkyl, and C₁₋₈alkoxy. In some embodiments, R², R³, and R⁷ are independently selected from H, halo, hydroxy, amino, cyano, and nitro. In some embodiments, R² is H. In some embodiments, R² is halo. In some embodiments, R² is hydroxy. In some embodiments, R² is amino. In some embodiments, R² is cyano. In some embodiments, R² is nitro. In some embodiments, R² is C₁₋₈alkyl. In some embodiments, R² is C₂₋₈alkenyl. In some embodiments, R² is C₂₋₈alkynyl. In some embodiments, R² is C₁₋₈haloalkyl. In some embodiments, R² is C₁₋₈heteroalkyl. In some embodiments, R² is C₁₋₈alkoxy. In some embodiments, R² is C₃₋₁₀carbocyclyl. In some embodiments, R² is 3- to 10-membered heterocyclyl. In some embodiments, R² is C₆₋₁₀aryl. In some embodiments, R² is 5- to 10-membered heteroaryl. In some embodiments, R³ is H. In some embodiments, R³ is halo. In some embodiments, R³ is hydroxy. In some embodiments, R³ is amino. In some embodiments, R³ is cyano. In some embodiments, R³ is nitro. In some embodiments, R³ is C₁₋₈alkyl. In some embodiments, R³ is C₂₋₈alkenyl. In some embodiments, R³ is C₂₋₈alkynyl. In some embodiments, R³ is C₁₋₈haloalkyl. In some embodiments, R³ is C₁₋₈heteroalkyl. In some embodiments, R³ is C₁₋₈alkoxy. In some embodiments, R³ is C₃₋₁₀carbocyclyl. In some embodiments, R³ is 3- to 10-membered heterocyclyl. In some embodiments, R³ is C₆₋₁₀aryl. In some embodiments, R³ is 5- to 10-membered heteroaryl. In some embodiments, R⁷ is H. In some embodiments, R⁷ is halo. In some embodiments, R⁷ is hydroxy. In some embodiments, R⁷ is amino. In some embodiments, R⁷ is cyano. In some embodiments, R⁷ is nitro. In some embodiments, R⁷ is C₁₋₈alkyl. In some embodiments, R⁷ is C₂₋₈alkenyl. In some embodiments, R⁷ is C₂₋₈alkynyl. In some embodiments, R⁷ is C₁₋₈haloalkyl. In some embodiments, R⁷ is C₁₋₈heteroalkyl. In some embodiments, R⁷ is C₁₋₈alkoxy. In some embodiments, R⁷ is C₃₋₁₀carbocyclyl. In some embodiments, R⁷ is 3- to 10-membered heterocyclyl. In some embodiments, R⁷ is C₆₋₁₀aryl. In some embodiments, R⁷ is 5- to 10-membered heteroaryl.

In some embodiments, R⁴ and R⁵ are independently selected from H, halo, cyano, nitro, C₁₋₈alkyl, C₂₋₈alkenyl, C₂₋₈alkynyl, C₁₋₈haloalkyl, C₁₋₈heteroalkyl, C₃₋₁₀carbocyclyl, 3- to 10-membered heterocyclyl, C₆₋₁₀aryl, and 5- to 10-membered heteroaryl; or R⁴ and R⁵ are taken together to form oxo. In some embodiments, R⁴ and R⁵ are taken together to form oxo. In some embodiments, R⁴ and R⁵ are independently selected from H, halo, cyano, nitro, C₁₋₈alkyl, C₂₋₈alkenyl, C₂₋₈alkynyl, C₁₋₈haloalkyl, C₁₋₈heteroalkyl, C₃₋₁₀carbocyclyl, 3- to 10-membered heterocyclyl, C₆₋₁₀aryl, and 5- to 10-membered heteroaryl. In some embodiments, R⁴ and R⁵ are independently selected from H, halo, cyano, nitro, C₁₋₈alkyl, C₁₋₈haloalkyl, C₁₋₈heteroalkyl, C₃₋₁₀carbocyclyl, 3- to 10-membered heterocyclyl, C₆₋₁₀aryl, and 5- to 10-membered heteroaryl. In some embodiments, R⁴ and R⁵ are independently selected from H, halo, cyano, nitro, C₁₋₈alkyl, C₁₋₈haloalkyl, and C₁₋₈heteroalkyl. In some embodiments, R⁴ and R⁵ are independently selected from H, halo, cyano, and nitro. In some embodiments, R⁴ is H. In some embodiments, R⁴ is halo. In some embodiments, R⁴ is cyano. In some embodiments, R⁴ is nitro. In some embodiments, R⁴ is C₁₋₈alkyl. In some embodiments, R⁴ is C₂₋₈alkenyl. In some embodiments, R⁴ is C₂₋₈alkynyl. In some embodiments, R⁴ is C₁₋₈haloalkyl. In some embodiments, R⁴ is C₁₋₈heteroalkyl. In some embodiments, R⁴ is C₃₋₁₀carbocyclyl. In some embodiments, R⁴ is 3- to 10-membered heterocyclyl. In some embodiments, R⁴ is C₆₋₁₀aryl. In some embodiments, R⁴ is 5- to 10-membered heteroaryl. In some embodiments, R⁵ is H. In some embodiments, R⁵ is halo. In some embodiments, R⁵ is cyano. In some embodiments, R⁵ is nitro. In some embodiments, R⁵ is C₁₋₈alkyl. In some embodiments, R⁵ is C₂₋₈alkenyl. In some embodiments, R⁵ is C₂₋₈alkynyl. In some embodiments, R⁵ is C₁₋₈haloalkyl. In some embodiments, R⁵ is C₁₋₈heteroalkyl. In some embodiments, R⁵ is C₃₋₁₀carbocyclyl. In some embodiments, R⁵ is 3- to 10-membered heterocyclyl. In some embodiments, R⁵ is C₆₋₁₀aryl. In some embodiments, R⁵ is 5- to 10-membered heteroaryl.

In some embodiments, each R⁶ is independently selected from H, halo, hydroxy, amino, cyano, nitro, C₁₋₈alkyl, C₂₋₈alkenyl, C₂₋₈alkynyl, C₁₋₈haloalkyl, C₁₋₈heteroalkyl, C₁₋₈alkoxy, C₃₋₁₀carbocyclyl, 3- to 10-membered heterocyclyl, C₆₋₁₀aryl, and 5- to 10-membered heteroaryl. In some embodiments, each R⁶ is independently selected from H, halo, hydroxy, amino, cyano, nitro, C₁₋₈alkyl, C₁₋₈haloalkyl, C₁₋₈heteroalkyl, C₁₋₈alkoxy, C₃₋₁₀carbocyclyl, 3- to 10-membered heterocyclyl, C₆₋₁₀aryl, and 5- to 10-membered heteroaryl. In some embodiments, each R⁶ is independently selected from H, halo, hydroxy, amino, cyano, nitro, C₁₋₈alkyl, C₁₋₈haloalkyl, C₁₋₈heteroalkyl, and C₁₋₈alkoxy. In some embodiments, each R⁶ is independently selected from H, halo, hydroxy, amino, cyano, and nitro. In some embodiments, each R⁶ is independently H. In some embodiments, each R⁶ is independently halo. In some embodiments, each R⁶ is independently hydroxy. In some embodiments, each R⁶ is independently amino. In some embodiments, each R⁶ is independently cyano. In some embodiments, each R⁶ is independently nitro. In some embodiments, each R⁶ is independently C₁₋₈alkyl. In some embodiments, each R⁶ is independently C₂₋₈alkenyl. In some embodiments, each R⁶ is independently C₂₋₈alkynyl. In some embodiments, each R⁶ is independently C₁₋₈haloalkyl. In some embodiments, each R⁶ is independently C₁₋₈heteroalkyl. In some embodiments, each R⁶ is independently C₁₋₈alkoxy. In some embodiments, each R⁶ is independently C₃₋₁₀carbocyclyl. In some embodiments, each R⁶ is independently 3- to 10-membered heterocyclyl. In some embodiments, each R⁶ is independently C₆₋₁₀aryl. In some embodiments, each R⁶ is independently 5- to 10-membered heteroaryl.

In some embodiments, n is 0, 1, 2, 3, 4, 5, or 6. In some embodiments, n is 0. In some embodiments, n is 1. In some embodiments, n is 2. In some embodiments, n is 3. In some embodiments, n is 4. In some embodiments, n is 5. In some embodiments, n is 6.

In some embodiments, provided herein is a compound shown in Table 1.

TABLE 1
Exemplary Compounds

| Cmpd. | | |
|---|---|---|
| No. | Structure | Name |
| CPD- 001 | 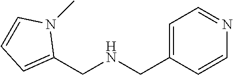 | 1-(1-methyl-1H-pyrrol- 2-yl)-N-(pyridin-4- ylmethyl)methanamine |
| CPD- 002 | 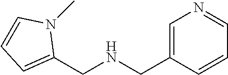 | 1-(1-methyl-1H-pyrrol- 2-yl)-N-(pyridin-3- ylmethyl)methanamine |
| CPD- 003 | 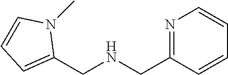 | 1-(1-methyl-1H-pyrrol- 2-yl)-N-(pyridin-2- ylmethyl)methanamine |
| CPD- 004 | 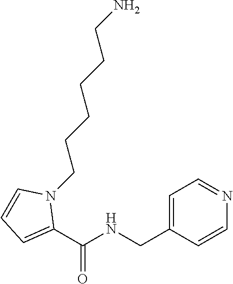 [SMILES: NCCCCCCn1cccc1C(=O)NCc1ccncc1] | 1-(6-aminohexyl)-N- (pyridin-4-ylmethyl)- 1H-pyrrole-2-carboxamide |
| CPD- 005 | 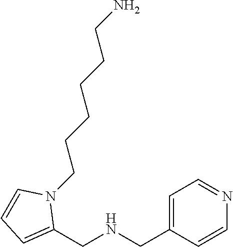 [SMILES: NCCCCCCn1cccc1CNCc1ccncc1] | 6-(2-(((pyridin-4- ylmethyl)amino) methyl)-1H-pyrrol-1- yl)hexan-1-amine |
| CPD- 006 | 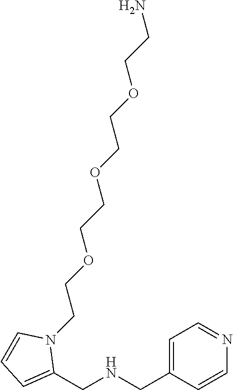 [SMILES: NCCOCCOCCOCCn1cccc1CNCc1ccncc1] | 2-(2-(2-(2-(2-(((pyridin-4- ylmethyl)amino) methyl)-1H-pyrrol-1- yl)ethoxy)ethoxy) ethoxy)ethan-1-amine |
| CPD- 007 | 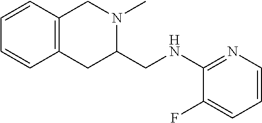 [SMILES: CN1Cc2ccccc2CC1CNc1ncccc1F] | 3-fluoro-N-((2-methyl-1,2,3,4- tetrahydroisoquinolin-3- yl)methyl)pyridin-2-amine |
| CPD- 008 | 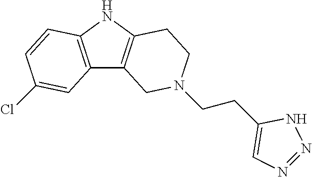 [SMILES: Clc1ccc2[nH]c3c(c2c1)CN(CCc1cnn[nH]1)CC3] | 2-(2-(1H-1,2,3- triazol-5-yl)ethyl)-8- chloro-2,3,4,5-tetrahydro- 1H-pyrido[4,3- b]indole |
| CPD- 009 | 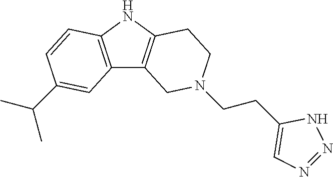 [SMILES: CC(C)c1ccc2[nH]c3c(c2c1)CN(CCc1cnn[nH]1)CC3] | 2-(2-(1H-1,2,3- triazol-5-yl)ethyl)-8- isopropyl-2,3,4,5-tetrahydro-1H- pyrido[4,3-b]indole |
| CPD- 010 | 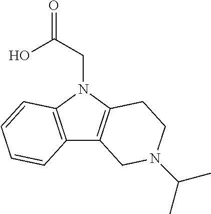 [SMILES: CC(C)N1CCc2c(c3ccccc3n2CC(=O)O)C1] | 2-(2-isopropyl-1,2,3,4- tetrahydro-5H- pyrido[4,3-b]indol- 5-yl)acetic acid |
| CPD- 011 | 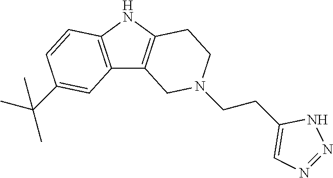 [SMILES: CC(C)(C)c1ccc2[nH]c3c(c2c1)CN(CCc1cnn[nH]1)CC3] | 2-(2-(1H-1,2,3-triazol- 5-yl)ethyl)-8-(tert- butyl)-2,3,4,5-tetrahydro- 1H-pyrido[4,3- b]indole |
| CPD- 012 | 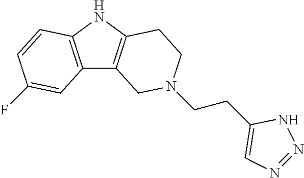 [SMILES: Fc1ccc2[nH]c3c(c2c1)CN(CCc1cnn[nH]1)CC3] | 2-(2-(1H-1,2,3- triazol-5-yl)ethyl)-8- fluoro-2,3,4,5-tetrahydro- 1H-pyrido[4,3- b]indole |
| CPD- 013 | 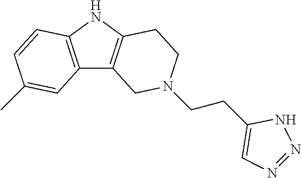 [SMILES: Cc1ccc2[nH]c3c(c2c1)CN(CCc1cnn[nH]1)CC3] | 2-(2-(1H-1,2,3- triazol-5-yl)ethyl)-8- methyl-2,3,4,5-tetrahydro- 1H-pyrido[4,3- b]indole |
| CPD- 014 | 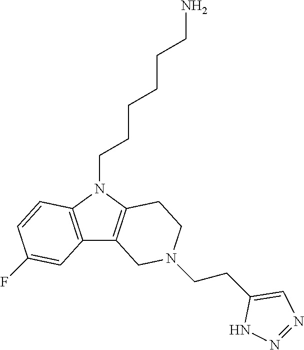 [SMILES: NCCCCCCn1c2c(c3cc(F)ccc31)CN(CCc1cnn[nH]1)CC2] | 6-(2-(2-(1H-1,2,3- triazol-5-yl)ethyl)-8- fluoro-1,2,3,4-tetrahydro- 5H-pyrido[4,3- b]indol-5-yl)hexan-1-amine |
| CPD- 015 | 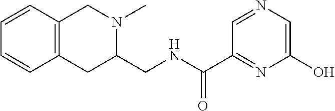 [SMILES: CN1Cc2ccccc2CC1CNC(=O)c1cncc(O)n1] | 6-hydroxy-N- ((2-methyl-1,2,3,4- tetrahydroisoquinolin-3- yl)methyl)pyrazine- 2-carboxamide |
| CPD- 016 | 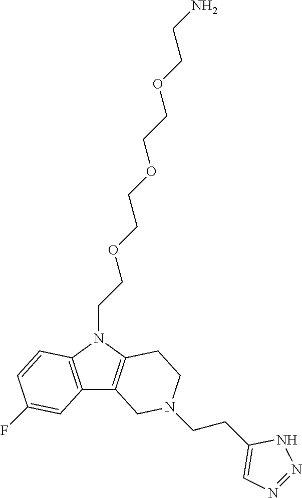 [SMILES: NCCOCCOCCOCCn1c2c(c3cc(F)ccc31)CN(CCc1cnn[nH]1)CC2] | 2-(2-(2-(2-(2-(2- (1H-1,2,3-triazol-5- yl)ethyl)-8-fluoro- 1,2,3,4-tetrahydro-5H- pyrido[4,3-b]indol-5- yl)ethoxy)ethoxy) ethoxy)ethan-1-amine |
| CPD- 017 | 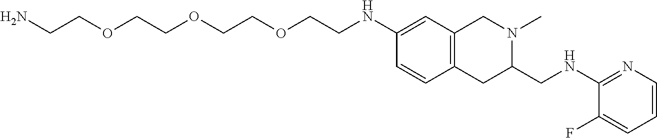 [SMILES: CN1Cc2cc(NCCOCCOCCOCCN)ccc2CC1CNc1ncccc1F] | N-(2-(2-(2-(2- aminoethoxy)ethoxy) ethoxy)ethyl)-3-(((3- fluoropyridin-2-yl) amino)methyl)-2- methyl-1,2,3,4- tetrahydroisoquinolin-7- amine |
| CPD- 018 | 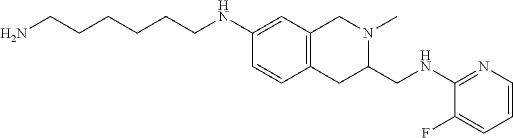 [SMILES: CN1Cc2cc(NCCCCCCN)ccc2CC1CNc1ncccc1F] | N1-(3-(((3- fluoropyridin-2- yl)amino)methyl)- 2-methyl-1,2,3,4- tetrahydroisoquinolin- 7-yl)hexane-1,6- diamine |
| CPD- 019 | 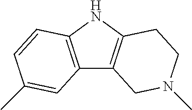 | 2,8-dimethyl- 2,3,4,5-tetrahydro-1H- pyrido[4,3-b]indole |
| CPD- 020 | 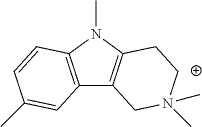 | 2,2,5,8-tetramethyl- 2,3,4,5-tetrahydro-1H- 2λ⁴-pyrido[4,3-b]indol-3-ylium |
| CPD- 021 | 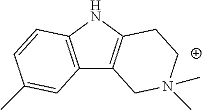 | 2,2,8-trimethyl-2,3,4,5- tetrahydro-1H-2λ⁴- pyrido[4,3-b]indol-3-ylium |
| CPD- 022 | 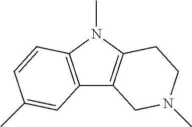 | 2,5,8-trimethyl- 2,3,4,5-tetrahydro-1H- pyrido[4,3-b]indole |
| CPD- 023 | 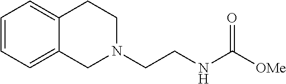 [SMILES: COC(=O)NCCN1CCc2ccccc2C1] | methyl (2-(3,4- dihydroisoquinolin-2(1H)- yl)ethyl)carbamate |
| CPD- 024 | 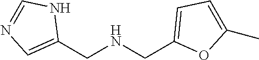 | N-((1H-imidazol- 5-yl)methyl)-1-(5- methylfuran-2-yl) methanamine |
| CPD- 025 | 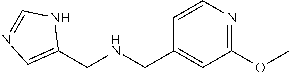 [SMILES: COc1cc(CNCc2cnc[nH]2)ccn1] | N-((1H-imidazol- 5-yl)methyl)-1-(2- methoxypyridin-4-yl) methanamine |
| CPD- 026 | 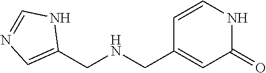 [SMILES: O=c1cc(CNCc2cnc[nH]2)cc[nH]1] | 4-((((1H-imidazol-5- yl)methyl)amino) methyl)pyridin-2(1H)- one |
| CPD- 027 | 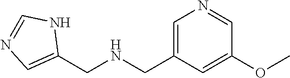 [SMILES: COc1cncc(CNCc2cnc[nH]2)c1] | N-((1H-imidazol- 5-yl)methyl)-1-(5- methoxypyridin-3-yl) methanamine |
| CPD- 028 | 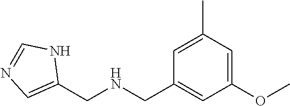 [SMILES: COc1cc(C)cc(CNCc2cnc[nH]2)c1] | N-((1H-imidazol- 5-yl)methyl)-1-(3- methoxy-5-methylphenyl) methanamine |
| CPD- 029 | 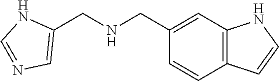 [SMILES: c1ncc(CNCc2ccc3cc[nH]c3c2)[nH]1] | N-((1H-imidazol-5- yl)methyl)-1-(1H- indol-6-yl)methanamine |
| CPD- 030 | 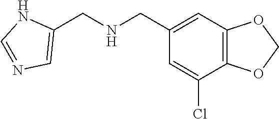 [SMILES: Clc1cc(CNCc2cnc[nH]2)cc2c1OCO2] | N-((1H-imidazol- 5-yl)methyl)-1-(7- chlorobenzo[d][1,3]dioxol-5- yl)methanamine |
| CPD- 031 | 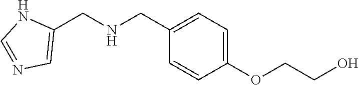 [SMILES: OCCOc1ccc(CNCc2cnc[nH]2)cc1] | 2-(4-((((1H-imidazol-5- yl)methyl)amino) methyl)phenoxy)ethan- 1-ol |
| CPD- 032 | 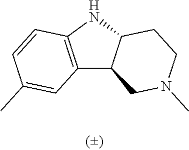 | trans-2,8-dimethyl- 2,3,4,4a,5,9b- hexahydro-1H-pyrido [4,3-b]indole |
| CPD- 033 | 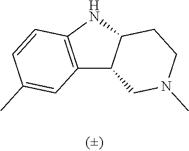 | cis-2,8-dimethyl- 2,3,4,4a,5,9b-hexahydro- 1H-pyrido[4,3-b]indole |
| CPD- 034 | 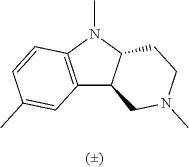 | trans-2,5,8- trimethyl-2,3,4,4a,5,9b- hexahydro-1H- pyrido[4,3-b]indole |
| CPD- 035 | 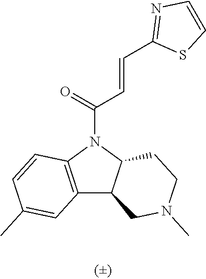 [SMILES: Cc1ccc2c(c1)[C@@H]1CN(C)CC[C@H]1N2C(=O)/C=C/c1nccs1] | (E)-1-(trans-2,8- dimethyl-1,2,3,4,4a,9b- hexahydro-5H-pyrido [4,3-b]indol-5-yl)-3- (thiazol-2-yl)prop-2-en-1-one |
| CPD- 036 | 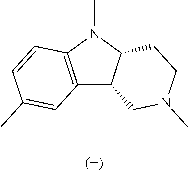 | cis-2,5,8-trimethyl- 2,3,4,4a,5,9b- hexahydro-1H-pyrido [4,3-b]indole |
| CPD- 037 | 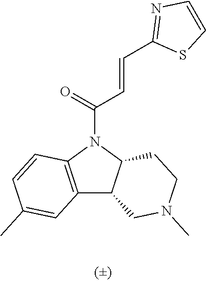 [SMILES: Cc1ccc2c(c1)[C@H]1CN(C)CC[C@H]1N2C(=O)/C=C/c1nccs1] | (E)-1-(cis-2,8- dimethyl-1,2,3,4,4a,9b- hexahydro-5H-pyrido [4,3-b]indol-5-yl)-3- (thiazol-2-yl)prop-2-en-1-one |
| CPD- 038 | 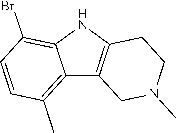 [SMILES: Cc1ccc(Br)c2[nH]c3c(c12)CN(C)CC3] | 6-bromo-2,9-dimethyl- 2,3,4,5-tetrahydro- 1H-pyrido[4,3-b]indole |
| CPD- 039 | 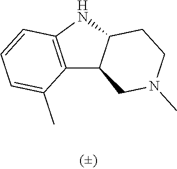 | trans-2,9-dimethyl- 2,3,4,4a,5,9b- hexahydro-1H-pyrido [4,3-b]indole |
| CPD- 040 | 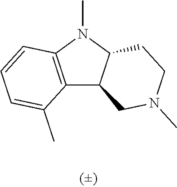 [SMILES: Cc1cccc2c1[C@@H]1CN(C)CC[C@H]1N2C] | trans-2,5,9-trimethyl- 2,3,4,4a,5,9b- hexahydro-1H- pyrido[4,3-b]indole |
| CPD- 041 | 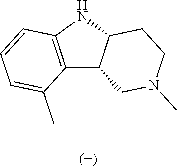 | cis-2,9-dimethyl- 2,3,4,4a,5,9b-hexahydro- 1H-pyrido[4,3-b]indole |
| CPD- 042 | 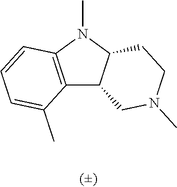 [SMILES: Cc1cccc2c1[C@H]1CN(C)CC[C@H]1N2C] | cis-2,5,9-trimethyl- 2,3,4,4a,5,9b- hexahydro-1H- pyrido[4,3-b]indole |
| CPD- 043 | 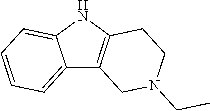 | 2-ethyl-2,3,4,5- tetrahydro-1H-pyrido[4,3- b]indole |
| CPD- 044 | 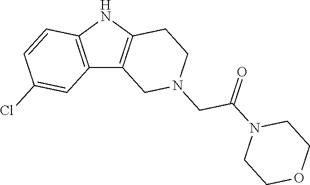 [SMILES: O=C(CN1CCc2[nH]c3ccc(Cl)cc3c2C1)N1CCOCC1] | 2-(8-chloro-1,3,4,5- tetrahydro-2H- pyrido[4,3-b]indol-2-yl)-1- morpholinoethan-1-one |
| CPD- 045 | 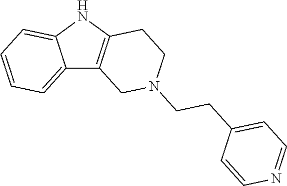 [SMILES: c1ccc2c3c([nH]c2c1)CCN(CCc1ccncc1)C3] | 2-(2-(pyridin-4- yl)ethyl)-2,3,4,5- tetrahydro-1H- pyrido[4,3-b]indole |
| CPD- 046 | 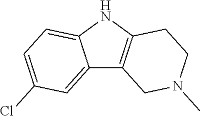 | 8-chloro-2-methyl- 2,3,4,5-tetrahydro-1H- pyrido[4,3-b]indole |
| CPD- 047 | 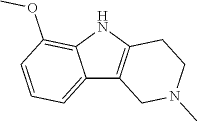 | 6-methoxy-2- methyl-2,3,4,5-tetrahydro- 1H-pyrido[4,3-b]indole |
| CPD- 048 | 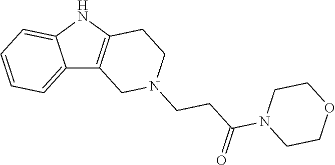 [SMILES: O=C(CCN1CCc2[nH]c3ccccc3c2C1)N1CCOCC1] | 1-morpholino-3- (1,3,4,5-tetrahydro-2H- pyrido[4,3-b]indol- 2-yl)propan-1-one |
| CPD- 049 | 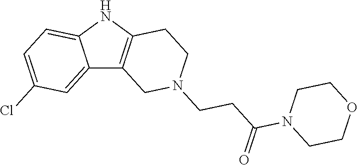 [SMILES: O=C(CCN1CCc2[nH]c3ccc(Cl)cc3c2C1)N1CCOCC1] | 3-(8-chloro- 1,3,4,5-tetrahydro-2H- pyrido[4,3-b]indol-2-yl)-1- morpholinopropan-1-one |
| CPD- 050 | 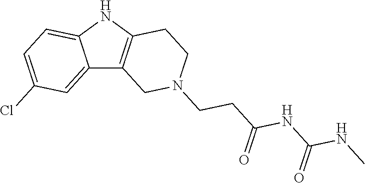 [SMILES: CNC(=O)NC(=O)CCN1CCc2[nH]c3ccc(Cl)cc3c2C1] | 3-(8-chloro- 1,3,4,5-tetrahydro-2H- pyrido[4,3-b]indol-2-yl)-N- (methylcarbamoyl)propanamide |
| CPD- 051 | 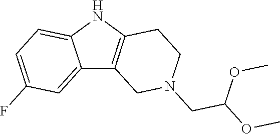 [SMILES: COC(CN1CCc2[nH]c3ccc(F)cc3c2C1)OC] | 2-(2,2-dimethoxyethyl)- 8-fluoro-2,3,4,5- tetrahydro-1H- pyrido[4,3-b]indole |
| CPD- 052 | 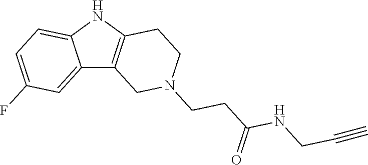 [SMILES: C#CCNC(=O)CCN1CCc2[nH]c3ccc(F)cc3c2C1] | 3-(8-fluoro- 1,3,4,5-tetrahydro-2H- pyrido[4,3-b]indol-2- yl)-N-(prop-2-yn-1- yl)propanamide |
| CPD- 053 | 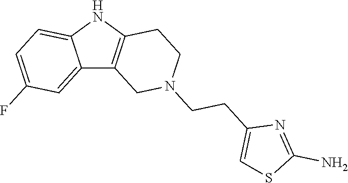 [SMILES: Nc1nc(CCN2CCc3[nH]c4ccc(F)cc4c3C2)cs1] | 4-(2-(8-fluoro- 1,3,4,5-tetrahydro-2H- pyrido[4,3-b]indol-2- yl)ethyl)thiazol-2- amine |
| CPD- 054 | 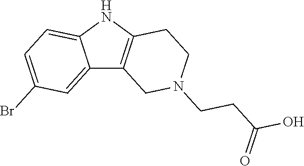 [SMILES: O=C(O)CCN1CCc2[nH]c3ccc(Br)cc3c2C1] | 3-(8-bromo- 1,3,4,5-tetrahydro-2H- pyrido[4,3-b]indol- 2-yl)propanoic acid |
| CPD- 055 | 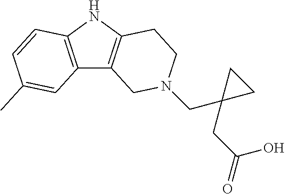 [SMILES: Cc1ccc2[nH]c3c(c2c1)CN(CC1(CC(=O)O)CC1)CC3] | 2-(1-((8-methyl- 1,3,4,5-tetrahydro-2H- pyrido[4,3-b]indol-2- yl)methyl)cyclopropyl) acetic acid |
| CPD- 056 | 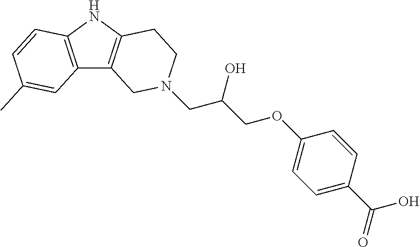 [SMILES: Cc1ccc2[nH]c3c(c2c1)CN(CC(O)COc1ccc(C(=O)O)cc1)CC3] | 4-(2-hydroxy-3- (8-methyl-1,3,4,5- tetrahydro-2H- pyrido[4,3-b]indol-2- yl)propoxy)benzoic acid |
| CPD- 057 | 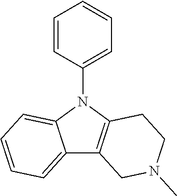 [SMILES: CN1CCc2c(c3ccccc3n2-c2ccccc2)C1] | 2-methyl-5-phenyl- 2,3,4,5-tetrahydro-1H- pyrido[4,3-b]indole |
| CPD- 058 | 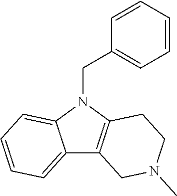 [SMILES: CN1CCc2c(c3ccccc3n2Cc2ccccc2)C1] | 5-benzyl-2-methyl- 2,3,4,5-tetrahydro-1H- pyrido[4,3-b]indole |
| CPD- 059 | 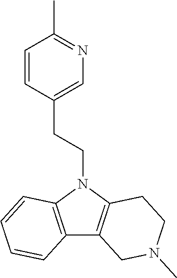 [SMILES: Cc1ccc(CCn2c3c(c4ccccc42)CN(C)CC3)cn1] | 2-methyl-5-(2- (6-methylpyridin-3- yl)ethyl)-2,3,4,5- tetrahydro-1H- pyrido[4,3-b]indole |
| CPD- 060 | 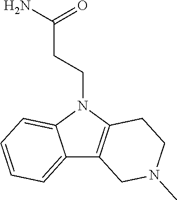 [SMILES: CN1CCc2c(c3ccccc3n2CCC(N)=O)C1] | 3-(2-methyl- 1,2,3,4-tetrahydro-5H- pyrido[4,3-b]indol- 5-yl)propanamide |
| CPD- 061 | 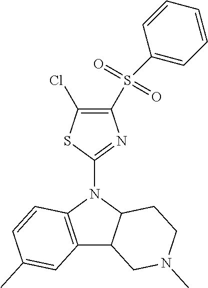 [SMILES: Cc1ccc2c(c1)C1CN(C)CCC1N2c1nc(S(=O)(=O)c2ccccc2)c(Cl)s1] | 5-chloro-2-(2,8- dimethyl-1,2,3,4,4a,9b- hexahydro-5H-pyrido [4,3-b]indol-5-yl)-4- (phenylsulfonyl)thiazole |
| CPD- 062 | 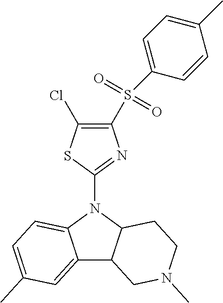 | 5-chloro-2-(2,8- dimethyl-1,2,3,4,4a,9b- hexahydro-5H-pyrido [4,3-b]indol-5-yl)-4- tosylthiazole |
| CPD- 063 | 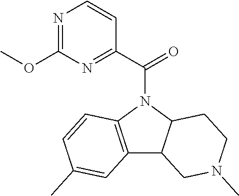 | (2,8-dimethyl- 1,2,3,4,4a,9b-hexahydro- 5H-pyrido[4,3-b] indol-5-yl)(2- methoxypyrimidin- 4-yl)methanone |
| CPD- 064 | 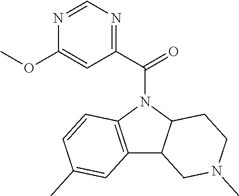 | (2,8-dimethyl- 1,2,3,4,4a,9b-hexahydro- 5H-pyrido [4,3-b]indol-5-yl)(6- methoxypyrimidin- 4-yl)methanone |
| CPD- 065 | 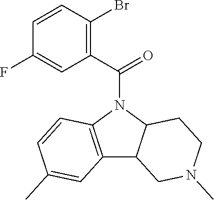 [SMILES: Cc1ccc2c(c1)C1CN(C)CCC1N2C(=O)c1cc(F)ccc1Br] | (2-bromo-5-fluorophenyl) (2,8-dimethyl- 1,2,3,4,4a,9b-hexahydro- 5H-pyrido[4,3- b]indol-5-yl)methanone |
| CPD- 066 | 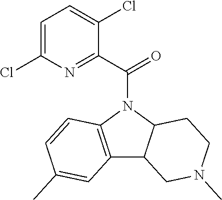 [SMILES: Cc1ccc2c(c1)C1CN(C)CCC1N2C(=O)c1nc(Cl)ccc1Cl] | (3,6-dichloropyridin-2-yl) (2,8-dimethyl- 1,2,3,4,4a,9b-hexahydro- 5H-pyrido[4,3- b]indol-5-yl)methanone |
| CPD- 067 | 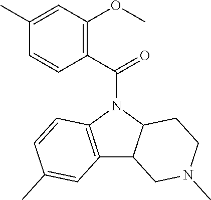 | (2,8-dimethyl- 1,2,3,4,4a,9b-hexahydro- 5H-pyrido[4,3-b]indol- 5-yl)(2-methoxy-4- methylphenyl)methanone |
| CPD- 068 | 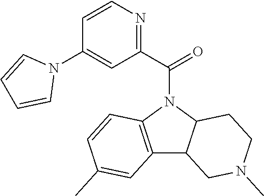 [SMILES: Cc1ccc2c(c1)C1CN(C)CCC1N2C(=O)c1cc(-n2cccc2)ccn1] | (4-(1H-pyrrol-1- yl)pyridin-2-yl)(2,8- dimethyl-1,2,3,4,4a,9b- hexahydro-5H- pyrido[4,3-b]indol- 5-yl)methanone |
| CPD- 069 | 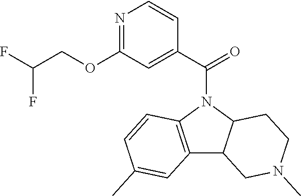 [SMILES: Cc1ccc2c(c1)C1CN(C)CCC1N2C(=O)c1ccnc(OCC(F)F)c1] | (2-(2,2-difluoroethoxy) pyridin-4-yl)(2,8- dimethyl-1,2,3,4,4a,9b- hexahydro-5H- pyrido[4,3-b]indol- 5-yl)methanone |
| CPD- 070 | 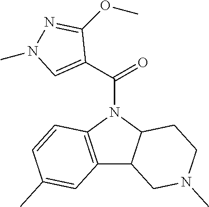 | (2,8-dimethyl- 1,2,3,4,4a,9b-hexahydro- 5H-pyrido[4,3-b]indol- 5-yl)(3-methoxy-1- methyl-1H-pyrazol- 4-yl)methanone |
| CPD- 071 | 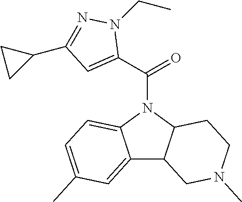 [SMILES: CCn1nc(C2CC2)cc1C(=O)N1c2ccc(C)cc2C2CN(C)CCC21] | (3-cyclopropyl-1- ethyl-1H-pyrazol-5- yl)(2,8-dimethyl- 1,2,3,4,4a,9b-hexahydro- 5H-pyrido[4,3-b]indol- 5-yl)methanone |
| CPD- 072 | 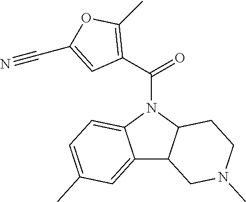 [SMILES: Cc1ccc2c(c1)C1CN(C)CCC1N2C(=O)c1cc(C#N)oc1C] | 4-(2,8-dimethyl- 2,3,4,4a,5,9b-hexahydro- 1H-pyrido[4,3-b]indole- 5-carbonyl)-5- methylfuran-2-carbonitrile |
| CPD- 073 | 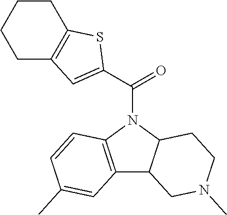 [SMILES: Cc1ccc2c(c1)C1CN(C)CCC1N2C(=O)c1cc2c(s1)CCCC2] | (2,8-dimethyl- 1,2,3,4,4a,9b-hexahydro- 5H-pyrido[4,3-b] indol-5-yl)(4,5,6,7- tetrahydrobenzo[b]thiophen-2- yl)methanone |
| CPD- 074 | 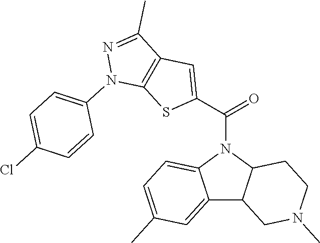 [SMILES: Cc1ccc2c(c1)C1CN(C)CCC1N2C(=O)c1cc2c(C)nn(-c3ccc(Cl)cc3)c2s1] | (1-(4-chlorophenyl)- 3-methyl-1H- thieno[2,3-c]pyrazol- 5-yl)(2,8-dimethyl- 1,2,3,4,4a,9b-hexahydro- 5H-pyrido[4,3- b]indol-5-yl)methanone |
| CPD- 075 | 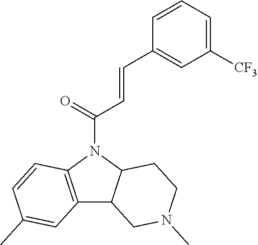 [SMILES: Cc1ccc2c(c1)C1CN(C)CCC1N2C(=O)/C=C/c1cccc(C(F)(F)F)c1] | (E)-1-(2,8-dimethyl- 1,2,3,4,4a,9b- hexahydro-5H-pyrido [4,3-b]indol-5-yl)-3- (3-(trifluoromethyl) phenyl)prop-2-en-1- one |
| CPD- 076 | 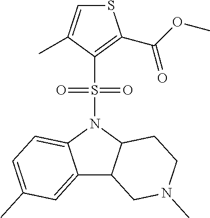 | methyl 3-((2,8- dimethyl-1,2,3,4,4a,9b- hexahydro-5H- pyrido[4,3-b]indol-5- yl)sulfonyl)-4- methylthiophene-2- carboxylate |
| CPD- 077 | 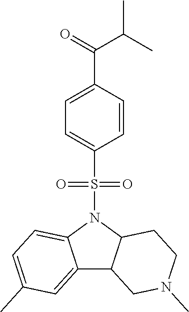 | 1-(4-((2,8-dimethyl- 1,2,3,4,4a,9b- hexahydro-5H- pyrido[4,3-b]indol-5- yl)sulfonyl)phenyl)-2- methylpropan-1-one |
| CPD- 078 | 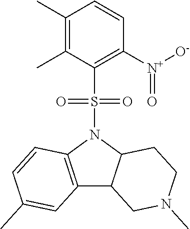 | 5-((2,3-dimethyl-6- nitrophenyl)sulfonyl)- 2,8-dimethyl-2,3,4,4a,5,9b- hexahydro-1H- pyrido[4,3-b]indole |
| CPD- 079 | 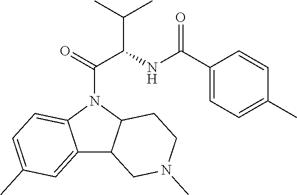 | N-((2S)-1-(2,8- dimethyl-1,2,3,4,4a,9b- hexahydro-5H-pyrido [4,3-b]indol-5-yl)-3- methyl-1-oxobutan-2-yl)-4- methylbenzamide |
| CPD- 080 | 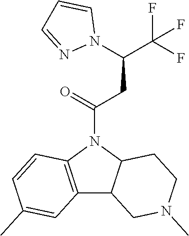 | (R)-1-(2,8-dimethyl- 1,2,3,4,4a,9b- hexahydro-5H-pyrido [4,3-b]indol-5-yl)- 4,4,4-trifluoro-3-(1H- pyrazol-1-yl)butan- 1-one |
| CPD- 081 | 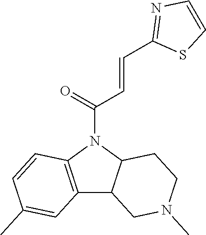 [SMILES: Cc1ccc2c(c1)C1CN(C)CCC1N2C(=O)/C=C/c1nccs1] | (E)-1-(2,8-dimethyl- 1,2,3,4,4a,9b- hexahydro-5H-pyrido [4,3-b]indol-5-yl)-3- (thiazol-2-yl)prop-2-en-1-one |
| CPD- 082 | 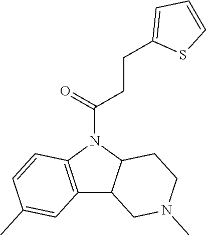 [SMILES: Cc1ccc2c(c1)C1CN(C)CCC1N2C(=O)CCc1cccs1] | 1-(2,8-dimethyl- 1,2,3,4,4a,9b-hexahydro- 5H-pyrido[4,3-b]indol- 5-yl)-3-(thiophen- 2-yl)propan-1-one |
| CPD- 083 | 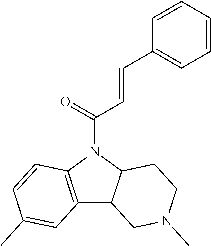 [SMILES: Cc1ccc2c(c1)C1CN(C)CCC1N2C(=O)/C=C/c1ccccc1] | (E)-1-(2,8-dimethyl- 1,2,3,4,4a,9b- hexahydro-5H-pyrido [4,3-b]indol-5-yl)-3- phenylprop-2-en-1-one |
| CPD- 084 | 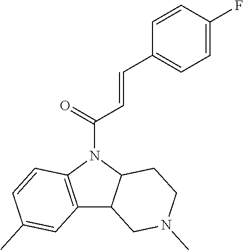 [SMILES: Cc1ccc2c(c1)C1CN(C)CCC1N2C(=O)/C=C/c1ccc(F)cc1] | (E)-1-(2,8-dimethyl- 1,2,3,4,4a,9b- hexahydro-5H-pyrido [4,3-b]indol-5-yl)-3- (4-fluorophenyl)prop-2- en-1-one |
| CPD- 085 | 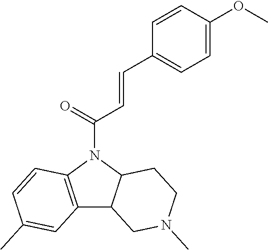 | (E)-1-(2,8-dimethyl- 1,2,3,4,4a,9b- hexahydro-5H-pyrido [4,3-b]indol-5-yl)-3- (4-methoxyphenyl)prop- 2-en-1-one |
| CPD- 086 | 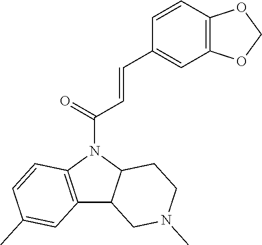 [SMILES: Cc1ccc2c(c1)C1CN(C)CCC1N2C(=O)/C=C/c1ccc2c(c1)OCO2] | (E)-3-(benzo[d][1,3] dioxol-5-yl)-1-(2,8- dimethyl-1,2,3,4,4a,9b- hexahydro-5H- pyrido[4,3-b]indol-5-yl) prop-2-en-1-one |
| CPD- 087 | 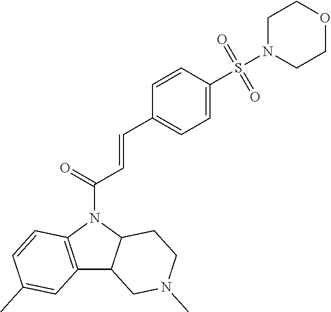 [SMILES: Cc1ccc2c(c1)C1CN(C)CCC1N2C(=O)/C=C/c1ccc(S(=O)(=O)N2CCOCC2)cc1] | (E)-1-(2,8-dimethyl- 1,2,3,4,4a,9b- hexahydro-5H-pyrido [4,3-b]indol-5-yl)-3- (4-(morpholinosulfonyl) phenyl)prop-2-en- 1-one |
| CPD- 088 | 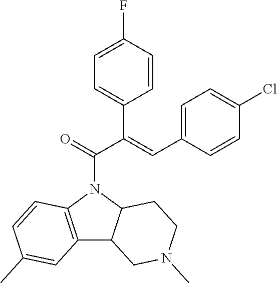 [SMILES: Cc1ccc2c(c1)C1CN(C)CCC1N2C(=O)/C(=C/c1ccc(Cl)cc1)c1ccc(F)cc1] | (E)-3-(4-chlorophenyl)- 1-(2,8-dimethyl- 1,2,3,4,4a,9b-hexahydro- 5H-pyrido[4,3- b]indol-5-yl)-2-(4- fluorophenyl)prop-2- en-1-one |
| CPD- 089 | 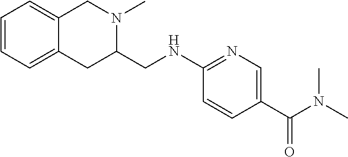 [SMILES: CN(C)C(=O)c1ccc(NCC2Cc3ccccc3CN2C)nc1] | N,N-dimethyl-6- (((2-methyl-1,2,3,4- tetrahydroisoquinolin-3- yl)methyl)amino)nicotinamide |
| CPD- 090 | 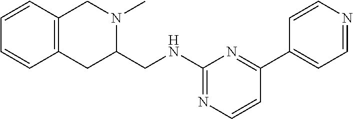 [SMILES: CN1Cc2ccccc2CC1CNc1nccc(-c2ccncc2)n1] | N-((2-methyl-1,2,3,4- tetrahydroisoquinolin- 3-yl)methyl)-4- (pyridin-4-yl)pyrimidin-2-amine |
| CPD- 091 | 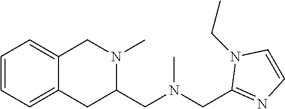 [SMILES: CCn1ccnc1CN(C)CC1Cc2ccccc2CN1C] | 1-(1-ethyl-1H-imidazol- 2-yl)-N-methyl-N- ((2-methyl-1,2,3,4- tetrahydroisoquinolin- 3-yl)methyl)methanamine |
| CPD- 092 | 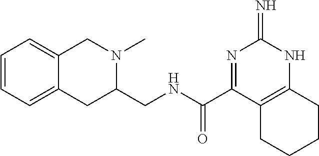 [SMILES: CN1Cc2ccccc2CC1CNC(=O)c1nc(=N)[nH]c2c1CCCC2] | 2-imino-N- ((2-methyl-1,2,3,4- tetrahydroisoquinolin- 3-yl)methyl)- 1,2,5,6,7,8- hexahydroquinazoline-4- carboxamide |
| CPD- 093 | 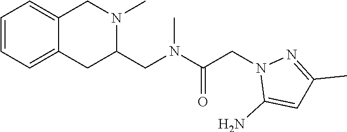 [SMILES: Cc1cc(N)n(CC(=O)N(C)CC2Cc3ccccc3CN2C)n1] | 2-(5-amino-3-methyl- 1H-pyrazol-1-yl)-N- methyl-N-((2-methyl-1,2,3,4- tetrahydroisoquinolin-3- yl)methyl)acetamide |
| CPD- 094 | 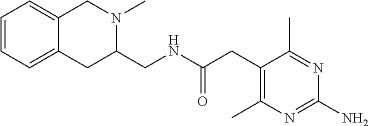 [SMILES: Cc1nc(N)nc(C)c1CC(=O)NCC1Cc2ccccc2CN1C] | 2-(2-amino-4,6- dimethylpyrimidin-5-yl)- N-((2-methyl-1,2,3,4- tetrahydroisoquinolin-3- yl)methyl)acetamide |
| CPD- 095 | 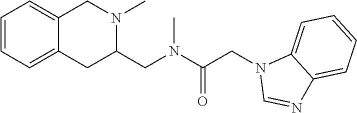 [SMILES: CN(CC1Cc2ccccc2CN1C)C(=O)Cn1cnc2ccccc21] | 2-(1H-benzo[d]imidazol- 1-yl)-N-methyl- N-((2-methyl-1,2,3,4- tetrahydroisoquinolin-3- yl)methyl)acetamide |
| CPD- 096 | 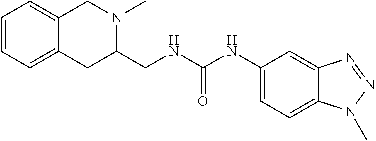 [SMILES: CN1Cc2ccccc2CC1CNC(=O)Nc1ccc2c(c1)nnn2C] | 1-((2-methyl-1,2,3,4- tetrahydroisoquinolin- 3-yl)methyl)-3-(1- methyl-1H-benzo [d][1,2,3]triazol-5- yl)urea |
| CPD- 097 | 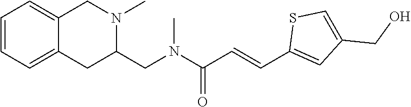 [SMILES: CN(CC1Cc2ccccc2CN1C)C(=O)/C=C/c1cc(CO)cs1] | (E)-3-(4-(hydroxymethyl) thiophen-2-yl)- N-methyl-N-((2-methyl-1,2,3,4- tetrahydroisoquinolin-3- yl)methyl)acrylamide |
| CPD- 098 | 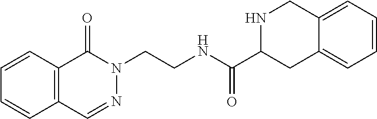 [SMILES: O=C(NCCn1ncc2ccccc2c1=O)C1Cc2ccccc2CN1] | N-(2-(1-oxophthalazin- 2(1H)-yl)ethyl)- 1,2,3,4-tetrahydroisoquinoline-3- carboxamide |
| CPD- 099 | 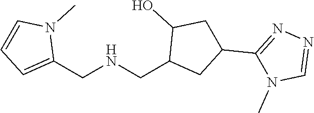 [SMILES: Cn1cccc1CNCC1CC(c2nncn2C)CC1O] | 2-((((1-methyl- 1H-pyrrol-2- yl)methyl)amino) methyl)-4-(4-methyl- 4H-1,2,4-triazol-3- yl)cyclopentan-1-ol |
| CPD- 100 | 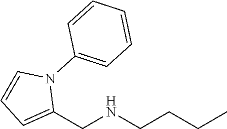 [SMILES: CCCCNCc1cccn1-c1ccccc1] | N-((1-phenyl-1H-pyrrol-2- yl)methyl)butan-1-amine |
| CPD- 101 | 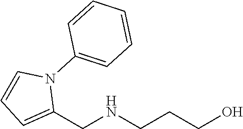 [SMILES: OCCCNCc1cccn1-c1ccccc1] | 3-(((1-phenyl-1H-pyrrol-2- yl)methyl)amino)propan-1-ol |
| CPD- 102 | 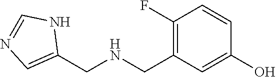 [SMILES: Oc1ccc(F)c(CNCc2cnc[nH]2)c1] | 3-((((1H-imidazol-5- yl)methyl)amino) methyl)-4-fluorophenol |
| CPD- 103 | 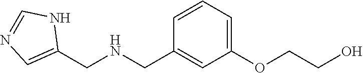 [SMILES: OCCOc1cccc(CNCc2cnc[nH]2)c1] | 2-(3-((((1H-imidazol-5- yl)methyl)amino) methyl)phenoxy)ethan- 1-ol |
| CPD- 104 | 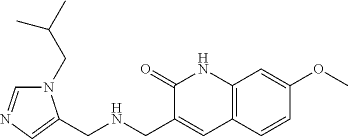 [SMILES: COc1ccc2cc(CNCc3cncn3CC(C)C)c(=O)[nH]c2c1] | 3-((((1-isobutyl-1H-imidazol-5- yl)methyl)amino)methyl)-7- methoxyquinolin-2(1H)-one |
| CPD- 105 | 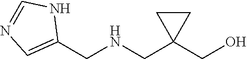 [SMILES: OCC1(CNCc2cnc[nH]2)CC1] | (1-((((1H-imidazol-5- yl)methyl)amino) methyl)cyclopropyl) methanol |

### Preparations of Compounds

The compounds used in the chemical reactions described herein may be made according to organic synthesis techniques known to those skilled in this art, starting from commercially available chemicals and/or from compounds described in the chemical literature. “Commercially available chemicals” are obtained from standard commercial sources including Acros Organics (Pittsburgh, PA), Aldrich Chemical (Milwaukee, WI, including Sigma Chemical and Fluka), Apin Chemicals Ltd. (Milton Park, UK), Avocado Research (Lancashire, U.K.), BDH Inc. (Toronto, Canada), Bionet (Cornwall, U.K.), Chemservice Inc. (West Chester, PA), Crescent Chemical Co. (Hauppauge, NY), Eastman Organic Chemicals, Eastman Kodak Company (Rochester, NY), Fisher Scientific Co. (Pittsburgh, PA), Fisons Chemicals (Leicestershire, UK), Frontier Scientific (Logan, UT), ICN Biomedicals, Inc. (Costa Mesa, CA), Key Organics (Cornwall, U.K.), Lancaster Synthesis (Windham, NH), Maybridge Chemical Co. Ltd. (Cornwall, U.K.), Parish Chemical Co. (Orem, UT), Pfaltz & Bauer, Inc. (Waterbury, CN), Polyorganix (Houston, TX), Pierce Chemical Co. (Rockford, IL), Riedel de Haen AG (Hanover, Germany), Spectrum Quality Product, Inc. (New Brunswick, NJ), TCI America (Portland, OR), Trans World Chemicals, Inc. (Rockville, MD), and Wako Chemicals USA, Inc. (Richmond, VA).

Suitable reference books and treatise that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatise that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J.C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

Alternatively, specific and analogous reactants can be identified through the indices of known chemicals and reactions prepared by the Chemical Abstract Service of the American Chemical Society, which are available in most public and university libraries, as well as through on-line databases (contact the American Chemical Society, Washington, D.C. for more details). Chemicals that are known but not commercially available in catalogs are optionally prepared by custom chemical synthesis houses, where many of the standard chemical supply houses (e.g., those listed above) provide custom synthesis services. A reference for the preparation and selection of pharmaceutical salts of the compound described herein is P. H. Stahl & C. G. Wermuth “Handbook of Pharmaceutical Salts”, Verlag Helvetica Chimica Acta, Zurich, 2002.

The compounds described herein may be prepared using the general methods in the art of organic synthesis, as described in the Examples section. Alternative synthetic methods are also used to generate the compounds described herein.

### Characterization of Exemplary Compounds

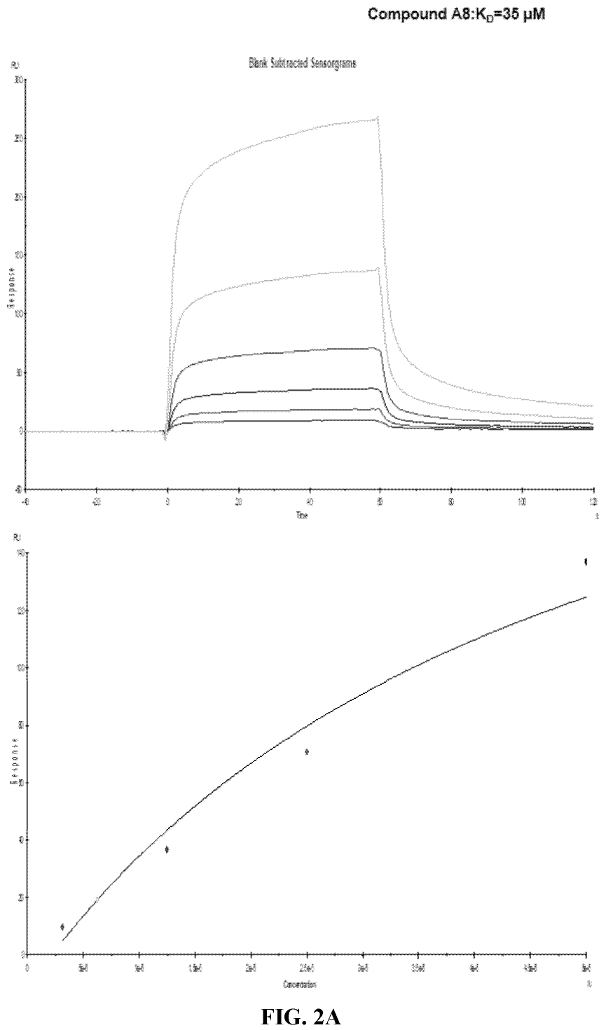

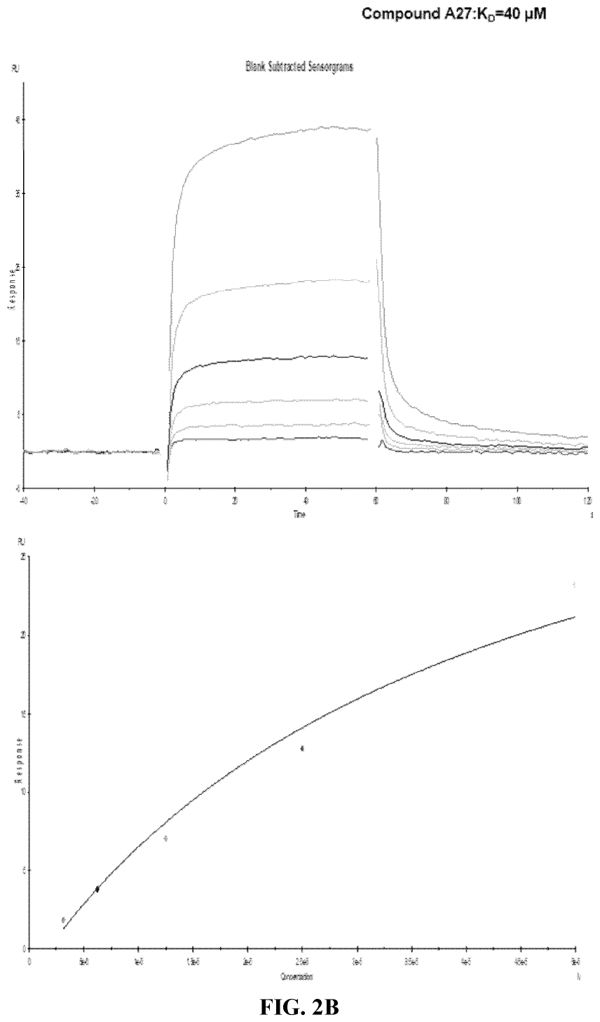

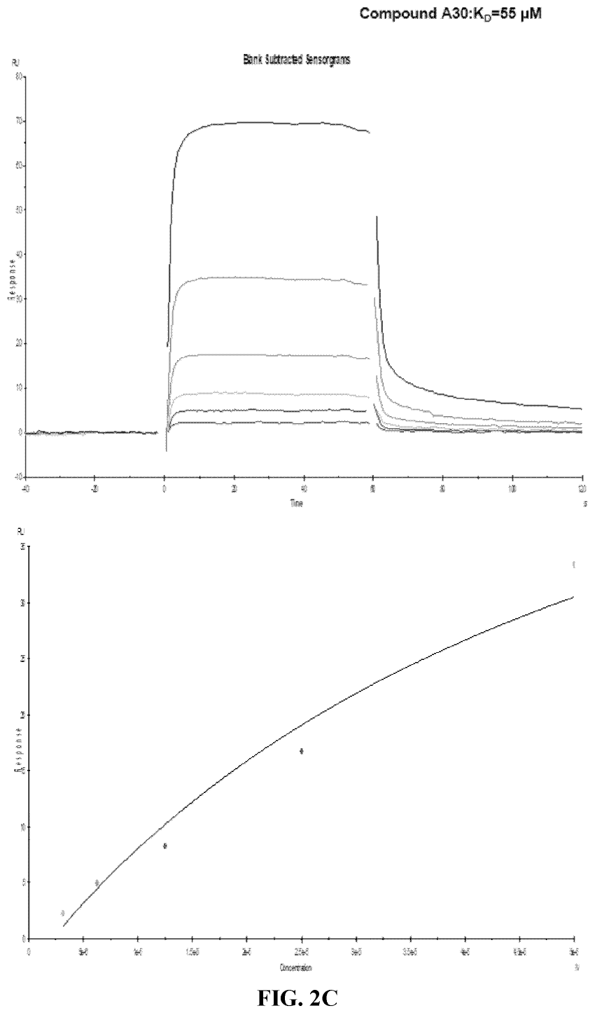

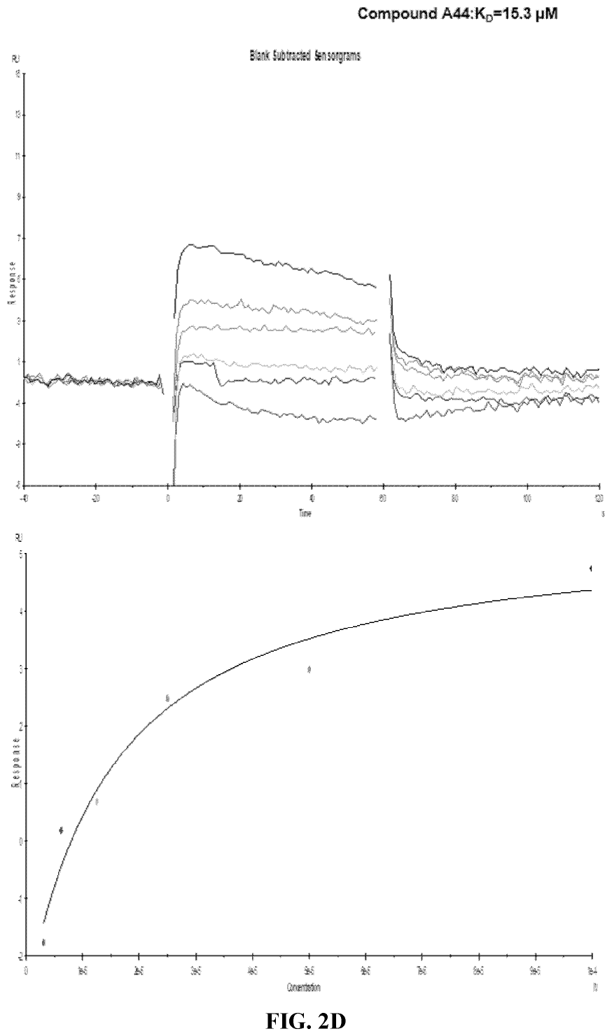

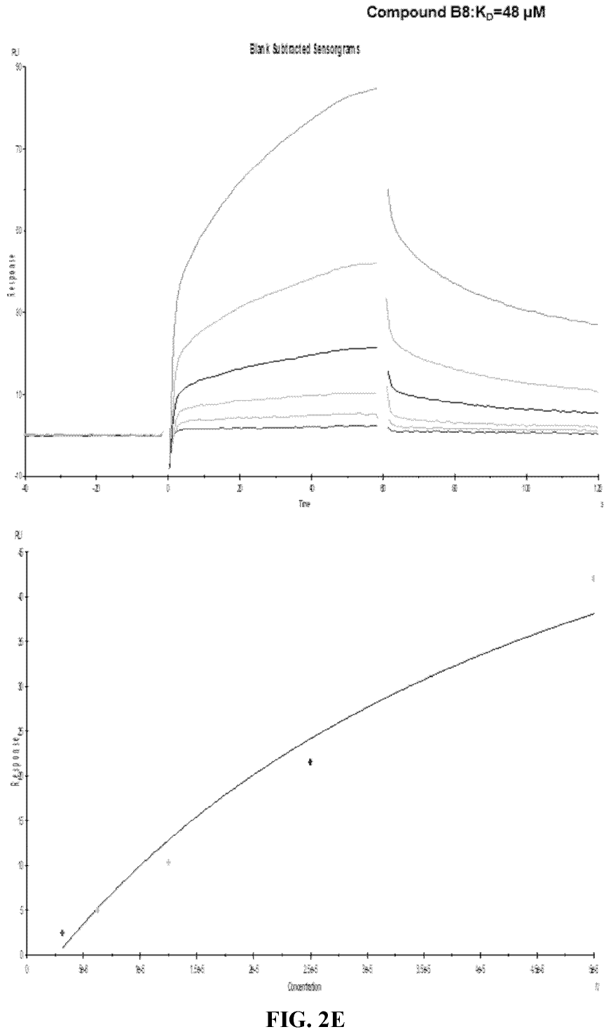

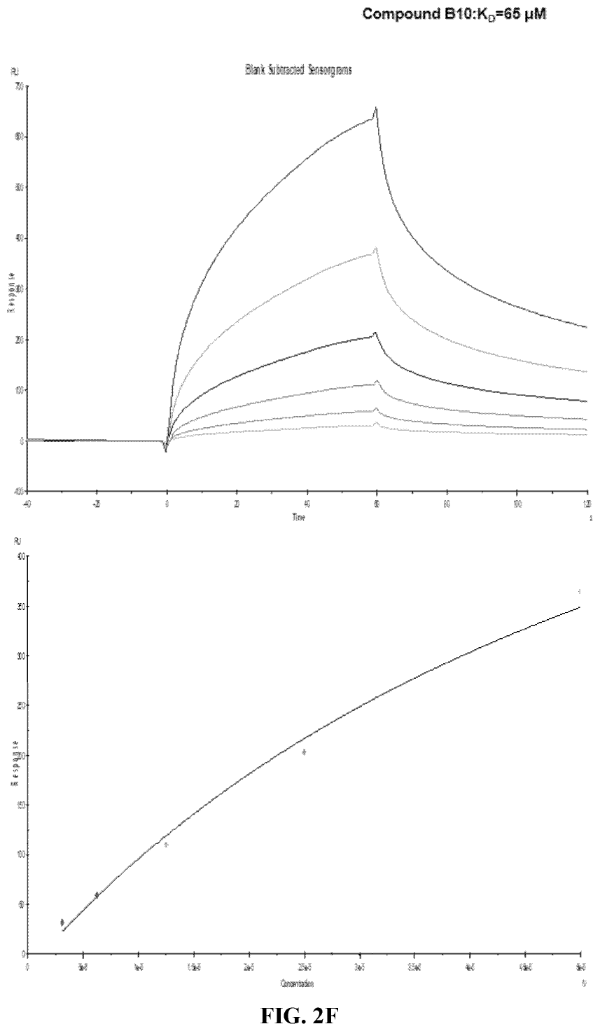

Binding affinities of specific exemplary compounds to DCAF1 (1058-1396), which is a fragment of a DCAF1 protein that includes amino acid residues A1058 to E1396 were determined by a surface plasmon resonance (SPR) assay. Briefly, purified DCAF1 (1058-1396) proteins were immobilized at a density of 9,000-11,000 resonance units (RUs) on a CMS sensor chip. Sensorgrams were recorded at different concentrations of compounds in multi-cycle kinetic format. Data were analyzed using a steady state affinity model through Biacore Evaluation Software to provide equivalent dissociation constants (K_{d}). Data showed that the exemplary compounds bound to DCAF1 in a concentration-dependent manner, and some binding affinities (K_{d}) ranged from 15 μM to 65 μM (FIG. 2A-2F, Table 2).

### Methods of Binding or Modulating DCAF1

In some embodiments, a compound described herein is used to bind a DCAF1 protein. The compound may include a compound of Table 1 or formula Ia, I, IIa, II, or III In some embodiments, a compound described herein is used to modulate a DCAF1 protein. In some embodiments, a compound described herein is used to inhibit a DCAF1 protein. Some embodiments include contacting a DCAF1 protein with a compound described herein. The contact may include administration of the compound to a subject comprising the DCAF1 protein. The contact may include administration of the compound to a cell comprising the DCAF1 protein. The contact may include administration of the compound to a sample comprising the DCAF1 protein. The contact may include administration of the compound to a solution comprising the DCAF1 protein. The contact may be in vivo. The contact may be in vitro. The compound may bind to the DCAF1 protein with a binding affinity described herein.

In some embodiments, a compound described herein binds a DCAF1 protein such as a full-length DCAF1 protein. In some embodiments, a compound described herein binds a DCAF1 fragment.

### Methods of Treatment and Pharmaceutical Compositions

In certain embodiments, a compound described herein is used to treat a subject. Some embodiments include administering a compound described herein to a subject, for example administering any compound of Table 1 or formula Ia, I, IIa, II, or III to a subject. Some embodiments include administering a compound described herein to a subject in need thereof. Some embodiments include administering a pharmaceutical composition comprising the compound to a subject. Some embodiments include providing a compound or pharmaceutical composition described herein for administration to a subject.

In some embodiments, a modified protein disclosed herein is formed in vivo upon administration of the compound or pharmaceutical composition to the subject. In some embodiments, a ligand-protein complex disclosed herein is formed by administration of the compound or pharmaceutical composition to the subject.

In certain embodiments, the compound as described herein is administered as a pure chemical. In other embodiments, the compound described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21^{st} Ed. Mack Pub. Co., Easton, PA (2005)). One embodiment provides a pharmaceutical composition comprising a compound described herein, or a pharmaceutically acceptable salt thereof, and a pharmaceutically acceptable excipient.

Provided herein is a pharmaceutical composition comprising at least one compound described herein, or a stereoisomer, pharmaceutically acceptable salt, or N-oxide thereof, together with one or more pharmaceutically acceptable carriers. The carrier(s) (or excipient(s)) is acceptable or suitable if the carrier is compatible with the other ingredients of the composition and not deleterious to the recipient (i.e., the subject or patient) of the composition. In some embodiments, the excipient comprises a buffer or solution. In some embodiments, the pharmaceutical composition is sterile.

In certain embodiments, a compound described herein is substantially pure, in that it contains less than about 5%, or less than about 1%, or less than about 0.1%, of other organic small molecules, such as unreacted intermediates or synthesis by-products that are created, for example, in one or more of the steps of a synthesis method.

Some embodiments include use of a compound described herein, use of a ligand-DCAF1 complex, or use of an in vivo modified DCAF1 protein. In some embodiments, the use comprises administration of the compound to a subject. In some embodiments, the use comprises contact of a sample with the compound.

Examples of subjects include vertebrates, animals, mammals, dogs, cats, cattle, rodents, mice, rats, primates, monkeys, and humans. In some embodiments, the subject is a mammal. In some embodiments, the subject is a human.

In some embodiments, administering the compound to a subject comprises administering an effective amount of the compound. In some embodiments, the administration is intravenous. In some embodiments, the administration comprises an injection. In some embodiments, the administration is local. In some embodiments, the administration is systemic.

In some embodiments, the sample is a biological sample. In some embodiments, the biological sample comprises a tissue, a cell, or a biological fluid. In some embodiments, the contact is in vitro. In some embodiments, the contact is in vivo.

### Definitions

As used herein and in the appended claims, the singular forms “a,” “and,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range, in some instances, will vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

As used in the specification and appended claims, unless specified to the contrary, the following terms have the meaning indicated below.
“Amino” refers to the —NH₂ radical.
“Cyano” refers to the —CN radical.
“Nitro” refers to the —NO₂ radical.
“Oxa” refers to the —O— radical.
“Oxo” refers to the ═O radical.
“Thioxo” refers to the ═S radical.
“Imino” refers to the ═N—H radical.
“Oximo” refers to the ═N—OH radical.
“Hydrazino” refers to the ⊚N—NH₂ radical.

“Alkyl” refers to a straight or branched hydrocarbon chain radical consisting solely of carbon and hydrogen atoms, containing no unsaturation, having from one to fifteen carbon atoms (e.g., C₁-C₁₅ alkyl). In certain embodiments, an alkyl comprises one to thirteen carbon atoms (e.g., C₁-C₁₃ alkyl). In certain embodiments, an alkyl comprises one to eight carbon atoms (e.g., C₁-C₈ alkyl). In other embodiments, an alkyl comprises one to five carbon atoms (e.g., C₁-C₅ alkyl). In other embodiments, an alkyl comprises one to four carbon atoms (e.g., C₁-C₄ alkyl). In other embodiments, an alkyl comprises one to three carbon atoms (e.g., C₁-C₃ alkyl). In other embodiments, an alkyl comprises one to two carbon atoms (e.g., C₁-C₂ alkyl). In other embodiments, an alkyl comprises one carbon atom (e.g., C₁ alkyl). In other embodiments, an alkyl comprises five to fifteen carbon atoms (e.g., C₅-C₁₅ alkyl). In other embodiments, an alkyl comprises five to eight carbon atoms (e.g., C₅-C₈ alkyl). In other embodiments, an alkyl comprises two to five carbon atoms (e.g., C₂-C₅ alkyl). In other embodiments, an alkyl comprises three to five carbon atoms (e.g., C₃-C₅ alkyl). In other embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond. Unless stated otherwise specifically in the specification, an alkyl group is optionally substituted by one or more of the following substituents: halo, cyano, nitro, oxo, thioxo, imino, oximo, trimethylsilanyl, R^{a}, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkoxy” refers to a radical bonded through an oxygen atom of the formula —0-alkyl, where alkyl is an alkyl chain as defined above.

“Haloalkyl” refers to an alkyl group that is substituted by one or more halogens. Exemplary haloalkyl groups include trifluoromethyl, difluoromethyl, trichloromethyl, 2,2,2-trifluoroethyl, 1,2-difluoroethyl, 3-bromo-2-fluoropropyl, and 1,2-dibromoethyl.

“Heteroalkyl”, “heteroalkenyl” and “heteroalkynyl” refer to substituted or unsubstituted alkyl, alkenyl and alkynyl groups which respectively have one or more skeletal chain atoms selected from an atom other than carbon. Exemplary skeletal chain atoms selected from an atom other than carbon include, e.g., O, N, P, Si, S, or combinations thereof, wherein the nitrogen, phosphorus, and sulfur atoms may optionally be oxidized and the nitrogen heteroatom may optionally be quaternized. If given, a numerical range refers to the chain length in total. For example, a 3- to 8-membered heteroalkyl has a chain length of 3 to 8 atoms. Connection to the rest of the molecule may be through either a heteroatom or a carbon in the heteroalkyl, heteroalkenyl or heteroalkynyl chain. Unless stated otherwise specifically in the specification, a heteroalkyl, heteroalkenyl, or heteroalkynyl group is optionally substituted by one or more substituents such as those substituents described herein.

“Alkenyl” refers to a straight or branched hydrocarbon chain radical group consisting solely of carbon and hydrogen atoms, containing at least one carbon-carbon double bond, and having from two to twelve carbon atoms. In certain embodiments, an alkenyl comprises two to eight carbon atoms. In other embodiments, an alkenyl comprises two to four carbon atoms. The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like. Unless stated otherwise specifically in the specification, an alkenyl group is optionally substituted by one or more of the following substituents: halo, cyano, nitro, oxo, thioxo, imino, oximo, trimethylsilanyl, R^{a}, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkynyl” refers to a straight or branched hydrocarbon chain radical group consisting solely of carbon and hydrogen atoms, containing at least one carbon-carbon triple bond, having from two to twelve carbon atoms. In certain embodiments, an alkynyl comprises two to eight carbon atoms. In other embodiments, an alkynyl comprises two to six carbon atoms. In other embodiments, an alkynyl comprises two to four carbon atoms. The alkynyl is attached to the rest of the molecule by a single bond, for example, ethynyl, propynyl, butynyl, pentynyl, hexynyl, and the like. Unless stated otherwise specifically in the specification, an alkynyl group is optionally substituted by one or more of the following substituents: halo, cyano, nitro, oxo, thioxo, imino, oximo, trimethylsilanyl, R^{a}, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜR^{a} (where t is 1 or 2), —S(O)tRa (where t is 1 or 2) and —S(O)tN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkylene” or “alkylene chain” refers to a straight or branched divalent hydrocarbon chain linking the rest of the molecule to a radical group, consisting solely of carbon and hydrogen, containing no unsaturation and having from one to twelve carbon atoms, for example, methylene, ethylene, propylene, n-butylene, and the like. The alkylene chain is attached to the rest of the molecule through a single bond and to the radical group through a single bond. The points of attachment of the alkylene chain to the rest of the molecule and to the radical group are through one carbon in the alkylene chain or through any two carbons within the chain. In certain embodiments, an alkylene comprises one to eight carbon atoms (e.g., C₁-C₈ alkylene). In other embodiments, an alkylene comprises one to five carbon atoms (e.g., C₁-C₅ alkylene). In other embodiments, an alkylene comprises one to four carbon atoms (e.g., C₁-C₄ alkylene). In other embodiments, an alkylene comprises one to three carbon atoms (e.g., C₁-C₃ alkylene). In other embodiments, an alkylene comprises one to two carbon atoms (e.g., C₁-C₂ alkylene). In other embodiments, an alkylene comprises one carbon atom (e.g., C₁ alkylene). In other embodiments, an alkylene comprises five to eight carbon atoms (e.g., C₅-C₈ alkylene). In other embodiments, an alkylene comprises two to five carbon atoms (e.g., C₂-C₅ alkylene). In other embodiments, an alkylene comprises three to five carbon atoms (e.g., C₃-C₅ alkylene). Unless stated otherwise specifically in the specification, an alkylene chain is optionally substituted by one or more of the following substituents: halo, cyano, nitro, oxo, thioxo, imino, oximo, trimethylsilanyl, R^{a}, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from five to eighteen carbon atoms, where at least one of the rings in the ring system is fully unsaturated, i.e., it contains a cyclic, delocalized (4n+2) π-electron system in accordance with the Hiickel theory. The ring system from which aryl groups are derived include, but are not limited to, groups such as benzene, fluorene, indane, indene, tetralin and naphthalene. Unless stated otherwise specifically in the specification, the term “aryl” or the prefix “ar-” (such as in “aralkyl”) is meant to include aryl radicals optionally substituted by one or more substituents independently selected from alkyl, alkenyl, alkynyl, halo, fluoroalkyl, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, R^{a}, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R b is independently a direct bond or a straight or branched alkylene or alkenylene chain, and Rc is a straight or branched alkylene or alkenylene chain, and where each of the above substituents is unsubstituted unless otherwise indicated.

“Aralkyl” refers to a radical of the formula —R^{c}-aryl where IR^{c} is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Carbocyclyl” refers to a stable non-aromatic monocyclic or polycyclic hydrocarbon radical consisting solely of carbon and hydrogen atoms, which includes fused or bridged ring systems, having from three to fifteen carbon atoms. In certain embodiments, a carbocyclyl comprises three to ten carbon atoms. In other embodiments, a carbocyclyl comprises five to seven carbon atoms. The carbocyclyl is attached to the rest of the molecule by a single bond. Carbocyclyl is saturated (i.e., containing single C—C bonds only) or unsaturated (i.e., containing one or more double bonds or triple bonds). A fully saturated carbocyclyl radical is also referred to as “carbocyclyl.” Examples of monocyclic carbocyclyls include, e.g., cyclopropyl, cyclobutyl, cyclopentyl, cyclohexyl, cycloheptyl, and cyclooctyl. An unsaturated carbocyclyl is also referred to as “cycloalkenyl.” Examples of monocyclic cycloalkenyls include, e.g., cyclopentenyl, cyclohexenyl, cycloheptenyl, and cyclooctenyl. Polycyclic carbocyclyl radicals include, for example, adamantyl, norbornyl (i.e., bicyclo[2.2.1]heptanyl), norbornenyl, decalinyl, 7,7-dimethyl-bicyclo[2.2.1]heptanyl, and the like. Unless otherwise stated specifically in the specification, the term “carbocyclyl” is meant to include carbocyclyl radicals that are optionally substituted by one or more substituents independently selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, R^{a}, —R^{b}—OR^{a}, —R^{b}—OC(O)—R^{a}, —R^{b}OC(O)—OR^{a}, —R^{b}OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R b is independently a direct bond or a straight or branched alkylene or alkenylene chain, and Rc is a straight or branched alkylene or alkenylene chain, and where each of the above substituents is unsubstituted unless otherwise indicated.

“Carbocyclylalkyl” refers to a radical of the formula —R° -carbocyclyl where Rc is an alkylene chain as defined above. The alkylene chain and the carbocyclyl radical are optionally substituted as defined above.

“Halo” or “halogen” refers to bromo, chloro, fluoro or iodo substituents.

“Fluoroalkyl” refers to an alkyl radical, as defined above, that is substituted by one or more fluoro radicals, as defined above, for example, trifluoromethyl, difluoromethyl, fluoromethyl, 2,2,2-trifluoroethyl, 1-fluoromethyl-2-fluoroethyl, and the like. In some embodiments, the alkyl part of the fluoroalkyl radical is optionally substituted as defined above for an alkyl group.

“Heterocyclyl” refers to a stable 3- to 18-membered non-aromatic ring radical that comprises two to twelve carbon atoms and from one to six heteroatoms selected from nitrogen, oxygen and sulfur. Unless stated otherwise specifically in the specification, the heterocyclyl radical is a monocyclic, bicyclic, tricyclic or tetracyclic ring system, which optionally includes fused or bridged ring systems. The heteroatoms in the heterocyclyl radical are optionally oxidized. One or more nitrogen atoms, if present, are optionally quaternized. The heterocyclyl radical is partially or fully saturated. The heterocyclyl is attached to the rest of the molecule through any atom of the ring(s). Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, the term “heterocyclyl” is meant to include heterocyclyl radicals as defined above that are optionally substituted by one or more substituents selected from alkyl, alkenyl, alkynyl, halo, fluoroalkyl, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, R^{a}, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R b is independently a direct bond or a straight or branched alkylene or alkenylene chain, and RC is a straight or branched alkylene or alkenylene chain, and where each of the above substituents is unsubstituted unless otherwise indicated.

“N-heterocyclyl” or “N-attached heterocyclyl” refers to a heterocyclyl radical as defined above containing at least one nitrogen and where the point of attachment of the heterocyclyl radical to the rest of the molecule is through a nitrogen atom in the heterocyclyl radical. An N-heterocyclyl radical is optionally substituted as described above for heterocyclyl radicals. Examples of such N-heterocyclyl radicals include, but are not limited to, 1-morpholinyl, 1-piperidinyl, 1-piperazinyl, 1-pyrrolidinyl, pyrazolidinyl, imidazolinyl, and imidazolidinyl.

“C-heterocyclyl” or “C-attached heterocyclyl” refers to a heterocyclyl radical as defined above containing at least one heteroatom and where the point of attachment of the heterocyclyl radical to the rest of the molecule is through a carbon atom in the heterocyclyl radical. A C-heterocyclyl radical is optionally substituted as described above for heterocyclyl radicals. Examples of such C-heterocyclyl radicals include, but are not limited to, 2-morpholinyl, 2- or 3- or 4-piperidinyl, 2-piperazinyl, 2- or 3-pyrrolidinyl, and the like.

“Heteroaryl” refers to a radical derived from a 3- to 18-membered aromatic ring radical that comprises two to seventeen carbon atoms and from one to six heteroatoms selected from nitrogen, oxygen and sulfur. As used herein, the heteroaryl radical is a monocyclic, bicyclic, tricyclic or tetracyclic ring system, wherein at least one of the rings in the ring system is fully unsaturated, i.e., it contains a cyclic, delocalized (4n+2) π-electron system in accordance with the Hückel theory. Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo [h] quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pridinyl, and thiophenyl (i.e. thienyl). Unless stated otherwise specifically in the specification, the term “heteroaryl” is meant to include heteroaryl radicals as defined above which are optionally substituted by one or more substituents selected from alkyl, alkenyl, alkynyl, halo, fluoroalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, R^{a}, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“N-heteroaryl” refers to a heteroaryl radical as defined above containing at least one nitrogen and where the point of attachment of the heteroaryl radical to the rest of the molecule is through a nitrogen atom in the heteroaryl radical. An N-heteroaryl radical is optionally substituted as described above for heteroaryl radicals.

“C-heteroaryl” refers to a heteroaryl radical as defined above and where the point of attachment of the heteroaryl radical to the rest of the molecule is through a carbon atom in the heteroaryl radical. A C-heteroaryl radical is optionally substituted as described above for heteroaryl radicals.

The compounds disclosed herein, in some embodiments, contain one or more asymmetric centers and thus give rise to enantiomers, diastereomers, and other stereoisomeric forms that are defined, in terms of absolute stereochemistry, as (R)- or (S)-. Unless stated otherwise, it is intended that all stereoisomeric forms of the compounds disclosed herein are contemplated by this disclosure. When the compounds described herein contain alkene double bonds, and unless specified otherwise, it is intended that this disclosure includes both E and Z geometric isomers (e.g., cis or trans.) Likewise, all possible isomers, as well as their racemic and optically pure forms, and all tautomeric forms are also intended to be included. The term “geometric isomer” refers to E or Z geometric isomers (e.g., cis or trans) of an alkene double bond. The term “positional isomer” refers to structural isomers around a central ring, such as ortho-, meta-, and para- isomers around a benzene ring.

A “tautomer” refers to a molecule wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

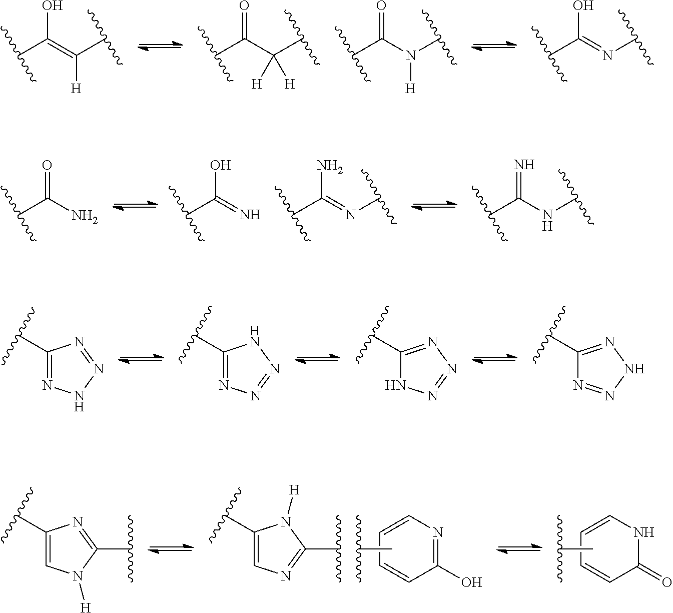

The compounds disclosed herein, in some embodiments, are used in different enriched isotopic forms, e.g., enriched in the content of ²H, ³H, ¹¹C, ¹³C and/or ¹⁴C. In one particular embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

Unless otherwise stated, structures depicted herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of the present disclosure optionally contain unnatural proportions of atomic isotopes at one or more atoms that constitute such compounds. For example, the compounds may be labeled with isotopes, such as for example, deuterium (²H), tritium (³H), iodine-125 (¹²⁵I) or carbon-14 (¹⁴C). Isotopic substitution with ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, ¹²⁵I are all contemplated. All isotopic variations of the compounds of the present invention, whether radioactive or not, are encompassed within the scope of the present invention.

In certain embodiments, the compounds disclosed herein have some or all of the ¹H atoms replaced with ²H atoms. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)] 2000, 110 pp; George W.; Varma, Rajender S. The Synthesis of Radiolabeled Compounds via Organometallic Intermediates, Tetrahedron, 1989, 45 (21), 6601-21; and Evans, E. Anthony. Synthesis of radiolabeled compounds, J. Radioanal. Chem., 1981, 64 (1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

“Pharmaceutically acceptable salt” includes both acid and base addition salts. A pharmaceutically acceptable salt of any one of the compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997)). Acid addition salts of basic compounds are, in some embodiments, prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts are, in some embodiments, formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

### EXAMPLES

The following examples are set forth to illustrate more clearly the principle and practice of instances disclosed herein to those skilled in the art and are not to be construed as limiting the scope of any claimed instances. Unless otherwise stated, all parts and percentages are on a weight basis.

Non-limiting examples of compound synthesis schemes are provided below.

### Example 1: 1-(1-Methyl-1H-pyrrol-2-yl)-N-(pyridin-4-ylmethyl)methanamine (CPD-001)

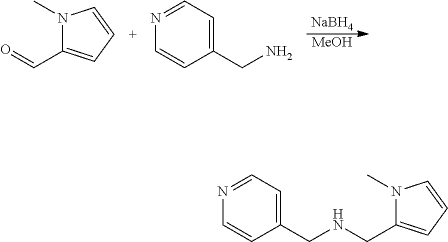

A mixture of 1-methyl-1H-pyrrole-2-carbaldehyde (218 mg, 2.0 mmol) and pyridin-4-ylmethanamine (216 mg, 2.0 mmol) in MeOH (5 mL) was stirred at rt for 16 h. Then NaBH₄ (91 mg, 2.4 mmol) was added and stirred at rt for 30 min. After the mixture was concentrated, the residue was diluted with DCM, filtered, and concentrated. The residue was purified by silica gel chromatography (DCM:MeOH=100:1 to 10:1) to afford the title compound (239 mg, yield: 59%) as a colorless oil. MS (ESI) m/z=201.9 [M+H]⁺. ¹H NMR (400 MHz, DMSO-d₆) δ 8.54 (d, J=6.0 Hz, 2H), 7.27 (d, J=5.6 Hz, 2H), 6.60 (t, J=2.0 Hz, 1H), 6.05-6.02 (m, 2H), 3.83 (s, 2H), 3.74 (s, 2H), 3.65 (s, 3H).

### Example 2: 1-(1-Methyl-1H-pyrrol-2-yl)-N-(pyridin-3-ylmethyl)methanamine (CPD-002)

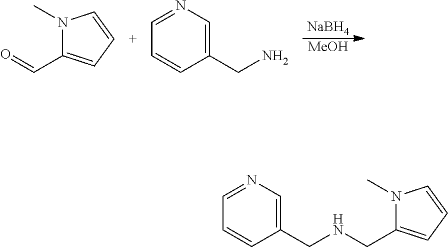

CPD-002 was synthesized following the standard procedure for preparing CPD-001 (235 mg, yield: 58%) as a colorless oil. MS (ESI) m/z=202.5 [M+H]⁺. ¹H NMR (400 MHz, DMSO-d₆) δ 8.58 (d, J=1.6 Hz, 1H), 8.50 (dd, J=1.6, 4.4 Hz, 1H), 7.67 (d, J=7.6 Hz, 1H), 7.27-7.24 (m, 1H), 6.59 (t, J=2.4 Hz, 1H), 6.05-6.02 (m, 2H), 3.83 (s, 2H), 3.74 (s, 2H), 3.63 (s, 3H).

### Example 3: 1-(1-Methyl-1H-pyrrol-2-yl)-N-(pyridin-2-ylmethyl)methanamine (CPD-003)

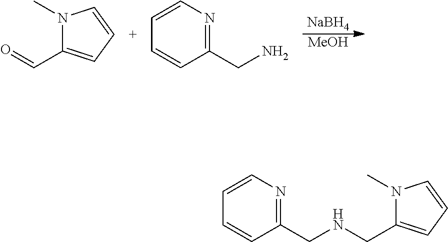

CPD-003 was synthesized following the standard procedure for preparing CPD-001 (263 mg, yield: 65%) as a colorless oil. MS (ESI) m/z=202.5 [M+H]⁺. 1H NMR (400 MHz, DMSO-d6) δ 8.55 (d, J=4.4 Hz, 1H), 7.66-7.62 (m, 1H), 7.31 (d, J=8.4 Hz, 1H), 7.16 (dd, J=1.6, 7.2 Hz, 1H), 6.58 (t, J=2.0 Hz, 1H), 6.04 (d, J=2.0 Hz, 2H), 3.93 (s, 2H), 3.78 (s, 2H), 3.65 (s, 3H).

### Example 4: 1-(6-Aminohexyl)-N-(pyridin-4-ylmethyl)-1H-pyrrole-2-carboxamide (CPD-004)

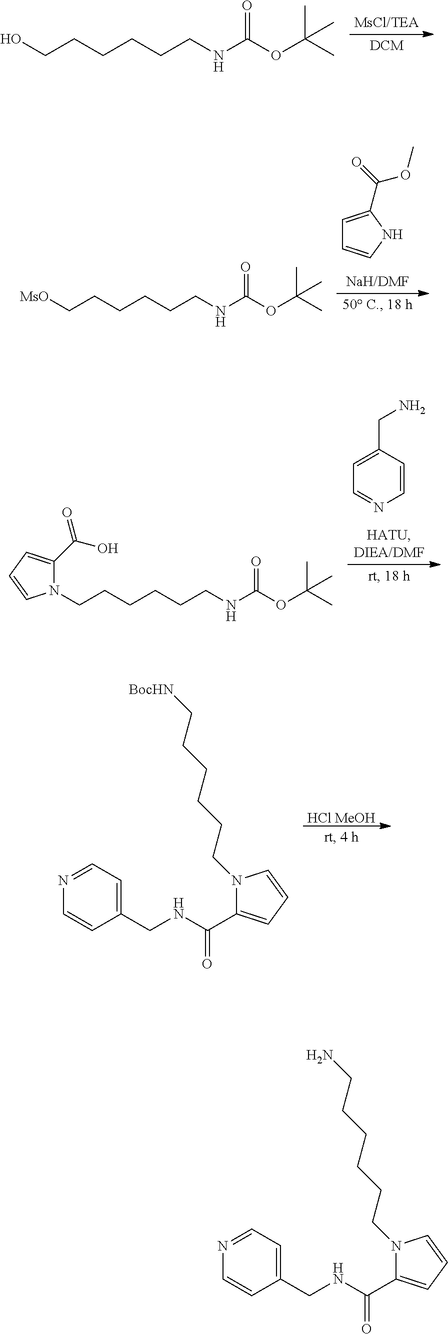

Step 1. Synthesis of 6-((tert-butoxycarbonyl)amino)hexyl methanesulfonate

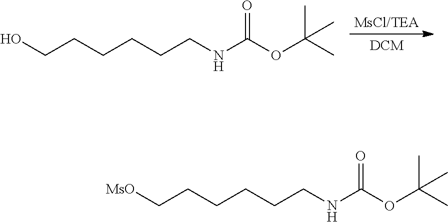 [SMILES: CC(C)(C)OC(=O)NCCCCCCO ; CC(C)(C)OC(=O)NCCCCCCOS(C)(=O)=O]

To a mixture of tert-butyl (6-hydroxyhexyl)carbamate (3.2 g, 14.7 mmol) and TEA (1.78 g, 17.64 mmol) in DCM (32 ml) was added MsCl (1.76 g, 15.4 mmol) in an ice bath. After the mixture was stirred at rt for 5 h, the mixture was diluted with water (50 mL) and extracted with DCM (3×30 mL). The combined organic layers were washed with brine, concentrated, and purified by silica gel chromatography (petroleum ether:EtOAc=8:1) to afford the title compound (2.8 g, yield: 64%) as a yellow solid.

Step 2. Synthesis of 1-(6-((tert-butoxycarbonyl)amino)hexyl)-1H-pyrrole-2-carboxylic acid

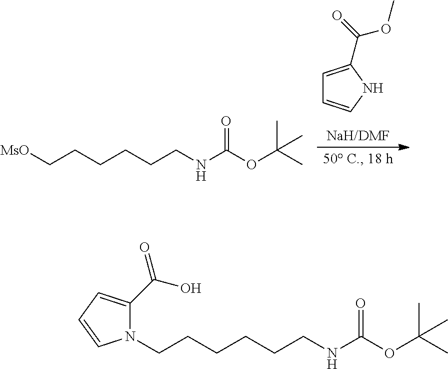

To a mixture of methyl 1H-pyrrole-2-carboxylate (250 mg, 2 mmol) in THF (4 mL) was added NaH (60%, 160 mg, 4 mmol) at 10° C. The reaction mixture was stirred for 30 min, before 6-((tert-butoxycarbonyl)amino)hexyl methanesulfonate (592 mg, 2 mmol) was added at rt. After the mixture was stirred at 50° C. for 18 h, the reaction was quenched with water (8 mL). The aqueous phase was extracted with EtOAc (3×8 mL). The combined organic layers were washed with brine, concentrated, and purified by silica gel chromatography (petroleum ether:EtOAc=1:2) to afford the title compound (220 mg, yield: 84%) as white solid.

Step 3. Synthesis of tert-butyl (6-(2-((pyridin-4-ylmethyl)carbamoyl)-1H-pyrrol-1-yl)hexyl)carbamate

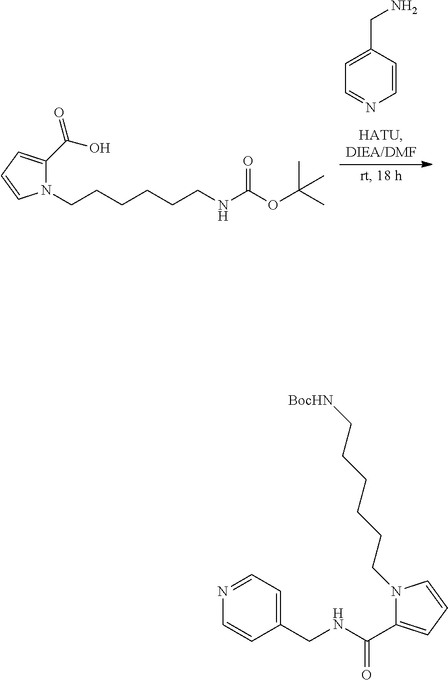 [SMILES: NCc1ccncc1 ; CC(C)(C)OC(=O)NCCCCCCn1cccc1C(=O)O ; CC(C)(C)OC(=O)NCCCCCCn1cccc1C(=O)NCc1ccncc1]

A mixture of pyridin-4-ylmethanamine (80.6 mg, 0.746 mmol), 1-(6-((tert-butoxycarbonyl)amino)hexyl)-1H-pyrrole-2-carboxylic acid (220 mg, 0.71 mmol), HATU (296.4 mg, 0.78 mmol) and DIEA (274.8 mg, 2.14 mmol) in DMF (4 mL) was stirred at rt for 18 h. After the reaction was quenched with water (10 mL), the aqueous phase was extracted with EtOAc (3×10 mL). The combined organic layers were washed with brine, concentrated, and purified by silica gel chromatography (petroleum ether:EtOAc=2:1) to afford the title compound (70 mg, yield: 67%) as white solid. MS (ESI) m/z=401.1 [M+H]⁺.

Step 4. Synthesis of 1-(6-aminohexyl)-N-(pyridin-4-ylmethyl)-1H-pyrrole-2-carboxamide

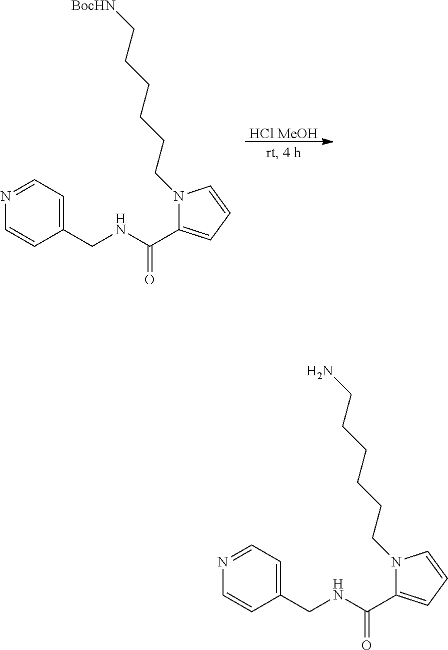 [SMILES: CC(C)(C)OC(=O)NCCCCCCn1cccc1C(=O)NCc1ccncc1 ; NCCCCCCn1cccc1C(=O)NCc1ccncc1 ; "HCl MeOH"]

To a solution of tert-butyl (6-(2-((pyridin-4-ylmethyl)carbamoyl)-1H-pyrrol-1-yl)hexyl)carbamate (70 mg, 0.175 mmol) in MeOH (2 mL) was added HCl (3M in MeOH, 1 mL). The mixture was stirred at rt for 4 h, before the reaction mixture was concentrated, and neutralized with saturated NaHCO₃ aqueous solution. The aqueous phase was extracted with EtOAc (3×10 mL). The combined organic layers were washed with brine, concentrated, and purified by prep-HPLC (0.1% NH₄HCO₃ in H₂O) to afford the title compound (48 mg, yield: 91%) as clear oil. MS (ESI) m/z=301.2 [M+H]⁺. ¹H NMR (400 MHz, CDCl₃) δ 8.55 (d, J=5.6 Hz, 2H), 7.24 (d, J=6.0 Hz, 2H), 6.81 (t, J=2.0 Hz, 1H), 6.61 (dd, J=2.0, 4.0 Hz, 1H), 6.40 (t, J=5.6 Hz, 1H), 6.11 (dd, J=2.0, 6.0 Hz, 1H), 4.58 (d, J=6.0 Hz, 2H), 4.34 (t, J=7.2 Hz, 2H), 2.66 (t, J=6.8 Hz, 2H), 1.81-1.77 (m, 2H), 1.44-1.39 (m, 2H), 1.32-1.30 (m, 4H).

### Example 5: 6-(2-(((Pyridin-4-ylmethyl)amino)methyl)-1H-pyrrol-1-yl)hexan-1-amine (CPD-005)

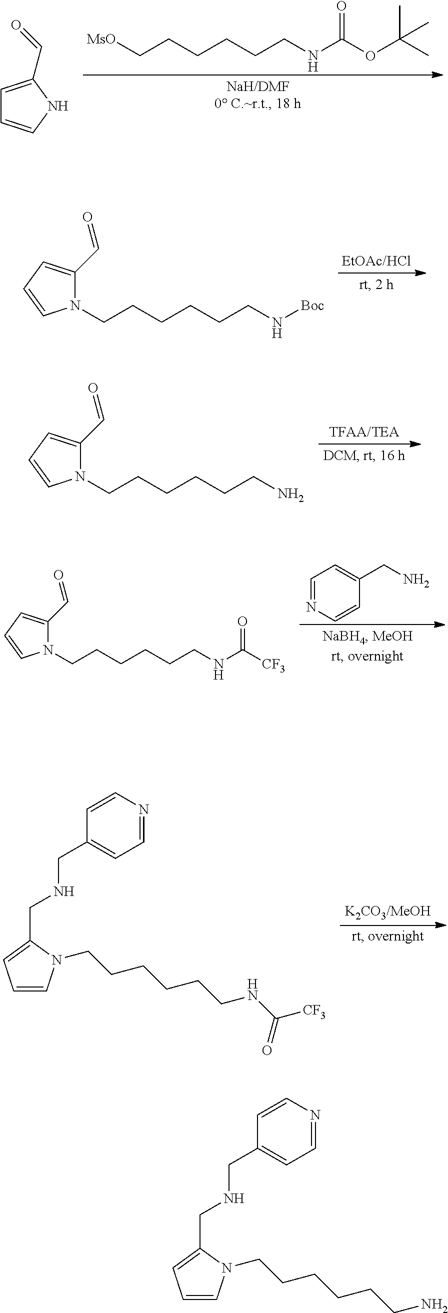

Step 1. Synthesis of tert-butyl (6-(2-formyl-1H-pyrrol-1-yl)hexyl)carbamate

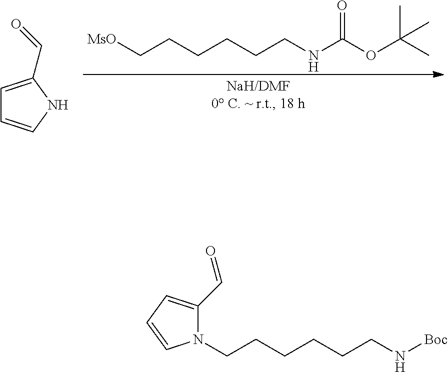 [SMILES: O=Cc1ccc[nH]1 ; CC(C)(C)OC(=O)NCCCCCCOS(C)(=O)=O ; CC(C)(C)OC(=O)NCCCCCCn1cccc1C=O]

To a solution of 1H-pyrrole-2-carbaldehyde (300 mg, 3.15 mmol) in DMF (6 mL) was added 60% NaH (252 mg, 6.30 mmol) in several portions in an ice bath. The mixture was stirred for 0.5 h, before 6-((tert-butoxycarbonyl)amino)hexyl methanesulfonate (1024 mg. 3.47 mmol) was added. After the reaction mixture was stirred at rt overnight, the reaction mixture was poured into water (30 mL), extracted with EtOAc (3×30 mL). The combined organics were washed with brine and concentrated. The residue was purified by silica gel chromatography (petroleum ether:EtOAc=50:1 to 15:1) to afford the title compound (650 mg, yield: 70%) as a colorless oil. MS (ESI) m/z=295.2 [M+H]⁺.

Step 2. Synthesis of 1-(6-aminohexyl)-1H-pyrrole-2-carbaldehyde

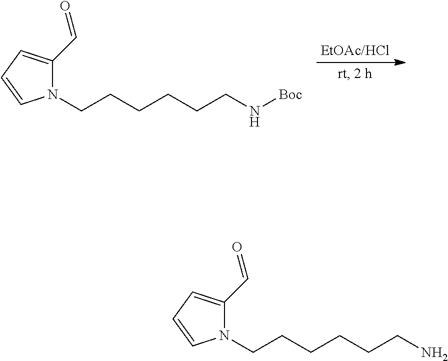 [SMILES: CC(C)(C)OC(=O)NCCCCCCn1cccc1C=O ; NCCCCCCn1cccc1C=O]

A solution of tert-butyl (6-(2-formyl-1H-pyrrol-1-yl)hexyl)carbamate (250 mg, 0.85 mmol) in HCl (3 M in EtOAc, 5 mL) was stirred at rt for 3 h. After concentration, DCM (10 mL) was added. After cooling to 0° C. with an ice bath, TEA (268 mg, 1.28 mmol) was added. The mixture was stirred at rt for 2 h. The mixture was concentrated to afford the title compound (240 mg, crude) as brown oil which was used directly in the next step without further purification. MS (ESI) m/z=195.2 [M+H]+.

Step 3. Synthesis of 2,2,2-trifluoro-N-(6-(2-formyl-1H-pyrrol-1-yl)hexyl)acetamide

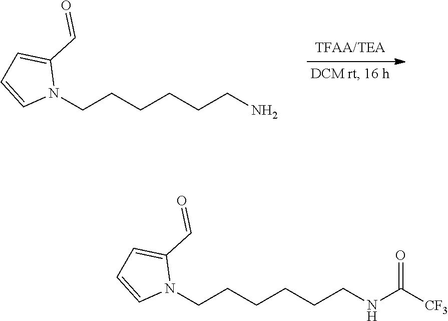 [SMILES: NCCCCCCn1cccc1C=O ; O=Cc1cccn1CCCCCCNC(=O)C(F)(F)F]

To a solution of 1-(6-aminohexyl)-1H-pyrrole-2-carbaldehyde (240 mg, 0.85 mmol) in DCM (10 mL) in an ice bath was added TEA (429 mg, 4.25 mmol) and TFAA (268 mg, 1.28 mmol). After the reaction mixture was stirred at rt for 16 h, the mixture was poured into water (10 mL). The aqueous phase was extracted with DCM (2×15 mL). The combined organic layers were washed with brine and concentrated. The residue was purified by prep-TLC (petroleum ether:EtOAc=5:1) to afford the title compound (46 mg, yield over 2 steps: 18%) as a colorless oil. MS (ESI) m/z=289.1 [M−H]⁻.

Step 4. Synthesis of 2,2,2-trifluoro-N-(6-(2-(((pyridin-4-ylmethyl)amino)methyl)-1H-pyrrol-1-yl)hexyl)acetamide

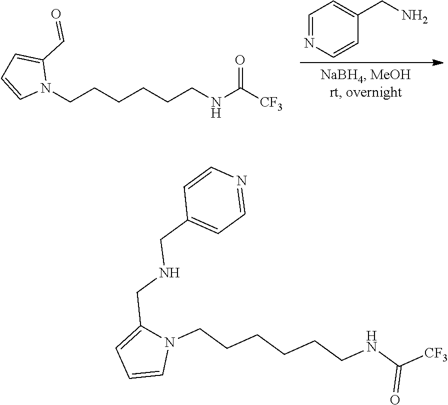 [SMILES: O=Cc1cccn1CCCCCCNC(=O)C(F)(F)F ; NCc1ccncc1 ; O=C(NCCCCCCn1cccc1CNCc1ccncc1)C(F)(F)F]

A mixture of 2,2,2-trifluoro-N-(6-(2-formyl-1H-pyrrol-1-yl)hexyl)acetamide (80 mg, 0.28 mmol) and pyridin-4-ylmethanamine (33 mg, 0.30 mmol) in MeOH (3 mL) was stirred at rt for 16 h. After cooling to 0° C. in an ice bath, NaBH₄ (10.5 mg, 0.28 mmol) was added in several portions. The reaction mixture was stirred for another 1 h, before water (10 mL) was added. The mixture was extracted with EtOAc (2×30 mL). The combined organic layers were washed with brine and concentrated. The residue was purified by prep-TLC to afford the title compound (50 mg, yield: 48%) as a colorless oil. MS (ESI) m/z=381.1 [M−H]⁻.

Step 5. Synthesis of 6-(2-(((pyridin-4-ylmethyl)amino)methyl)-1H-pyrrol-1-yl)hexan-1-amine

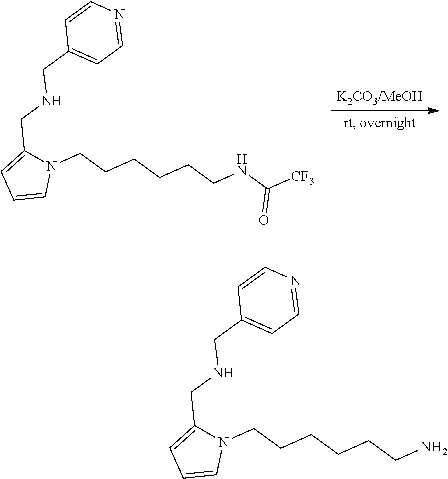 [SMILES: O=C(NCCCCCCn1cccc1CNCc1ccncc1)C(F)(F)F ; NCCCCCCn1cccc1CNCc1ccncc1]

To a solution of 2,2,2-trifluoro-N-(6-(2-(((pyridin-4-ylmethyl)amino)methyl)-1H-pyrrol-1-yl)hexyl)acetamide (45 mg, 0.12 mmol) in MeOH (2 mL) was added K₂CO₃ (58 mg, 0.42 mmol) at rt. The reaction mixture was stirred at rt for 16 h, before the mixture was filtered. The filtrate was concentrated, and the resulting residue was purified by prep-HPLC (0.1% NH₄HCO₃ in H₂O) to afford the title compound (26 mg, yield: 77%) as light-yellow oil. MS (ESI) m/z=287.2 [M+H]⁺. ¹H NMR (400 MHz, DMSO-d₆) δ 8.48 (d, J=6.0 Hz, 2H), 7.33 (d, J=5.6 Hz, 2H), 6.66 (t, J=2.0 Hz, 1H), 5.88-5.86 (m, 2H), 3.86 (t, J=7.2 Hz, 2H), 3.69 (s, 2H), 3.59 (s, 2H), 2.50-2.47 (m, 2H), 1.64-1.59 (m, 2H), 1.30-1.23 (m, 6H).

### Example 6: 6-(2-(((Pyridin-4-ylmethyl)amino)methyl)-1H-pyrrol-1-yl)hexan-1-amine (CPD-006)

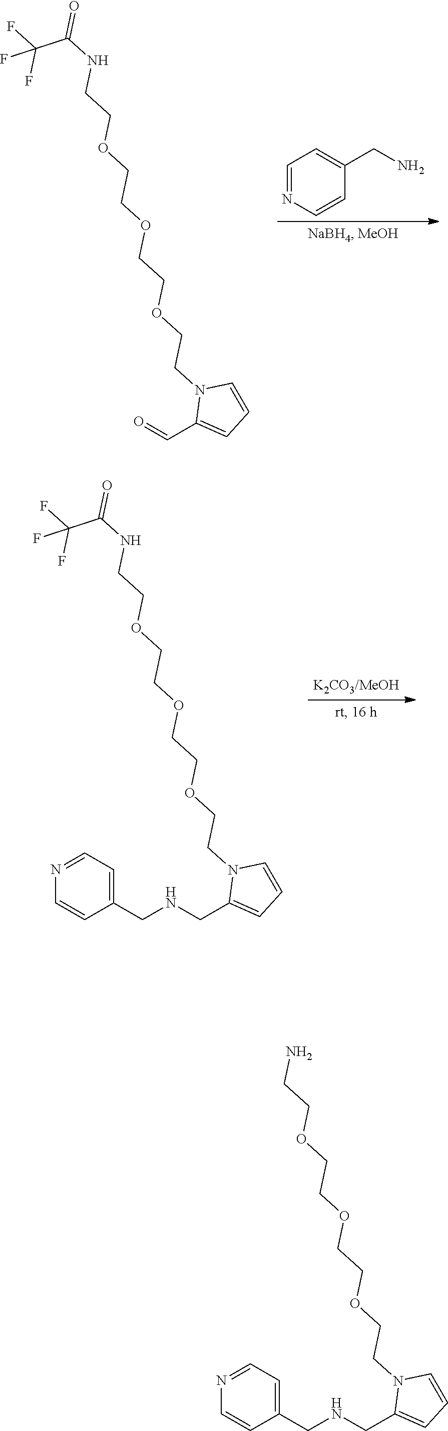 [SMILES: O=Cc1cccn1CCOCCOCCOCCNC(=O)C(F)(F)F ; NCc1ccncc1 ; O=C(NCCOCCOCCOCCn1cccc1CNCc1ccncc1)C(F)(F)F ; NCCOCCOCCOCCn1cccc1CNCc1ccncc1]

CPD-006 was synthesized following the standard procedure for preparing CPD-005 (16.6 mg, yield: 46%) as pale-yellow oil. MS (ESI) m/z=363.5 [M+H]⁺. ¹ H NMR (400 MHz, DMSO-d₆) δ 8.48 (d, J=5.6 Hz, 2H), 7.34 (d, J=5.2 Hz, 2H), 6.71 (s, 1H), 5.88-5.86 (m, 2H), 4.06 (t, J=5.6 Hz, 2H), 3.69 (s, 2H), 3.65-3.62 (m, 4H), 3.46-3.36 (m, 10H), 2.64 (t, J=5.2 Hz, 2H).

### Example 7: 3-Fluoro-N-((2-methyl-1,2,3,4-tetrahydroisoquinolin-3-yl)methyl)pyridin-2-amine (CPD-007)

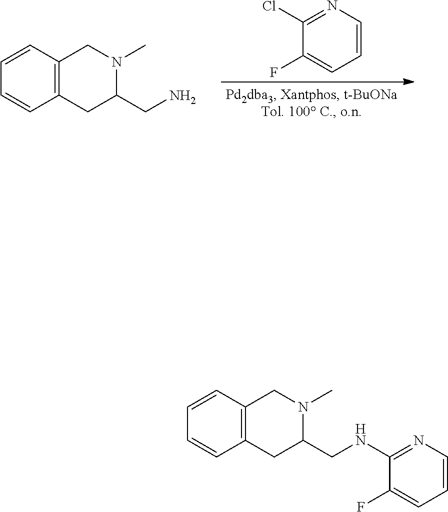 [SMILES: CN1Cc2ccccc2CC1CN ; Fc1cccnc1Cl ; CN1Cc2ccccc2CC1CNc1ncccc1F]

A solution of (2-methyl-1,2,3,4-tetrahydroisoquinolin-3-yl)methanamine (100 mg, 0.567 mmol), 2-chloro-3-fluoropyridine (75 mg, 0.567 mmol), Pd₂(dba)₃ (52 mg, 0.0567 mmol), Xantphos (33 mg, 0.0567 mmol) and t-BuONa (109 mg, 1.13 mmol) in toluene (10 mL) was stirred at 100° C. overnight under argon atmosphere. After being cooled to rt, the mixture was concentrated under reduced pressure. The residue was purified by prep-HPLC (0.1% FA) to give the title compound (21.0 mg, yield: 11.7%) as a colorless oil. ¹H NMR (400 MHz, DMSO-d₆) δ 8.16 (s, 1H), 7.81-7.80 (m, 1H), 7.35-7.30 (m, 1H), 7.13-7.04 (m, 4H), 6.52-6.48 (m, 1H), 6.46-6.44 (m, 1H), 3.83-3.79 (m, 1H), 3.65-3.56 (m, 2H), 3.35-3.29 (m, 1H), 3.06-3.00 (m, 1H), 2.85-2.79 (m, 1H), 2.73-2.67 (m, 1H), 2.41 (s, 3H). MS (ESI) m/z=272.2 [M+H]⁺.

### Example 8: 2-(2-(1H-1,2,3-Triazol-5-yl)ethyl)-8-chloro-2,3,4,5-tetrahydro-1H-pyrido[4,3-b]indole (CPD-008)

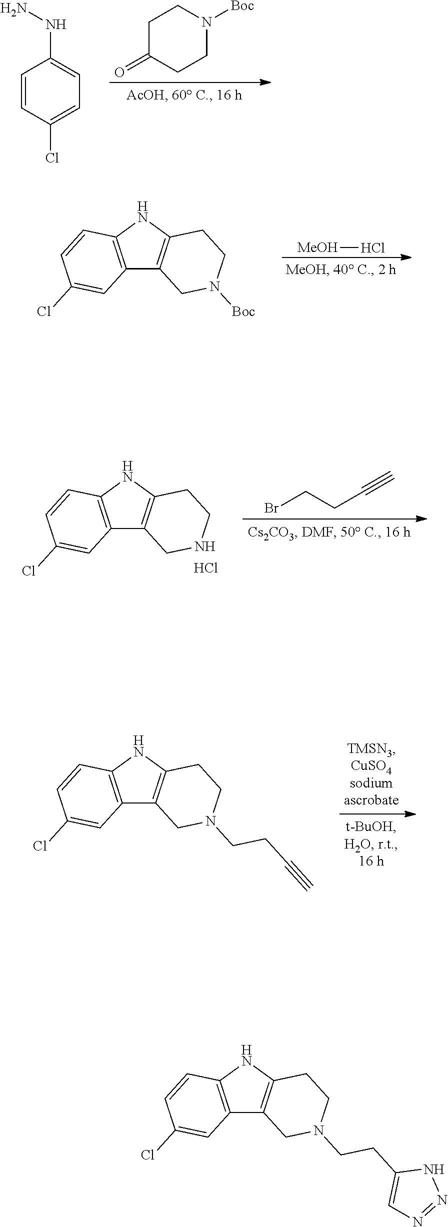

Step 1. Synthesis of tert-butyl 8-chloro-1,3,4,5-tetrahydro-2H-pyrido[4,3-b]indole-2-carboxylate

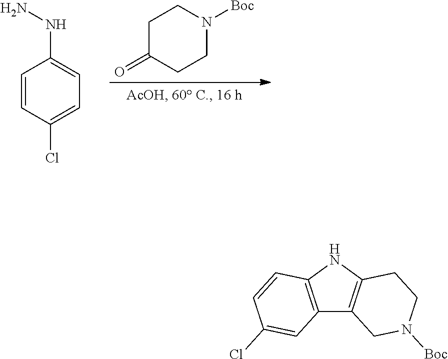

To a solution of (4-chlorophenyl)hydrazine hydrochloride (2.00 g, 11.2 mmol) in AcOH (20 mL) was added tert-butyl 4-oxopiperidine-1-carboxylate (2.67 g, 13.4 mmol) at rt under argon atmosphere. The mixture was heated to 60° C. for 16 h, before the mixture was diluted with H2O (30 mL) and extracted with EtOAc. The combined organic phase was washed with brine, dried over Na₂SO₄, filtered and concentrated under reduced pressure. The residue was purified by silica gel column chromatography (petroleum ether:EtOAc=3:1) to give the title compound (780 mg, 23.0% yield) as a yellow solid. MS (ESI) m/z=307.4 [M+H]⁺.

Step 2. Synthesis of 8-chloro-2,3,4,5-tetrahydro-1H-pyrido[4,3-b]indole

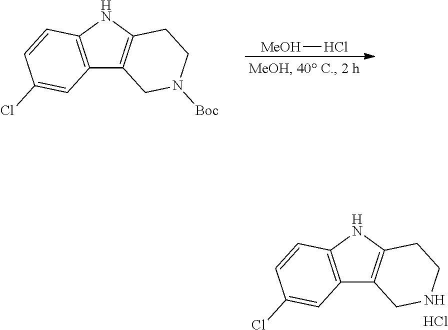

To a solution of tert-butyl 8-chloro-1,3,4,5-tetrahydro-2H-pyrido[4,3-b]indole-2-carboxylate (780 mg, 2.54 mmol) in MeOH (10 mL) was added HCl in MeOH (10 mL, 3 M) at rt. After the mixture was stirred at 40° C. for 2 h, the reaction mixture was concentrated to give the crude product (720 mg, 100% yield) as a yellow solid which was used directly in the next step. MS (ESI) m/z=207.4 [M+H]+.

Step 3. Synthesis of 2-(but-3-yn-1-yl)-8-chloro-2,3,4,5-tetrahydro-1H-pyrido [4,3-b]indole

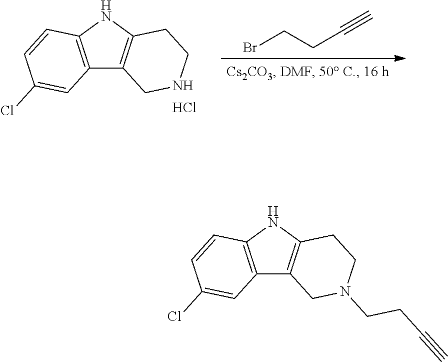

To a solution of 8-chloro-2,3,4,5-tetrahydro-1H-pyrido[4,3-b]indole hydrochloride (360 mg, 1.48 mmol) in DMF (10 mL) was added Cs₂CO₃ (296 mg, 2.23 mmol). After the reaction was heated at 50° C. for 16 h, the reaction mixture was cooled to rt, diluted with H₂O (20 mL) and extracted with EtOAc. The combined organic phase was washed with brine, dried over Na2SO4, filtered and concentrated under reduced pressure. The residue was purified by silica gel column chromatography (petroleum ether:EtOAc=3:1) to give the title compound (290 mg, 76.0% yield) as a yellow oil. MS (ESI) m/z=259.1 [M+H]⁺.

Step 4. Synthesis of 2-(2-(1H-1,2,3-triazol-5-yl)ethyl)-8-chloro-2,3,4,5-tetrahydro-1H-pyrido[4,3-b]indole

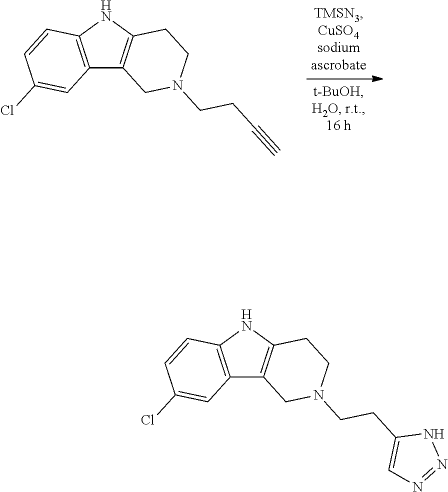 [SMILES: C#CCCN1CCc2[nH]c3ccc(Cl)cc3c2C1 ; Clc1ccc2[nH]c3c(c2c1)CN(CCc1cnn[nH]1)CC3]

To a solution of 2-(but-3-yn-1-yl)-8-chloro-2,3,4,5-tetrahydro-1H-pyrido[4,3-b]indole (290 mg, 1.12 mmol) in t-BuOH/water (10 mL/5 mL) was added CuSO₄ (178 mg, 1.12 mmol), TMSN₃ (129 mg, 1.12 mmol), sodium ascorbate (221 mg, 1.12 mmol) at rt. After the mixture was stirred at rt for 16 h, the reaction mixture was diluted with H₂O (20 mL) and filtered to give crude product which was further purified by prep-HPLC (0.1% TFA) to give the title compound (80 mg, 23.7% yield) as a yellow solid. MS (ESI) m/z=302.4 [M+H]⁺. ¹H NMR (400 MHz, DMSO-d₆) δ 11.46 (s, 1H), 10.11 (s, 1H), 7.80 (br s, 1H), 7.56 (d, J=1.6 Hz, 1H), 7.38 (d, J=8.4 Hz, 1H), 7.10-7.12 (m, 1H), 4.77-4.75 (m, 1H), 4.53 (br s, 1H), 3.85 (br s, 1H), 3.58-3.60 (m, 3H), 3.25-3.14 (m, 4H).

### Example 9: 2-(2-(1H-1,2,3-Triazol-5-yl)ethyl)-8-isopropyl-2,3,4,5-tetrahydro-1H-pyrido[4,3-b]indole (CPD-009)

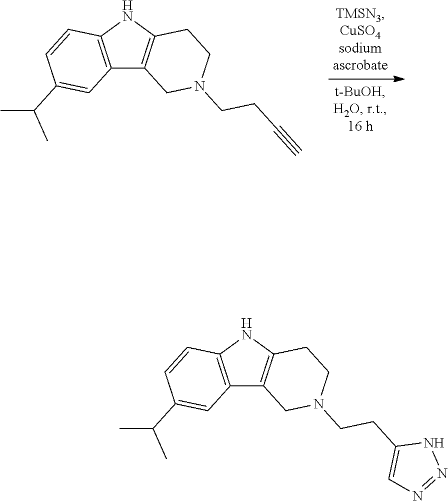

CPD-009 was synthesized following the standard procedure for preparing CPD-008 (20 mg, yield: 4.3%) as white solid. MS (ESI) m/z=310.2 [M+H]⁺. ¹H NMR (400 MHz, DMSO-d₆) δ 10.59 (s, 1H), 7.66 (s, 1H), 7.17-7.15 (m, 2H), 6.88 (dd, J=1.6 Hz, 8.4 Hz, 1H), 6.07 (br s, 1H), 3.63 (s, 2H), 2.96-2.75 (m, 9H), 1.22 (d, J=6.8 Hz, 6H).

### Example 10: 2-(2-Isopropyl-1,2,3,4-tetrahydro-5H-pyrido[4,3-b]indol-5-yl)acetic acid (CPD-010)

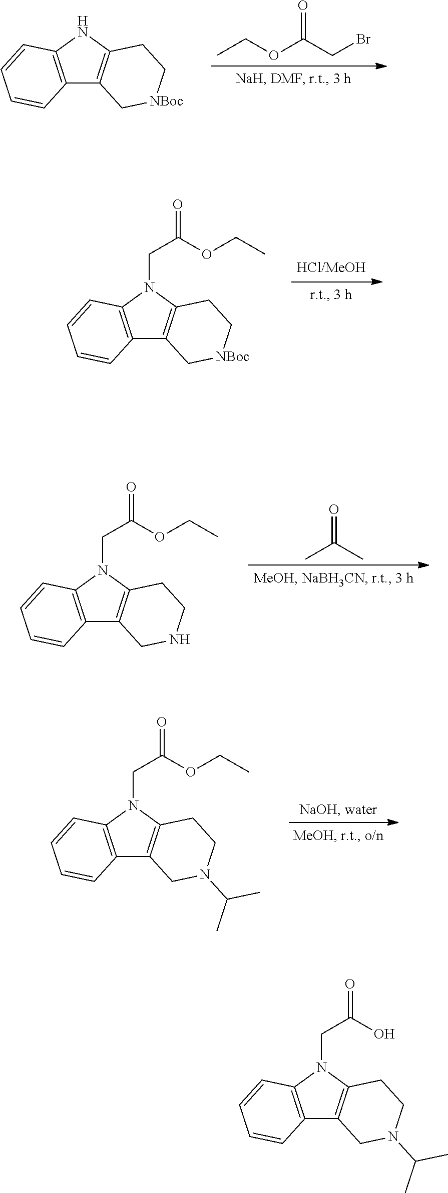

Step 1. Synthesis of tert-butyl 5-(2-ethoxy-2-oxoethyl)-1,3,4,5-tetrahydro-2H-pyrido[4,3-b]indole-2-carboxylate

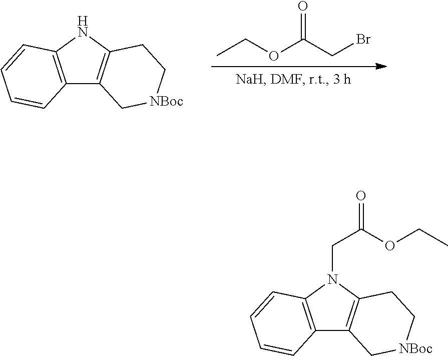

To a solution of tert-butyl 1,3,4,5-tetrahydro-2H-pyrido[4,3-b]indole-2-carboxylate (100 mg, 0.368 mmol) in DMF (2 mL) was added NaH (14.7 mg, 0.368 mmol) at rt under argon atmosphere. After the reaction was stirred for 0.5 hat rt, ethyl 2-bromoacetate (61.4 mg, 0.368 mmol) was added. The mixture was stirred at rt for 3 h, before the mixture was diluted with H₂O (20 mL) and extracted with EtOAc. The combined organic phase was washed with brine, dried over Na₂SO₄, filtered and concentrated under reduced pressure to give the title compound (110 mg, crude) which was used in the next step without further purification. MS (ESI) m/z=359.5 [M+H]⁺.

Step 2. Synthesis of ethyl 2-(1,2,3,4-tetrahydro-5H-pyrido[4,3-b]indol-5-yl)acetate

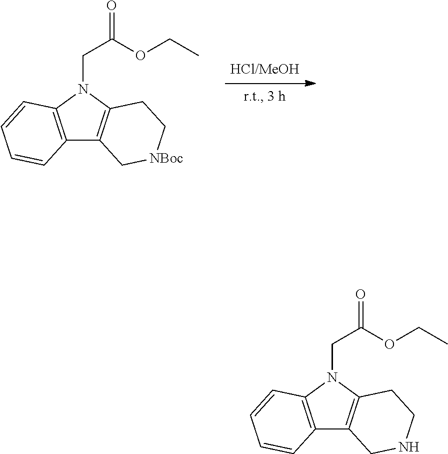 [SMILES: CCOC(=O)Cn1c2c(c3ccccc31)CN(C(=O)OC(C)(C)C)CC2 ; CCOC(=O)Cn1c2c(c3ccccc31)CNCC2]

To a solution of tert-butyl-5-(2-ethoxy-2-oxoethyl)-1,3,4,5-tetrahydro-2H-pyrido[4,3-b]indole-2-carboxylate (110 mg, crude) in MeOH (1 mL) was added methanol hydrochloride (3 mL, 2M) at rt. After the mixture was stirred at rt for 3 h, the mixture was concentrated under reduced pressure to give the title compound (98 mg, crude) as a yellow solid which was used in the next step without further purification. MS (ESI) m/z=259.2 [M+H]⁺.

Step 3. Synthesis of ethyl 2-(2-isopropyl-1,2,3,4-tetrahydro-5H-pyrido[4,3-b]indol-5-yl)acetate

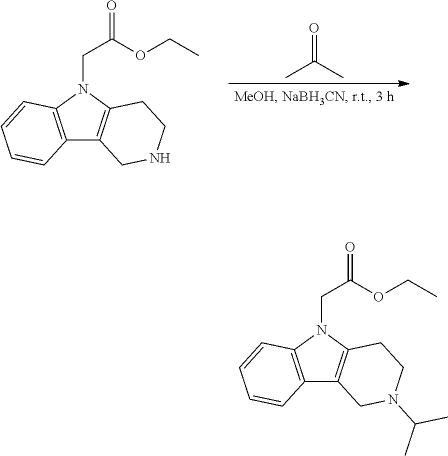

To a solution of ethyl 2-(1,2,3,4-tetrahydro-5H-pyrido[4,3-b]indol-5-yl)acetate (98 mg, crude) in MeOH (2 mL) was added acetone (2 mL) at rt under argon atmosphere. After the reaction was stirred at rt for 0.5 h, NaBH3CN (29 mg, 0.456 mmol) was added. The resulting mixture was stirred at rt for 3 h, before the mixture was diluted with H₂O (10 mL) and extracted with EtOAc. The combined organic phase was washed with brine, dried over Na₂SO₄, filtered and concentrated under reduced pressure to give the title compound (108 mg, crude) as a brown solid which was used in the next step without further purification. MS (ESI) m/z=301.6 [M+H]⁺.

Step 4. Synthesis of 2-(2-isopropyl-1,2,3,4-tetrahydro-5H-pyrido[4,3-b]indol-5-yl)acetic acid

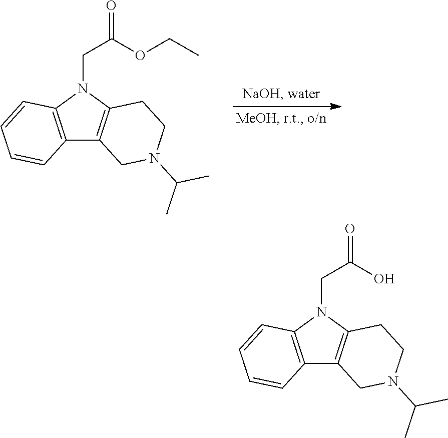 [SMILES: CCOC(=O)Cn1c2c(c3ccccc31)CN(C(C)C)CC2 ; CC(C)N1CCc2c(c3ccccc3n2CC(=O)O)C1]

To a solution of ethyl 2-(2-isopropyl-1,2,3,4-tetrahydro-5H-pyrido[4,3-b]indol-5-yl)acetate (108 mg, crude) in MeOH (10 mL) was added NaOH (10 N, 3 mL) at rt. After the mixture was stirred at rt overnight, the reaction mixture was purified by prep-HPLC (0.1% formic acid) to give the title compound (65.8 mg, 65.7% yield over three steps) as a white solid. MS (ESI) m/z=273.5 [M+H]⁺. ¹H NMR (400 MHz, DMSO-d6) δ 13.17 (br s, 1H), 9.78 (s, 1H), 7.54-7.44 (m, 2H), 7.18-7.06 (m, 2H), 5.07-4.95 (m, 2H), 4.56-4.52 (m, 1H), 4.38-4.36 (m, 1H), 3.84-3.69 (m, 2H), 3.44-3.39 (m, 1H), 3.10 (s, 2H), 1.40-1.37 (m, 6H).

### Example 11: 2-(2-(1H-1,2,3-Triazol-5-yl)ethyl)-8-(tert-butyl)-2,3,4,5-tetrahydro-1H-pyrido[4,3-b]indole (CPD-011)

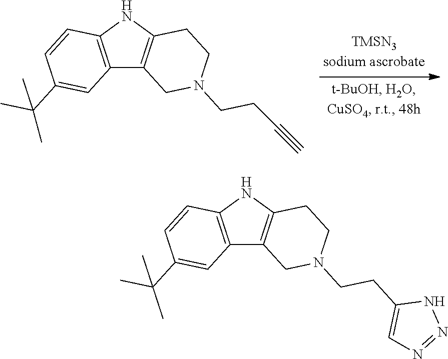

CPD-011 was synthesized following the standard procedure for preparing CPD-008 (19 mg, yield: 3.9%) as white solid. MS (ESI) m/z=324.5 [M+H]⁺. ¹H NMR (400 MHz, DMSO-d₆) δ 10.62 (s, 1H), 8.14 (s, 1H), 7.67 (s, 1H), 7.30 (d, J=1.2 Hz, 1H), 7.17 (d, J=8.4 Hz, 1H), 7.08 (dd, J=8.4 Hz, 2.0 Hz, 1H), 3.74 (s, 2H), 2.99-2.96 (m, 2H), 2.92-2.90 (m, 4H), 2.80-2.78 (m, 2H), 1.31 (s, 9H).

### Example 12: 2-(2-(1H-1,2,3-Triazol-5-yl)ethyl)-8-fluoro-2,3 ,4,5-tetrahydro-1H-pyrido[4,3-b]indole (CPD-012)

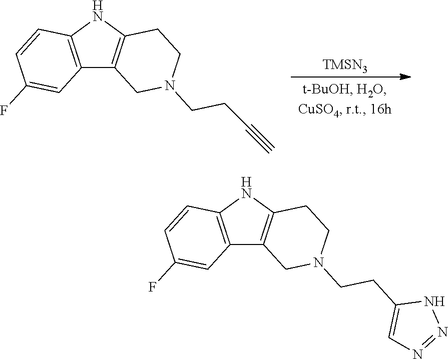 [SMILES: C#CCCN1CCc2[nH]c3ccc(F)cc3c2C1 ; "TMSN3" ; Fc1ccc2[nH]c3c(c2c1)CN(CCc1cnn[nH]1)CC3]

CPD-012 was synthesized following the standard procedure for preparing CPD-008 (27.6 mg, yield: 16.8%) as white solid. MS (ESI) m/z=286.4 [M+H]⁺. ¹H NMR (400 MHz, DMSO-d₆) δ 14.82 (br s, 1H), 11.34 (s, 1H), 10.02 (s, 1H), 8.02-7.74 (m, 1H), 7.37-7.34 (m, 1H), 7.28-7.25 (m, 1H), 6.97-6.92 (m, 1H), 4.75-4.70 (m, 1H), 4.36 (s, 1H), 3.91-3.85 (m, 1H), 3.60-3.32 (m, 3H), 3.15-3.10 (m, 2H), 3.08 (s, 2H).

### Example 13: 2-(2-(1H-1,2,3-Triazol-5-yl)ethyl)-8-methyl-2,3,4,5-tetrahydro-1H-pyrido[4,3-b]indole (CPD-013)

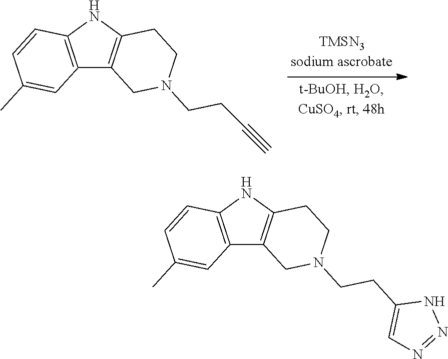

CPD-013 was synthesized following the standard procedure for preparing CPD-008 (6.7 mg, yield: 1.4%) as white solid. MS (ESI) m/z=282.1 [M+H]⁺. ¹H NMR (400 MHz, DMSO-d₆) δ 10.60 (s, 1H), 8.17 (s, 1H), 7.66 (s, 1H), 7.13 (d, J=8.0 Hz, 1H), 7.10 (s, 1H), 6.81 (d, J=8.0 Hz, 1H), 3.64 (s, 2H), 2.96-2.92 (m, 2H), 2.88-2.83 (m, 4H), 2.77-2.75 (m, 2H), 2.34 (s, 3H).

### Example 14: 6-(2-(2-(1H-1,2,3-Triazol-5-yl)ethyl)-8-fluoro-1,2,3,4-tetrahydro-5H-pyrido[4,3-b]indol-5-yl)hexan-1-amine (CPD-014)

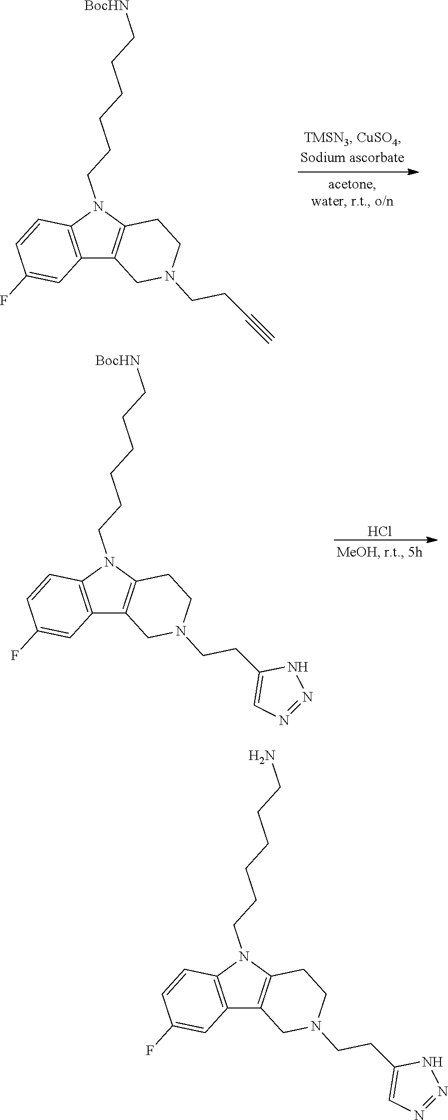

Step 1. Synthesis of tert-butyl (6-(2-(2-(1H-1,2,3-triazol-5-yl)ethyl)-8-fluoro-1,2,3,4-tetrahydro-5H-pyrido[4,3-b]indol-5-yl)hexylcarbamate

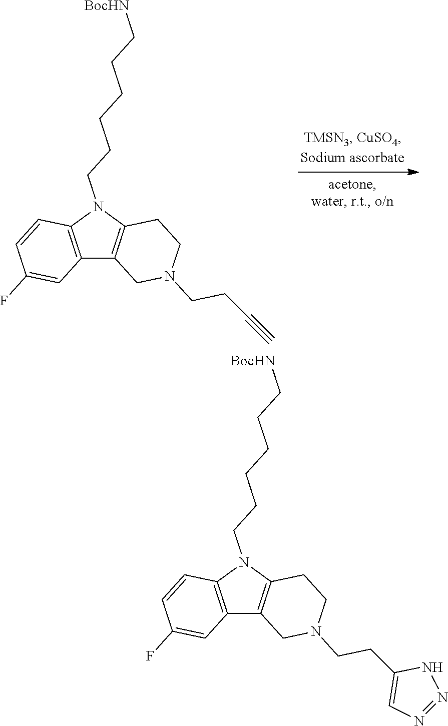

The title compound was synthesized following the standard procedure for preparing CPD-008 (176 mg, crude product) as brown oil which was used directly in the next step without further purification. MS (ESI) m/z=442.5 [M+H]⁺.

Step 2. Synthesis of 6-(2-(2-(1H-1,2,3-triazol-5-yl)ethyl)-8-fluoro-1,2,3,4-tetrahydro-5H-pyrido[4,3-b]indol-5-yl)hexan-1-amine

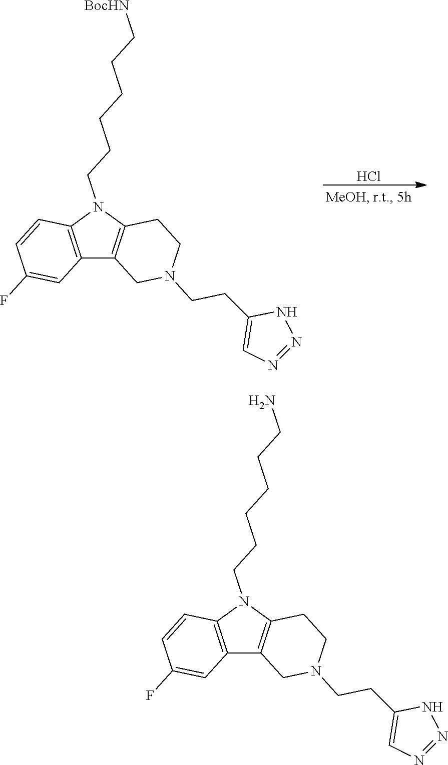

To a solution of tert-butyl (6-(2-(2-(1H-1,2,3-triazol-5-yl)ethyl)-8-fluoro-1,2,3,4-tetrahydro-5H-pyrido[4,3-b]indol-5-yl)hexylcarbamate (176 mg, crude) in MeOH (2 mL) was added HCl/MeOH (4 mL, 3 M) at rt. After the mixture was stirred at rt for 5 h, the reaction mixture was purified by prep-HPLC (0.1% FA) to give the title compound (9.5 mg, 5.5% yield) as a white solid. MS (ESI) m/z=385.5 [M+H]⁺. ¹H NMR (400 MHz, MeOD-d₄) δ 7.79 (s, 1H), 7.42-7.38 (m, 1H), 7.21-7.17 (m, 1H), 7.00-6.95 (m, 1H), 4.17-4.14 (m, 2H), 3.74-3.70 (m, 2H), 3.39-3.35 (m, 2H), 3.29-3.22 (m, 8H), 2.90-2.86 (m, 2H), 1.80-1.77 (m, 2H), 1.64-1.58 (m, 2H), 1.40-1.36 (m, 4H).

### Example 15: 6-Hydroxy-N-((2-methyl-1,2,3,4-tetrahydroisoquinolin-3-yl)methyl)pyrazine-2-carboxamide (CPD-015)

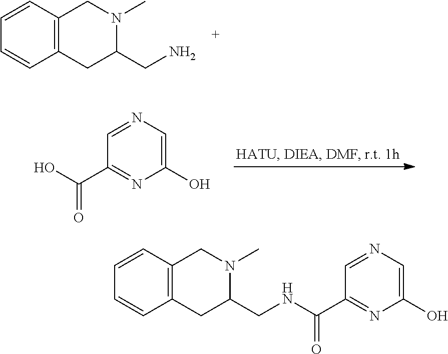 [SMILES: CN1Cc2ccccc2CC1CN ; O=C(O)c1cncc(O)n1 ; CN1Cc2ccccc2CC1CNC(=O)c1cncc(O)n1]

A solution of (2-methyl-1,2,3,4-tetrahydroisoquinolin-3-yl)methanamine (100 mg, crude), 6-hydroxypyrazine-2-carboxylic acid (80 mg, 0.568 mmol), HATU (432 mg, 1.14 mmol) and DIPEA (147 mg, 1.14 mmol) in DMF (10 mL) was stirred at rt for 1 h. After that, the mixture was purified by prep-HPLC (0.1% NH3·H2O) followed by prep-HPLC (0.1% FA) to give the title compound (3.91 mg, 2% yield) as a brown solid. MS (ESI) m/z=299.1 [M+H]⁺. ¹H NMR (400 MHz, DMSO-d₆) δ 8.46 (s, 1H), 8.35 (s, 1H), 8.22 (s, 1H), 7.13-7.15 (m, 4H), 3.83-3.79 (m, 3H), 2.91-2.69 (m, 4H), 2.37 (s, 3H).

### Example 16: 2-(2-(2-(2-(2-(2-(1H-1,2,3-Triazol-5-yl)ethyl)-8-fluoro-1,2,3,4-tetrahydro-5H-pyrido[4,3-b]indol-5yl)ethoxy)ethoxy)ethoxy)ethan-1-amine (CPD-016)

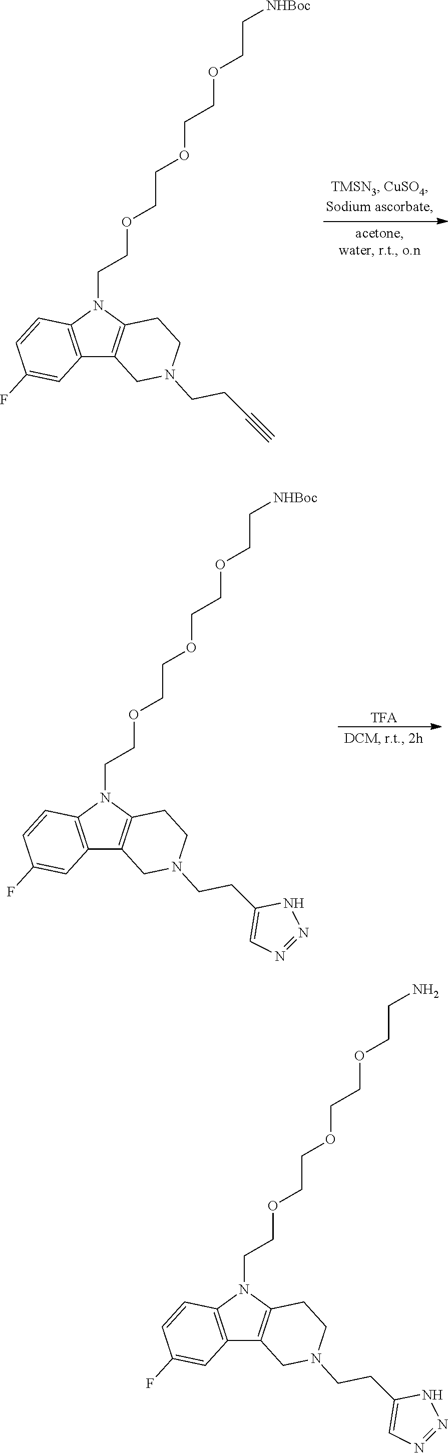 [SMILES: C#CCCN1CCc2c(c3cc(F)ccc3n2CCOCCOCCOCCNC(=O)OC(C)(C)C)C1 ; CC(C)(C)OC(=O)NCCOCCOCCOCCn1c2c(c3cc(F)ccc31)CN(CCc1cnn[nH]1)CC2 ; "TFA" ; NCCOCCOCCOCCn1c2c(c3cc(F)ccc31)CN(CCc1cnn[nH]1)CC2]

CPD-016 was synthesized following the standard procedure for preparing CPD-014 (105 mg, yield: 41.5%) as white solid. MS (ESI) m/z=461.6 [M+H]⁺. ¹H NMR (400 MHz, DMSO-d₆) δ 14.85 (s, 1H), 10.27 (s, 1H), 7.85-7.74 (m, 4H), 7.54-7.50 (m, 1H), 7.31-7.28 (m, 1H), 7.03-6.98 (m, 1H), 4.76-4.72 (m, 1H), 3.41-4.35 (m, 1H), 4.30-4.27 (m, 2H), 3.94-3.87 (m, 1H), 3.66-3.46 (m, 15H), 3.25-3.21 (m, 4H), 2.96-2.95 (m, 2H).

### Example 17: N-(2-(2-(2-(2-Aminoethoxy)ethoxy)ethoxy)ethyl)-3-(((3-fluoropyridin-2-yl)amino)methyl)-2-methyl-1,2,3,4-tetrahydroisoquinolin-7-amine (CPD-017)

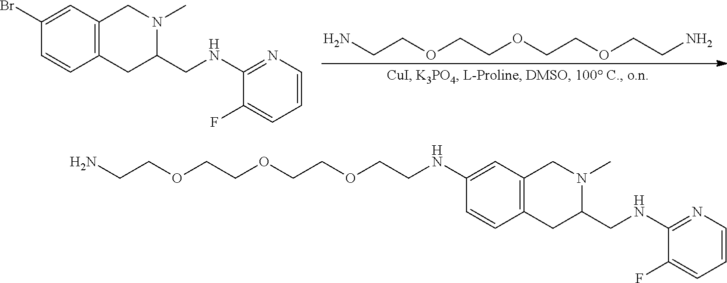

A solution of N-((7-bromo-2-methyl-1,2,3,4-tetrahydroisoquinolin-3-yl)methyl)-3-fluoropyridin-2-amine (50 mg, 0.143 mmol), 2,2′-((oxybis(ethane-2,1-diyl))bis(oxy))bis(ethan-1-amine) (137 mg, 0.715 mmol), L-proline (16 mg, 0.143 mmol), CuI (27 mg, 0.143 mmol) and K₃PO₄ (60 mg, 0.143 mmol) in DMSO (1 mL) was stirred at 100° C. overnight under argon atmosphere. After being cooled to rt, the mixture was purified by prep-HPLC (0.1% TFA) to give the tittle compound (8.60 mg, yield: 10.5%) as a white solid. MS (ESI) m/z=462.2 [M+H]⁺. ¹H NMR (400 MHz, DMSO-d₆) δ 10.18-10.10 (m, 1H), 7.86-7.85 (m, 4H), 7.44-7.39 (m, 1H), 6.97-6.95 (m, 2H), 6.65-6.59 (m, 2H), 6.38 (s, 1H), 4.60-4.44 (m, 1H), 4.26-4.16 (m, 1H), 3.76-3.48 (m, 15H), 3.18-3.15 (m, 2H), 2.99-2.82 (m, 7H).

### Example 18: N1-(3-(((3-Fluoropyridin-2-yl)amino)methyl)-2-methyl-1,2,3,4-tetrahydroisoquinolin-7-yl)hexane-1,6-diamine (CPD-018)

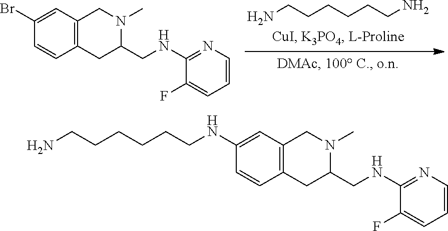

CPD-018 was synthesized following the standard procedure for preparing CPD-017 (8.2 mg, yield: 11.5%) as white solid. MS (ESI) m/z=386.1 [M+H]⁺. ¹H NMR (400 MHz, DMSO-d₆) δ 7.86-7.85 (m, 1H), 7.45-7.40 (m, 1H), 6.96 (d, J=8.4 Hz, 1H), 6.66-6.62 (m, 1H), 6.58-6.56 (m, 1H), 6.96 (d, J=1.2 Hz, 1H), 4.48-4.43 (m, 1H), 4.24-4.14 (m, 1H), 3.95-3.93 (m, 1H), 3.78-3.60 (m, 3H), 3.04-2.91 (m, 4H), 2.87-2.75 (m, 4H), 1.57-1.47 (m, 4H), 1.38-1.30 (m, 4H).

### Example 19: 2,8-Dimethyl-2,3,4,5-tetrahydro-1H-pyrido [4,3-b]indole (CPD-019)

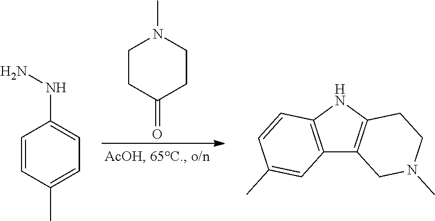

To a solution of p-tolylhydrazine (1.00 g, 6.30 mmol) in AcOH (10 mL) was added tert-butyl 1-methylpiperidin-4-one (0.85 g, 7.75 mmol) at rt under argon atmosphere. After the mixture was stirred at 65° C. overnight, the mixture was diluted with H₂O and extracted with EtOAc. The combined organic phase was washed with brine, dried over Na₂SO₄, filtered and concentrated under reduced pressure. The residue was purified by prep-HPLC (TFA) to give the title compound (534 mg, 42.4% yield) as a brown solid. MS (ESI) m/z=201.5 [M+H]⁺. ¹H NMR (400 MHz, DMSO-d₆) δ 11.08 (s, 1H), 10.09 (s, 1H), 7.24-7.22 (m, 1H), 7.18 (s, 1H), 6.94-6.91 (m, 1H), 4.59-4.56 (m, 1H), 4.26-4.23 (m, 1H), 3.73-3.70 (m, 2H), 3.11-3.06 (m, 2H), 2.99 (s, 3H), 2.36 (s, 3H).

### Example 20: 2,2,5,8-Tetramethyl-2,3,4,5-tetrahydro-1H-pyrido[4,3-b]indol-2-ium (CPD-020)

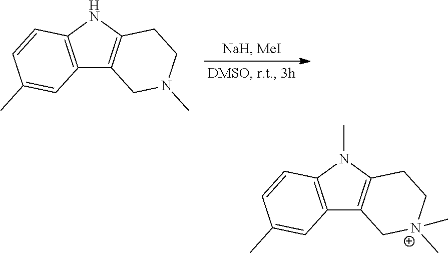

To a solution of 2,8-dimethyl-2,3,4,5-tetrahydro-1H-pyrido[4,3-b]indole (50 mg, 0.25 mmol) in DMSO (3 mL) was added NaH (90 mg, 0.375 mmol) and MeI (50 mg , 0.30 mmol) at rt under argon atmosphere. After the mixture was stirred for 3 h at rt, the mixture was purified by prep-HPLC (0.1% TFA) to give the title compound (7.6 mg, 13.3% yield) as a white solid. MS (ESI) m/z=229.5 [M+H]+. ¹H NMR (400 MHz, DMSO-d₆) δ 7.29 (d, J=8.4 Hz, 1H), 7.22 (s, 1H), 7.07-7.05 (m, 1H), 4.68 (s, 2H), 3.84-3.81 (m, 2H), 3.69 (s, 3H), 3.27-3.26 (m, 6H), 3.24 (s, 2H), 2.41 (s, 3H).

### Example 21: 2,2,8-Trimethyl-2,3,4,5-tetrahydro-1H-pyrido[4,3-b]indol-2-ium (CPD-021)

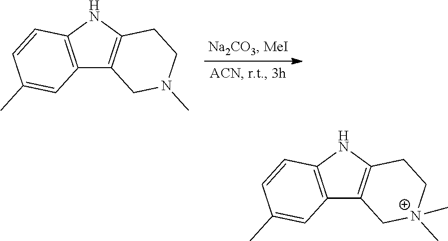

To a solution of 2,8-dimethyl-2,3,4,5-tetrahydro-1H-pyrido[4,3-b]indole (100 mg, 0.50 mmol) in acetonitrile (3 mL) was added Na₂CO₃ (130 mg, 0.75 mmol) and MeI (50 mg , 0.50 mmol) at rt under argon atmosphere. After the mixture was stirred for 3 h at rt, the mixture was purified by prep-HPLC (0.1% TFA) to give the title compound (78.6 mg, 73.1% yield) as a brown solid. MS (ESI) m/z=215.6 [M+H]+. 1H NMR (400 MHz, DMSO-d6) δ 11.20 (s, 1H), 7.26-7.24 (m, 1H), 7.16 (s, 1H), 6.95-6.93 (m, 1H), 4.63 (s, 2H), 3.75-3.72 (m, 2H), 3.19-3.16 (m, 6H), 3.15 (s, 2H), 2.36 (s, 3H).

### Example 22: 2,5,8-Trimethyl-2,3,4,5-tetrahydro-1H-pyrido [4,3-b]indole (CPD-022)

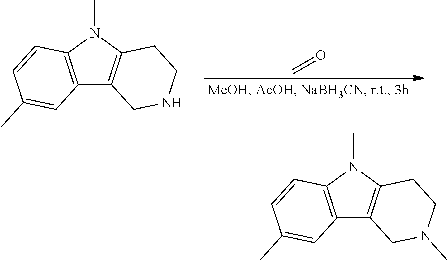

To a solution of tert-butyl 5,8-dimethyl-1,3,4,5-tetrahydro-2H-pyrido[4,3-b]indole-2-carboxylate (120 mg, crude) in MeOH (2 mL) was added formaldehyde/water (30%, 0.5 mL), AcOH (1 drop) at rt under argon atmosphere. After the reaction was stirred at rt for 30 min, NaBH₃CN (130 mg, 2.10 mmol) was added. After the mixture was stirred at rt for 3 h, the mixture was diluted with H₂O and extracted with EtOAc. The combined organic phase was washed with brine, dried over Na₂SO₄, filtered and concentrated under reduced pressure. The residue was purified by prep-HPLC (0.1% TFA) to give the title compound (29.8 mg, 41.8% yield) as brown solid. MS (ESI) m/z=215.6 [M+H]+. 1H NMR (400 MHz, DMSO-d₆) δ 9.98-9.96 (m, 1H), 7.36-7.34 (m, 1H), 7.21 (s, 1H), 7.01-7.99 (m, 1H), 4.62-4.57 (m, 1H), 4.27-4.22 (m, 1H), 3.81-3.80 (m, 1H), 3.72-3.67 (m, 1H), 3.64 (s, 3H), 3.16-3.09 (m, 2H), 2.99-2.98 (m, 2H), 2.37 (s, 3H).

### Example 23: Methyl (2-(3,4-dihydroisoquinolin-2(1H)-yl)ethyl)carbamate (CPD-023)

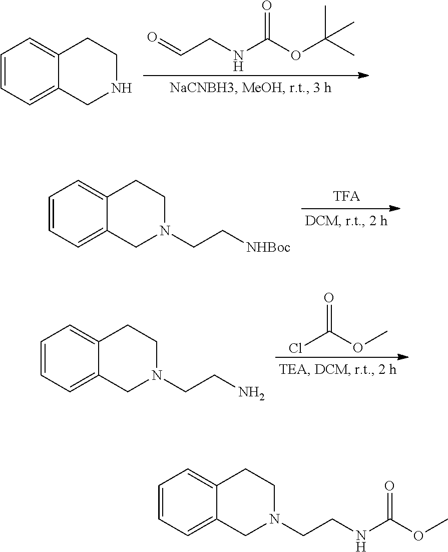

Step 1. Synthesis of tert-butyl (2-(3,4-dihydroisoquinolin-2(1H)-yl)ethyl)carbamate

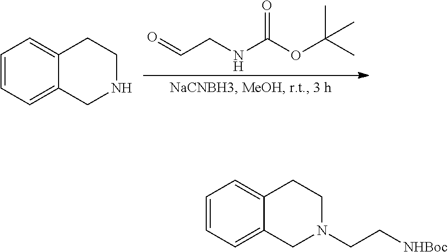

To a solution of 1,2,3,4-tetrahydroisoquinoline (200 mg, 1.50 mmol) in MeOH (10 mL) was added tert-butyl (2-oxoethyl)carbamate (239 mg, 1.50 mmol) and AcOH (1 drop) at rt under argon atmosphere. After the reaction was stirred at rt for 0.5 h, NaBH3CN (280 mg, 4.50 mmol) was added. After the mixture was stirred at rt for another 3 h, the mixture was diluted with H2O and extracted with EtOAc. The combined organic phase was washed with brine, dried over Na2SO4, filtered and concentrated under reduced pressure. The residue was purified by prep-HPLC (CH3CN:H20=40:1) to give the title compound (120 mg, 29.0% yield) as an oil. MS (ESI) m/z=277.1 [M+H]+.

Step 2. Synthesis of 2-(3,4-dihydroisoquinolin-2(1H)-yl)ethan-1-amine

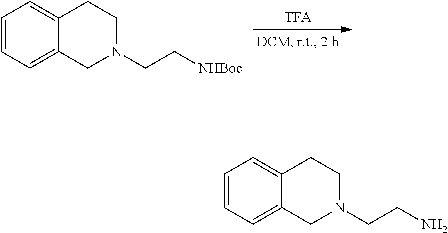 [SMILES: CC(C)(C)OC(=O)NCCN1CCc2ccccc2C1 ; NCCN1CCc2ccccc2C1]

To a solution of tert-butyl (2-(3,4-dihydroisoquinolin-2(1H)-yl)ethyl)carbamate (110 mg, 0.385 mmol) in DCM (4 mL) was added TFA (2 mL) at rt. After the mixture was stirred at rt for 2 h, the mixture was concentrated under reduced pressure to give the title compound (100 mg, crude) as brown solid which was used directly in the next step without further purification.

Step 3. Synthesis of methyl (2-(3,4-dihydroisoquinolin-2(1H)-yl)ethyl)carbamate

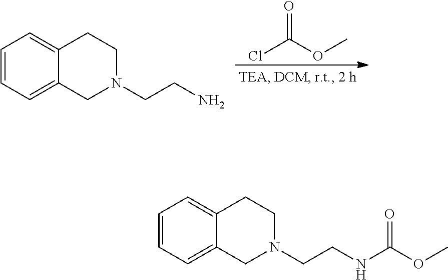

To a solution of 2-(3,4-dihydroisoquinolin-2(1H)-yl)ethan-1-amine (100 mg, crude) in DCM (2 mL) was added TEA (1 mL) and methyl carbonochloridate (61 mg, 0.645 mmol) at rt. After the mixture was stirred at rt for 2 h, the reaction mixture was purified by prep-HPLC (0.1% TFA) to give the title compound (32.6 mg, 9.3% three step yield) as a white solid. MS (ESI) m/z=235.5 [M+H]⁺. ¹H NMR (400 MHz, DMSO-d6) δ 7.32-7.25 (m, 3H), 7.20-7.19 (m, 1H), 4.61-4.43 (m, 2H), 3.86-3.77 (m, 7H), 3.65-3.58 (m, 2H), 3.20 (s, 2H).

### Example 24: N-((1H-Imidazol-5-yl)methyl)-1-(5-methylfuran-2-yl)methanamine, trifluoroacetic acid salt (CPD-024)

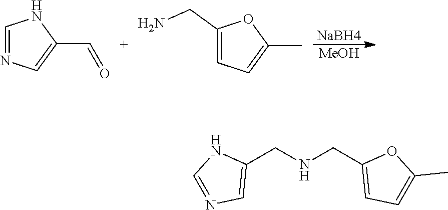

A mixture of 1H-imidazole-5-carbaldehyde (200 mg, 2.1 mmol) and (5-methylfuran-2-yl)methanamine (231 mg, 2.1 mmol) in MeOH (4 mL) was stirred at rt for 1 h, before NaBH₄ (95 mg, 2.5 mmol) was added. After the reaction was stirred at rt for 30 min, the mixture was concentrated and purified by prep-HPLC (0.1% TFA in H2O) to afford the title compound (328 mg, yield: 82%) as white solid. MS (ESI) m/z=192 [M+H]⁺. ¹H NMR (400 MHz, DMSO-d₆) δ 9.59 (br s, 1H), 8.71 (br s, 1H), 7.60 (s, 1H), 6.50 (d, J=2.8 Hz, 1H), 6.14 (t, J=2.0 Hz, 1H), 4.24 (s, 2H), 4.22 (s, 2H), 2.28 (s, 3H).

### Example 25: N-((1H-Imidazol-5-yl)methyl)-1-(2-methoxypyridin-4-yl)methanamine (CPD-025)

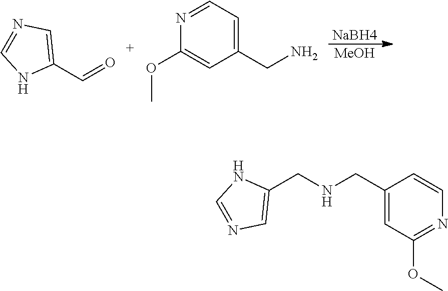

CPD-025 was synthesized following the standard procedure for preparing CPD-024 (243 mg, yield: 62%) as a colorless oil. MS (ESI) m/z=219.1 [M+H]⁺. ¹H NMR (400 MHz, DMSO-d₆) δ 9.81 (br s, 1H), 8.89 (s, 1H), 8.23 (d, J=5.2 Hz, 1H), 7.67 (s, 1H), 7.08 (dd, J=1.2, 5.2 Hz, 1H), 6.95 (s, 1H), 4.31 (s, 2H), 4.21 (s, 2H), 3.87 (s, 3H).

### Example 26: 4-((((1H-Imidazol-5-yl)methyl)amino)methyl)pyridin-2(1H)-one, hydrochloride acid salt (CPD-026)

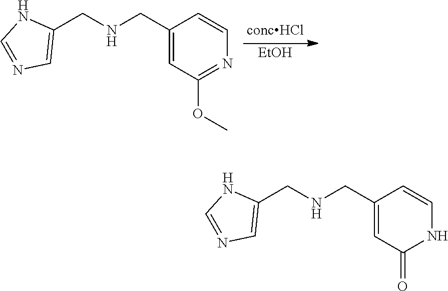 [SMILES: "EtOH" ; COc1cc(CNCc2cnc[nH]2)ccn1 ; O=c1cc(CNCc2cnc[nH]2)cc[nH]1]

To a solution of N-((1H-imidazol-5-yl)methyl)-1-(2-methoxypyridin-4-yl)methanamine (CPD-025, 50 mg, 0.229 mmol) in EtOH (5 mL) was added conc. HCl (12 M, 0.2 mL, 2.4 mmol). The mixture was stirred under refluxing for 18 h, before the mixture was concentrated and purified by prep-HPLC (0.1% HCl in H₂O) to afford the title compound (63 mg, yield: 99%) as a white solid. MS (ESI) m/z=205.1 [M+H]+. 1H NMR (400 MHz, DMSO-d6) δ 14.83 (br s, 1H), 10.51 (br s, 2H), 9.17 (d, J=0.8 Hz, 1H), 7.87 (s, 1H), 7.50 (d, J=6.8 Hz, 1H), 6.95 (s, 1H), 6.50 (d, J=6.8 Hz, 1H), 4.32 (s, 2H), 4.06 (s, 2H).

### Example 27: N-(1H-Imidazol-5-yl)methyl)-1-(5-methoxypyridin-3-yl)methanamine (CPD-027)

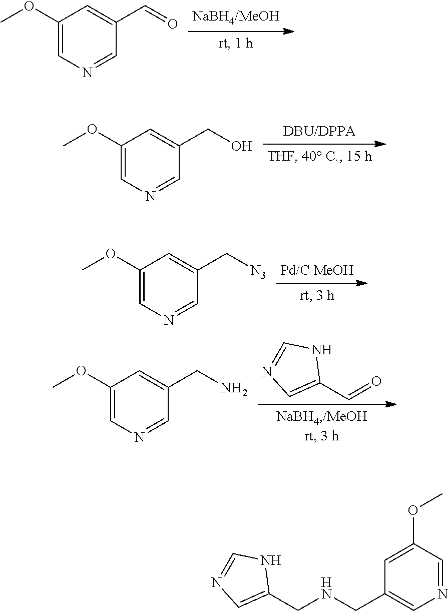

Step 1. Synthesis of (5-methoxvpyridin-3-yl)methanol

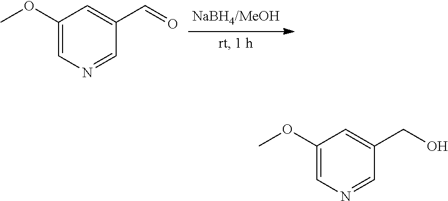

To a solution of 5-methoxynicotinaldehyde (1.0 g, 7.3 mmol) in THF (10 mL) was added NaBH₄ (552 mg, 15 mmol). After the reaction was stirred at rt for 1 h, the mixture was concentrated. The residue was redissolved in EtOAc. And the resulting solution was washed with H₂O and brine, dried over Na2SO4, and concentrated to afford the title compound (698 mg, yield: 70%) as white solid. 1H NMR (400 MHz, CDCl₃) δ 8.19 (s, 1H), 8.11 (s, 1H), 7.48 (s, 1H), 4.73 (s, 2H), 3.92 (s, 3H).

Step 2. Synthesis of 3-(azidomethyl)-5-methoxypyridine

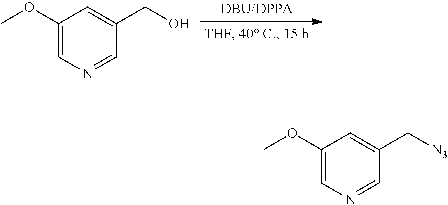

To a mixture of (5-methoxypyridin-3-yl)methanol (350 mg, 2.5 mmol) and DBU (570 mg, 4.5 mmol) in THF (7 mL) was added DPPA (825.6 mg, 3 mmol). After the mixture was stirred at 70° C. overnight, the mixture was diluted in EtOAc, washed with H₂O and brine, dried over Na2SO4, concentrated and purified by silica gel column (petroleum ether:EtOAc=20:1) to afford the title compound (150 mg, yield: 36%) as clear oil. ¹H NMR (400 MHz, CDCl₃) δ 8.19 (s, 1H), 8.16 (s, 1H), 7.41 (s, 1H), 4.49 (s, 2H), 3.94 (s, 3H).

Step 3. Synthesis of (5-methoxypyridin-3-yl)methanamine

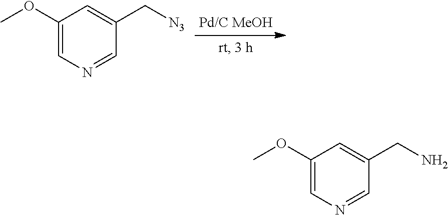

A mixture of 3-(azidomethyl)-5-methoxypyridine (140 mg, 0.85 mmol) and 5% Pd/C (wet, 16 mg) in MeOH (6 mL) was stirred at rt under H2 for 3 h. The mixture was filtered and concentrated to afford the title compound (104 mg, yield: 88%) as clear oil. MS (ESI) m/z=139.2 [M+H]⁺.

Step 4. Synthesis of N-((1H-imidazol-5-yl)methyl)-1-(5-methoxypyridin-3-yl)methanamine

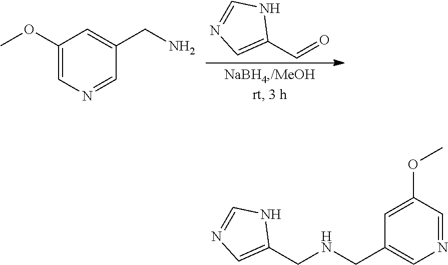

A mixture of 1H-imidazole-5-carbaldehyde (50 mg, 0.52 mmol) and (5-ethylpyridin-3-yl)methanamine (86.4 mg, 0.625 mmol) in MeOH (3 mL) was stirred at rt for 2 h, before NaBH₄ (29.5 mg, 0.78 mmol) was added. The resulting reaction mixture was stirred at rt for 1 h, before the mixture was concentrated and purified by prep-HPLC (0.1% NH₃·H₂O) to afford the title compound (18.4 mg, yield: 16%) as white solid. MS (ESI) m/z=219.4 [M+H]⁺. ¹H NMR (400 MHz, CDCl₃) δ8.22 (d, J=2.4 Hz, 1H), 8.12 (s, 1H), 7.57 (s, 1H), 7.23 (s, 1H), 6.89 (s, 1H), 3.83 (s, 3H), 3.79 (s, 4H).

### Example 28: N-((1H-Imidazol-5-yl)methyl)-1-(3-methoxy-5-methylphenyl)methanamine (CPD-028)

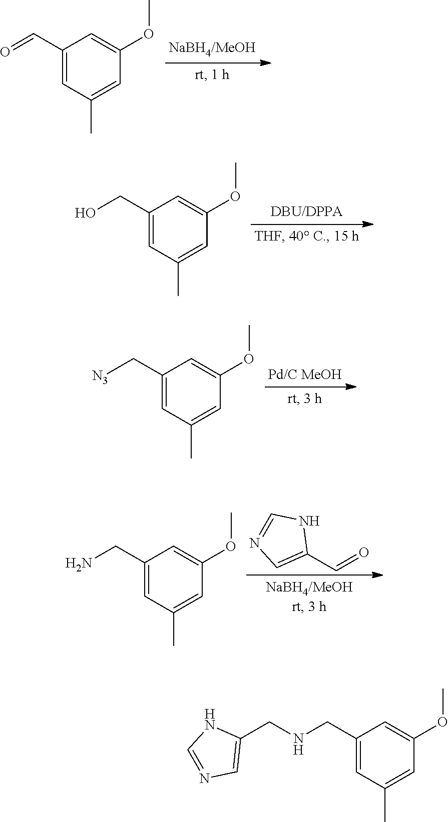

CPD-028 was synthesized following the standard procedure for preparing CPD-027 (20 mg, yield: 16%) as white solid. MS (ESI) m/z=232.4 [M+H]+. 1H NMR (400 MHz, CD3OD) δ 7.55 (s, 1H), 6.91 (s, 1H), 6.63 (s, 1H), 6.61 (s, 1H), 6.55 (s, 1H), 3.67 (s, 3H), 3.64 (s, 2H), 3.60 (s, 2H), 2.20 (s, 3H).

### Example 29: N-((1H-Imidazol-5-yl)methyl)-1-(1H-indol-6-yl)methanamine (CPD-029)

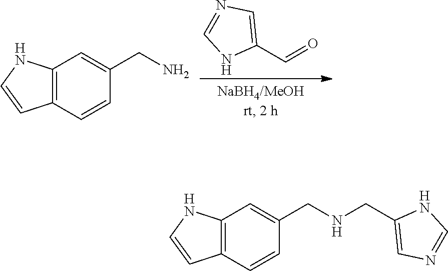 [SMILES: NCc1ccc2cc[nH]c2c1 ; O=Cc1cnc[nH]1 ; c1ncc(CNCc2ccc3cc[nH]c3c2)[nH]1]

CPD-029 was synthesized following the standard procedure for preparing CPD-001 (38 mg, yield: 48%) as white solid. MS (ESI) m/z=227.0 [M+H]⁺. ¹H NMR (400 MHz, DMSO-d₆) δ 10.99 (s, 1H), 7.54 (s, 1H), 7.45 (d, J=8.0 Hz, 1H), 7.34 (s, 1H), 7.28 (t, J=2.8 Hz, 1H), 6.96 (d, J=8.0 Hz, 1H), 6.85 (s, 1H), 6.37 (s, 1H), 3.77 (s, 2H), 3.61 (s, 2H).

### Example 30: N-((1H-Imidazol-5-yl)methyl)-1-(7-chlorobenzo [d][1,3]dioxol-5-yl)methanamine (CPD-030)

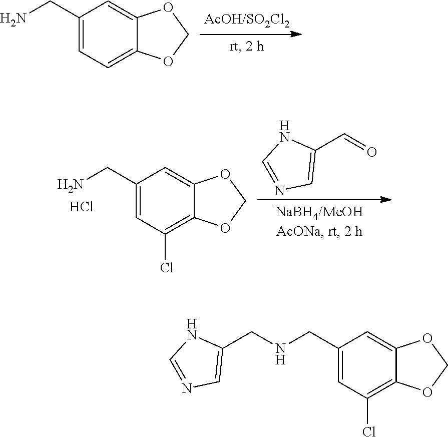

Step 1. Synthesis of (7-chlorobenzo[d][1,3]dioxol-5-yl)methanamine, hydrochloride

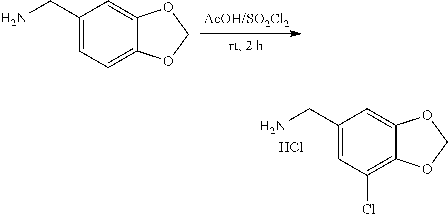 [SMILES: NCc1ccc2c(c1)OCO2 ; NCc1cc(Cl)c2c(c1)OCO2 ; Cl]

To a solution of benzo[d][1,3]dioxo1-5-ylmethanamine (2.0 g, 13.2 mmol) in AcOH (20 mL) was added SO₂Cl₂ (2.68 g, 19.8 mmol) dropwise in an ice bath. The mixture was stirred at rt for 2 h. The mixture was filtered, washed with ether to afford the title compound (1.8 g, yield: 61%) as white solid. 1H NMR (400 MHz, DMSO-d₆) δ 8.58 (br s, 3H), 7.27 (s, 1H), 7.17 (s, 1H), 6.11 (s, 2H), 4.01 (d, J=4.4 Hz, 2H).

Step 2. Synthesis of N-((1H-imidazol-5-yl)methyl)-1-(7-chlorobenzo[d][1,3]dioxol-5-yl)methanamine

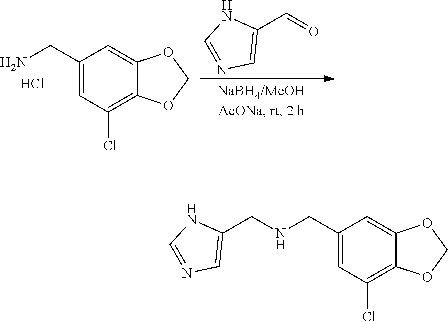

A mixture of (7-chlorobenzo [d][1,3]dioxo1-5-yl)methanamine hydrochloride (222 mg, 1 mmol), 1H-imidazole-5-carbaldehyde (96 mg, 1 mmol) and NaOAc (82 mg, 1 mmol) in MeOH (4 mL) was stirred at rt for 2 h, before NaBH₄ (38 mg, 1 mmol) was added. After the mixture was stirred at rt for 1 h, the mixture was concentrated and purified by prep-TLC (DCM:MeOH:NH3·H2O=10:1:0.1) to afford the title compound (29.7 mg, yield: 11%) as white solid. MS (ESI) m/z=266.0 [M+H]⁺. ¹H NMR (400 MHz, DMSO-d₆) δ 7.56 (s, 1H), 7.12 (s, 1H), 7.04 (s, 1H), 6.90 (s, 1H), 6.05 (s, 2H), 3.71 (s, 2H), 3.63 (s, 2H).

### Example 31: 2-(4-((((1H-Imidazol-4-yl)methyl)amino)methyl)phenoxy)ethan-1-ol (CPD-031)

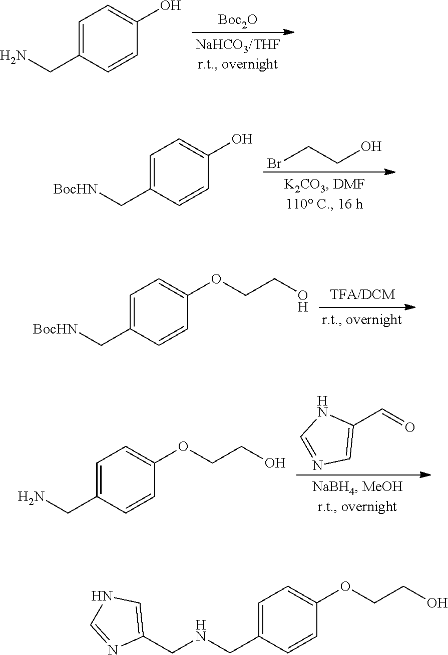

Step 1. Synthesis of tert-butyl (4-hydroxybenzyl)carbamate

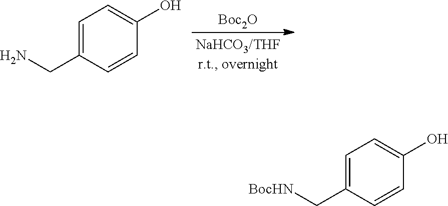 [SMILES: "Boc2O" ; NCc1ccc(O)cc1 ; CC(C)(C)OC(=O)NCc1ccc(O)cc1]

To a solution of 4-(aminomethyl)phenol (0.5 g, 4.06 mmol) in THF (10 mL) was added a solution of NaHCO₃ (1.02 g, 12.18 mmol) in H₂O (10 mL), followed by Boc₂O (975 mg, 4.47 mmol). After the resulting mixture was stirred at rt for 1 h, the solution was diluted with water (20 mL), and extracted with EtOAc (3×20 mL). The combined organic layers were washed with brine (50 mL), dried over sodium sulfate, filtered, concentrated and purified by silica gel chromatography (petroleum ether:EtOAc=2:1) to afford the title compound (820 mg, yield: 91%) as yellow oil. ¹H NMR (400 MHz, DMSO-d₆) δ 9.25 (s, 1H), 7.25 (t, J=6.0 Hz, 1H), 7.02 (d, J=8.4 Hz, 2H), 6.68 (d, J=8.4 Hz, 2H), 3.99 (d, J=6.0 Hz, 2H), 1.38 (s, 9H).

Step 2. Synthesis of tert-butyl (4-(2-hydroxyethoxy)benzyl)carbamate

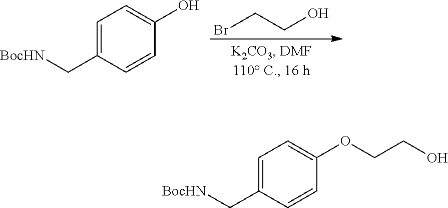

To a solution of tert-butyl 4-hydroxybenzylcarbamate (300 mg, 1.34 mmol) and K₂CO₃ (371 mg, 2.68 mmol) in DMF (5 mL) was added 2-bromoethanol (202 mg, 1.61 mmol). After the mixture was stirred at 110° C. for 16 h, the yellow mixture was diluted with water (20 mL) and extracted with EtOAc (3×20 mL). The combined organic layers were washed with brine (50 mL), dried over sodium sulfate, filtered, concentrated and purified by silica gel chromatography (petroleum ether:EtOAc=2:1) to afford the title compound (230 mg, yield: 64%) as yellow oil. ¹H NMR (400 MHz, DMSO-d₆) δ 7.31 (t, J=6.0 Hz, 1H), 7.13 (d, J=8.0 Hz, 2H), 6.89 (d, J=8.0 Hz, 2H), 4.85 (t, J=6.0 Hz, 1H), 4.04 (d, J=6.4 Hz, 2H), 3.94 (t, J=4.8 Hz, 2H), 3.71-3.67 (m, 2H), 1.38 (s, 9H).

Step 3. Synthesis of 2-(4-(aminomethyl)phenoxy)ethan-1-ol

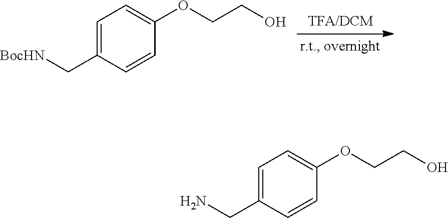 [SMILES: CC(C)(C)OC(=O)NCc1ccc(OCCO)cc1 ; NCc1ccc(OCCO)cc1]

To a solution of tert-butyl (4-(2-hydroxyethoxy)benzyl)carbamate (230 mg, 0.86 mmol) in DCM (2 mL) was added TFA (1 mL). The resulting mixture was stirred at 20° C. for 2 h, before the yellow solution was diluted with water (2 mL), basified to pH=7˜8 with sat. NaHCO₃ and concentrated. The residue was triturated with DCM:MeOH (10:1, 20 mL) and filtered. The filtrate was concentrated to afford the title compound (270 mg) as yellow oil, which was used directly in the next step. ¹H NMR (400 MHz, DMSO-d₆) δ 7.25 (d, J=8.8 Hz, 2H), 6.88 (d, J=8.8 Hz, 2H), 4.85 (br s, 1H), 4.15 (br s, 2H), 3.97-3.94 (m, 2H), 3.71 (s, 4H).

### Step 4. Synthesis of 2-(4-((((1H-imidazol-4-yl)methyl)amino)methyl)phenoxy)ethan-1-ol

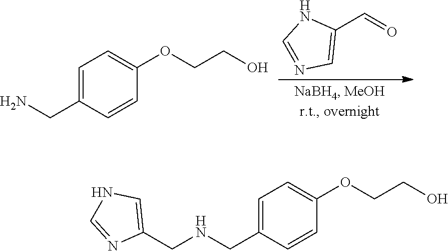

A mixture of 1H-imidazole-5-carbaldehyde (150 mg, 1.56 mmol) and 2-(4-(aminomethyl)phenoxy)ethan-1-ol (260 mg, 2.1 mmol) in MeOH (2 mL) was stirred at rt for 1 h, before NaBH₄ (60 mg, 1.56 mmol) was added. After the mixture was stirred at rt for 30 min, the mixture was concentrated and purified by prep-HPLC (0.1% NH₄HCO₃ in H₂O) to afford the title compound (12.6 mg, yield: 5%) as white solid. MS (ESI) m/z=248.4 [M+H]+. 1H NMR (400 MHz, CD3OD) δ 7.54 (s, 1H), 7.15 (d, J=8.4 Hz, 2H), 6.91 (s, 1H), 6.83 (d, J=8.4 Hz, 2H), 3.93 (t, J=7.6 Hz, 2H), 3.75 (t, J=7.6 Hz, 2H), 3.64 (s, 2H), 3.61 (s, 2H).

### Example 32 & 33: trans-2,8-Dimethyl-2,3,4,4a,5,9b-hexahydro-1H-pyrido[4,3-b]indole (CPD-032) and cis-2,8-dimethyl-2,3,4,4a,5,9b-hexahydro-1H-pyrido[4,3-b]indole (CPD-033)

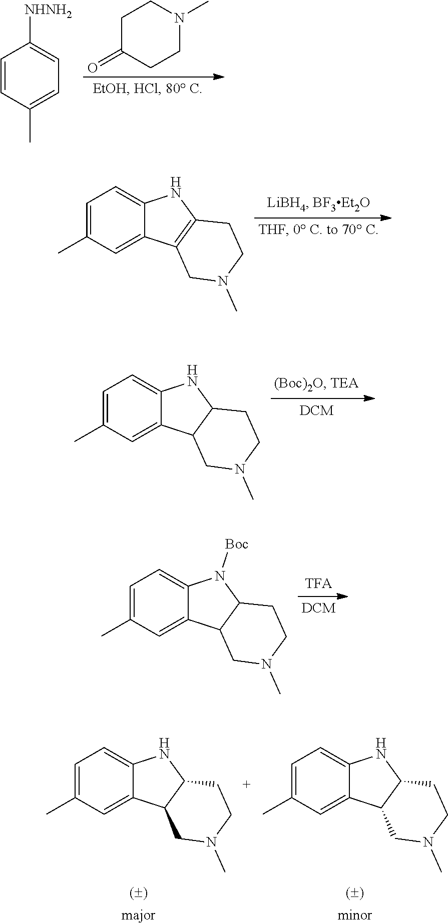

Step 1. Synthesis of 2,8-dimethyl-2,3,4,5-tetrahydro-1H-pyrido[4,3-b]indole

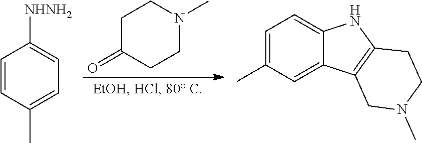

To a solution of p-tolylhydrazine hydrochloride (10 g, 63 mmol) and 1-methylpiperidin-4-one (7.1 g, 63 mmol) in EtOH (200 mL) was added 12 N HCl (26 mL, 315 mmol). The reaction mixture was stirred at 80° C. overnight. After removal of EtOH, the pH value was adjusted to pH=12 with NaOH (12 N). And the resulting mixture was extracted with DCM (3×100 mL). The combined organic layers were dried over Na2SO4, filtered, concentrated and purified by silica-gel chromatography (DCM/MeOH=20:1 to 10:1) to give the title compound (10.1 g, 80% yield) as white solid. MS (ESI) m/z=201.1 [M+H]⁺.

Step 2. Synthesis of 2,8-dimethyl-2,3,4,4a,5,9b-hexahydro-1H-pyrido[4,3-b]indole

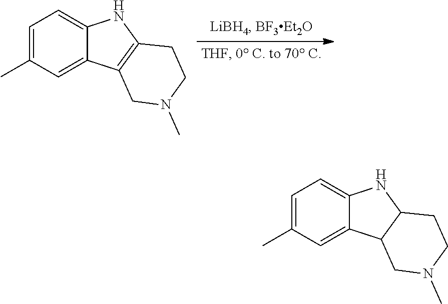

To a solution of 2,8-dimethyl-2,3,4,5-tetrahydro-1H-pyrido[4,3-b]indole (3 g, 15 mmol) in THF (100 mL) was added LiBH 4 in THF (2 M, 22.5 mL, 45 mmol) at 0° C., followed by BF₃·Et₂O (15.7 mL, 60 mmol). The reaction mixture was stirred at rt for 30 min and then at 70° C. for 6 h. HCl (6N, 25 mL) was added. The reaction mixture was stirred at 100° C. for 1 h. The pH value was adjusted to pH=10 with NaOH (6 N). After removal of the organic solvent, the residue was extracted with DCM (3×100 mL). The combined organic layers were washed with brine and dried over Na₂SO₄, filtered and concentrated under vacuum to give the title compound (3.03 g, crude) as yellow oil. MS (ESI) m/z=203.2 [M+H]⁺.

Step 3. Synthesis of tert-butyl 2,8-dimethyl-1,2,3,4,4a,9b-hexahydro-5H-pyrido[4,3-b]indole-5-carboxylate

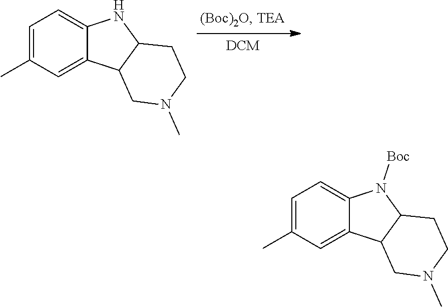 [SMILES: Cc1ccc2c(c1)C1CN(C)CCC1N2 ; Cc1ccc2c(c1)C1CN(C)CCC1N2C(=O)OC(C)(C)C]

To a solution of 2,8-dimethyl-2,3,4,4a,5,9b-hexahydro-1H-pyrido[4,3-b]indole (3.03 g, 15 mmol) in DCM (100 mL) was added TEA (3.78 g, 37.5 mmol) and (Boc)2O (4.9 g, 22.5 mmol). After the resulting mixture was stirred at rt overnight, the reaction was quenched with H₂O and extracted with DCM (3×50 mL). The combined organic layers were dried over N₂SO₄, filtered, concentrated and purified by silica-gel chromatography (DCM/MeOH=20:1) to give the title compound (2.04 g, 44% yield) as yellow oil. MS (ESI) m/z=303.2 [M+H]⁺.

Step 4. Synthesis of trans-2,8-dimethyl-2,3,4,4a,5,9b-hexahydro-1H-pyrido[4,3-b]indole and cis-2,8-dimethyl-2,3,4,4a,5,9b-hexahydro-1H-pyrido[4,3-b]indole

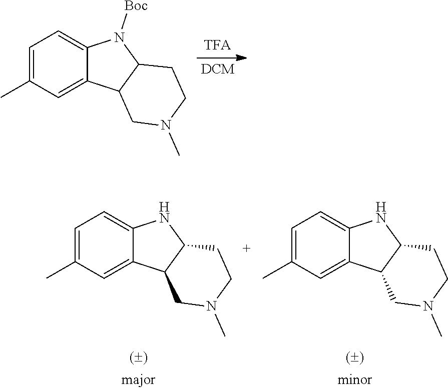

To a solution of tert-butyl 2,8-dimethyl-1,2,3,4,4a,9b-hexahydro-5H-pyrido[4,3-b]indole-5-carboxylate (1.8 g, 5.96 mmol) in DCM (50 mL) was added TFA (5 mL). The reaction mixture was stirred at rt for 1 h. After removal of the solvent, the residue was diluted with H₂O (5 mL), and the pH value was adjusted to pH=8 with aq. NaHCO₃. The resulting mixture was extracted with DCM (3×50 mL). The combined organic layers were dried over Na₂SO₄, filtered, concentrated and purified by silica-gel chromatography to give the major product trans-2,8-dimethyl-2,3,4,4a,5,9b-hexahydro-1H-pyrido[4,3-b]indole (1 g, 83% yield) as white solid and minor product cis-2,8-dimethyl-2,3,4,4a,5,9b-hexahydro-1H-pyrido[4,3-b]indole (60 mg, 5% yield) as yellow oil. Major: ¹H NMR (400 MHz, DMSO-d6) δ 6.80 (s, 1H), 6.76-6.73 (m, 1H), 6.50 (d, J=7.6 Hz, 1H), 5.52 (d, J=3.2 Hz, 1H), 3.35-3.32 (m, 1H), 2.87-2.78 (m, 2H), 2.61-2.54 (m, 1H), 2.28 (s, 3H), 2.17 (s, 3H), 2.05-1.99 (m, 2H), 1.92-1.88 (m, 1H), 1.76-1.69 (m, 1H), 2.16 (s, 3H), MS (ESI) m/z=203.1 [M+H]⁺. Minor: ¹H NMR (400 MHz, DMSO-d6) δ 6.83 (s, 1H), 6.72 (d, J=8.0 Hz, 1H), 6.43 (d, J=7.6 Hz, 1H), 5.22 (s, 1H), 3.61-3.57 (m, 1H), 2.99-2.94 (m, 1H), 2.49-2.47 (m, 1H), 2.30-2.25 (m, 1H), 2.22-2.19 (m, 1H), 2.16 (s, 3H), 2.10 (s, 3H), 2.00-1.97 (m, 1H), 1.80-1.73(m, 1H), 1.67-1.62(m, 1H).

MS (ESI) m/z=203.2 [M+H]⁺.

### Example 34: trans-2,5,8-Trimethyl-2,3,4,4a,5,9b-hexahydro-1H-pyrido[4,3-b]indole (CPD-034)

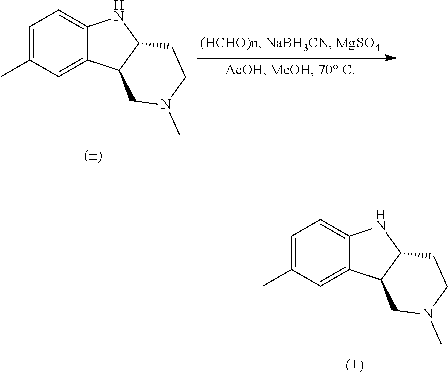

To a solution of trans-2,8-dimethyl-2,3,4,4a,5,9b-hexahydro-1H-pyrido[4,3-b]indole (250 mg, 1.24 mmol) and (HCHO)n (55 mg, 1.85 mmol) in MeOH (10 mL) was added MgSO₄ (100 mg, 0.83 mmol) and AcOH (2 drops). The reaction mixture was stirred at 70° C. overnight, before NaBH3CN (155.5 mg, 2.47 mmol) was added. The resulting mixture was stirred at rt. for another 1 h. The reaction was filtered, concentrated and purified by prep-HPLC to give the tile compound (29 mg, 11% yield) as white solid. ¹H NMR (400 MHz, DMSO-d6) δ 6.86 (d, J=7.6 Hz, 1H), 6.83 (s, 1H), 6.52 (d, J=7.6 Hz, 1H), 3.37-3.34 (m, 1H), 2.95-2.92 (m, 1H), 2.67-2.60 (m, 1H), 2.58 (s, 3H), 2.29 (s, 3H), 2.24-2.20 (m, 1H), 2.19 (s, 3H), 2.05-1.98 (m, 3H), 1.69-1.62 (m, 1H). MS (ESI) m/z=217.2 [M+H]⁺.

### Example 35: (E)-1-(trans-2,8-Dimethyl-1,2,3,4,4a,9b-hexahydro-5H-pyrido[4,3-b]indol-5-yl)-3-(thiazol-2-yl)prop-2-en-1-one (CPD-035)

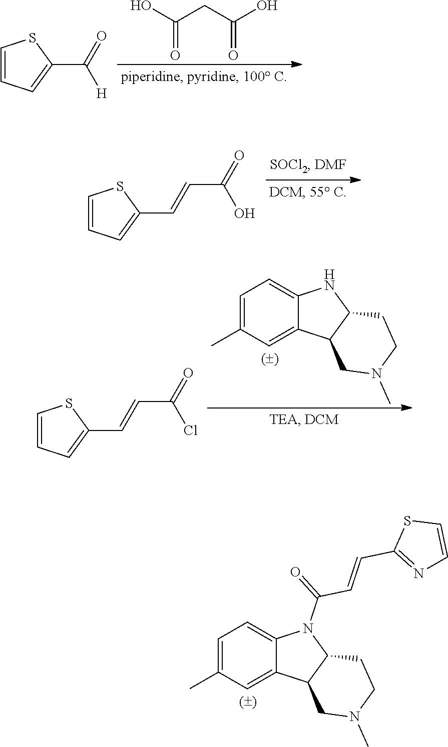

Step 1. Synthesis of (E)-3-(thiazol-2-yl)acrylic acid

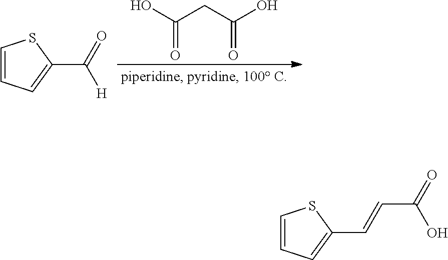 [SMILES: O=Cc1cccs1 ; O=C(O)CC(=O)O ; O=C(O)/C=C/c1cccs1]

To a solution of thiazole-2-carbaldehyde (1 g, 8.8 mmol) and malonic acid (0.92 g, 8.8 mmol) in pyridine (10 mL) was added piperidine (1 drop). The reaction mixture was stirred at 100° C. under N₂ atmosphere overnight. After removal of the solvent, the residue was diluted with water. The pH value was adjusted to pH=5 with HCl (2 N). The resulting mixture was filtered and the filter cake was dried to give the tile compound (1.37 g, 67% yield) as white solid. ¹H NMR (400 MHz, DMSO-d6) δ 12.79 (s, 1H), 8.00 (d, J=3.2 Hz, 1H), 7.93 (d, J=3.2 Hz, 1H), 7.69 (d, J=15.6 Hz, 1H), 6.67 (d, J=15.6 Hz, 1H).

Step 2. Synthesis of (E)-3-(thiazol-2-yl)acryloyl chloride

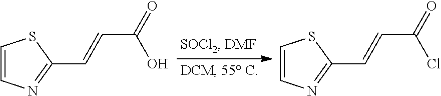 [SMILES: O=C(O)/C=C/c1nccs1 ; O=C(Cl)/C=C/c1nccs1]

To a solution of (E)-3-(thiazol-2-yl)acrylic acid (200 mg, 1.3 mmol) in DCM (10 mL) was added SOCl2 (0.27 mL, 3.9 mmol) and DMF (2 drops) at rt. The reaction mixture was stirred at 55° C. for 1 h, before being concentrated under vacuum to give the tile compound (223 mg, crude) as yellow oil.

Step 3. Synthesis of (E)-1-(trans-2,8-dimethyl-1,2,3,4,4a,9b-hexahydro-5H-pyrido[4,3-b]indol-5-yl)-3-(thiazol-2-yl)prop-2-en-1-one

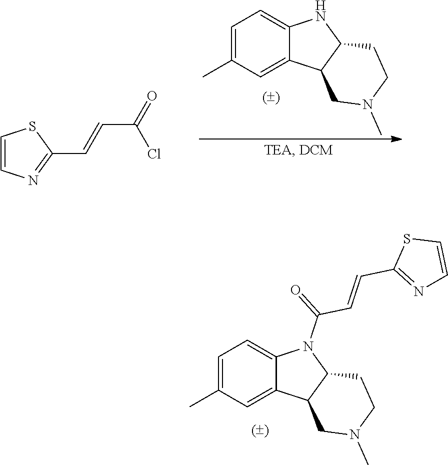 [SMILES: O=C(Cl)/C=C/c1nccs1 ; Cc1ccc2c(c1)[C@@H]1CN(C)CC[C@H]1N2 ; Cc1ccc2c(c1)[C@@H]1CN(C)CC[C@H]1N2C(=O)/C=C/c1nccs1]

To a solution of trans-2,8-dimethyl-2,3,4,4a,5,9b-hexahydro-1H-pyrido[4,3-b]indole (260 mg, 1.3 mmol) in DCM (10 mL) was added TEA (0.53 mL, 3.9 mmol). Then the (E)-3-(thiazol-2-yl)acryloyl chloride (223 mg, 1.3 mmol) in DCM (5 mL) was added dropwise at 0° C. The reaction mixture was stirred at rt overnight, before the mixture was quenched with water (5 mL). The organic phase was dried and concentration. The resulting residue was purified by prep-HPLC to afford the title compound (32.4 mg,7% yield) as a yellow solid. 1H NMR (400 MHz, DMSO-d6) δ 8.00 (d, J=3.2 Hz, 1H), 7.91 (d, J=3.2 Hz, 1H), 7.66 (d, J=15.2 Hz, 1H), 7.37-7.28 (m, 2H), 7.07 (s, 1H), 7.03 (d, J=8.0 Hz, 1H), 3.62-3.56 (m, 1H), 3.46-3.43 (m, 1H), 3.09-3.04 (m, 1H), 3.00-2.97 (m, 1H), 2.67-2.61 (m, 1H), 2.31 (s, 3H), 2.28 (s, 3H), 2.20-2.07 (m, 2H), 2.01-1.92 (m, 1H). MS (ESI) m/z=340.1 [M+H]⁺.

### Example 36: cis-2,5,8-Trimethyl-2,3,4,4a,5,9b-hexahydro-1H-pyrido[4,3-b]indole (CPD-036)

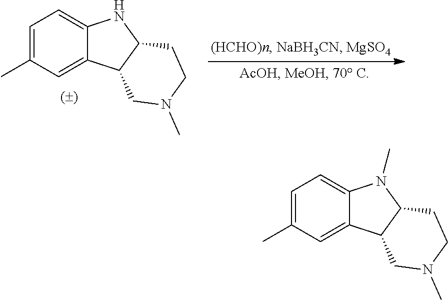 [SMILES: Cc1ccc2c(c1)[C@H]1CN(C)CC[C@H]1N2 ; Cc1ccc2c(c1)[C@H]1CN(C)CC[C@H]1N2C]

CPD-036 was synthesized following the standard procedure for preparing CPD-034 (25 mg, 28% yield) as a colorless oil. ¹H NMR (400 MHz, DMSO-d6) δ 6.86-6.83 (m, 2H), 6.46 (d, J=7.6 Hz, 1H), 3.08-3.01 (m, 2H), 2.65-2.61 (m, 1H), 2.58 (s, 3H), 2.46-2.43 (m, 1H), 2.18 (s, 3H), 2.11 (s, 3H), 2.08-2.02 (m, 1H), 1.92-1.88 (m, 1H), 1.81-1.68 (m, 2H). MS (ESI) m/z=217.2 [M+H]+.

### Example 37: (E)-1-(cis-2,8-Dimethyl-1,2,3,4,4a,9b-hexahydro-5H-pyrido[4,3-b] indol-5-yl)-3-(thiazol-2-yl)prop-2-en-1-one (CPD-037)

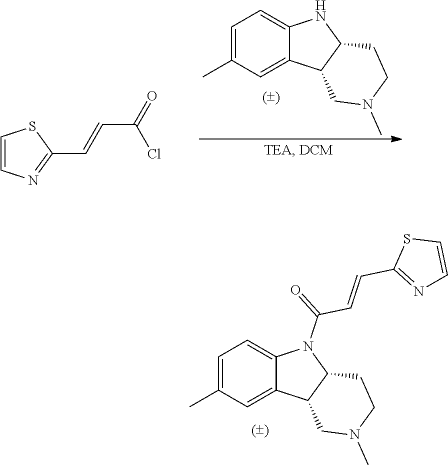 [SMILES: O=C(Cl)/C=C/c1nccs1 ; Cc1ccc2c(c1)[C@H]1CN(C)CC[C@H]1N2 ; Cc1ccc2c(c1)[C@H]1CN(C)CC[C@H]1N2C(=O)/C=C/c1nccs1]

CPD-037 was synthesized following the standard procedure for preparing CPD-035 (35 mg, 58% yield) as a yellow solid. ¹H NMR (400 MHz, DMSO-d6) δ 8.02-8.00 (m, 2H), 7.92 (d, J=2.8 Hz, 1H), 7.78 (d, J=15.2 Hz, 1H), 7.36 (d, J=15.2 Hz, 1H), 7.16 (s, 1H), 7.02 (d, J=8.0 Hz, 1H), 4.80-4.79 (m, 1H), 3.45-3.41 (m, 2H), 2.58-2.56 (m, 1H), 2.33-2.32 (m, 1H), 2.31 (s, 3H), 2.19 (s, 3H), 2.03-1.91 (m, 2H), 1.42-1.34 (m, 1H). MS (ESI) m/z 340.1 [M+H]⁺.

### Example 38: 6-Bromo-2,9-dimethyl-2,3,4,5-tetrahydro-1H-pyrido[4,3-b]indole (CPD-038)

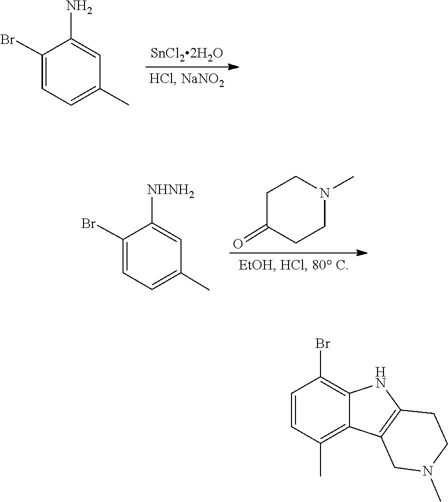

Step 1. Synthesis of (2-bromo-5-methylphenyl)hydrazine

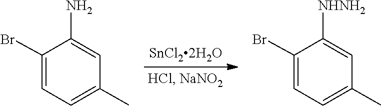

To a solution of 2-bromo-5-methylaniline (9.7 g, 52.14 mmol) in HCl (6M, 160 mL) was added NaNO₂ (3.8 g, 55.1 mmol) at 0° C. After the reaction was stirred at 0° C. for 1.5 h, SnCl₂·2H₂O (16.5 g, 73.0 mmol) in HCl (6M ,100 mL) was added dropwise to the reaction mixture at 0° C. After the resulting mixture was stirred for another 1.5 h at 0° C., the reaction was filtered. The pH value of the filtrate was adjusted to pH=12 with aq. NaOH. The precipitate was collected by filtration, washed with H2O and dried to give the tile compound (8.0 g, 77% yield) as a pale-yellow solid. MS (ESI) m/z=201.1 [M+H]⁺.

Step 2. Synthesis of 6-bromo-2,9-dimethyl-2,3,4,5-tetrahydro-1H-pyrido[4,3-b]indole

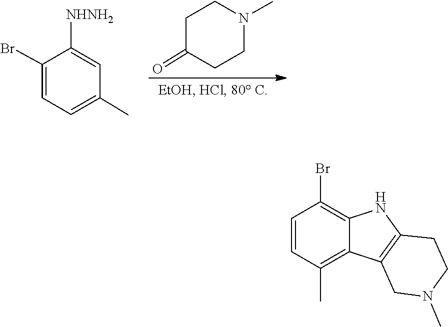

The title compound was synthesized following the standard procedure for preparing CPD-032 (2.0 g, 29% yield) as a yellow solid. ¹H NMR (400 MHz, DMSO-d6) δ 10.87 (s, 1H), 7.04 (d, J=8.0 Hz, 1H), 6.62 (d, J=8.0 Hz, 1H), 3.77 (s, 2H), 2.78 (t, J=4.8 Hz, 2H), 2.69 (t, J=4.8 Hz, 2H), 2.47 (s, 3H), 2.44 (s, 3H). MS (ESI) m/z=279.1 [M+H]⁺.

### Example 39: trans-2,9-Dimethyl-2,3,4,4a,5,9b-hexahydro-1H-pyrido [4,3-b]indole (CPD-039)

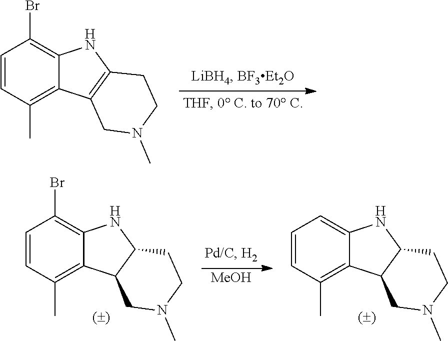 [SMILES: "MeOH" ; Cc1ccc(Br)c2[nH]c3c(c12)CN(C)CC3 ; Cc1ccc(Br)c2c1[C@@H]1CN(C)CC[C@H]1N2 ; Cc1cccc2c1[C@@H]1CN(C)CC[C@H]1N2]

Step 1. Synthesis of trans-6-bromo-2,9-dimethyl-2,3,4,4a,5,9b-hexahydro-1H-pyrido[4,3-b]indole

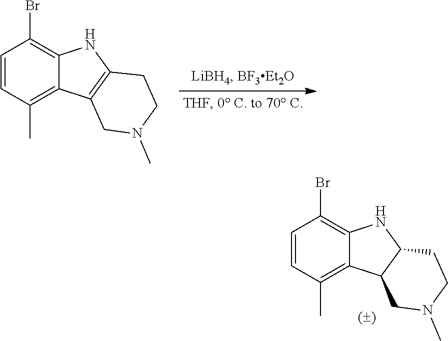 [SMILES: Cc1ccc(Br)c2[nH]c3c(c12)CN(C)CC3 ; Cc1ccc(Br)c2c1[C@@H]1CN(C)CC[C@H]1N2]

To a solution of 6-bromo-2,9-dimethyl-2,3,4,5-tetrahydro-1H-pyrido[4,3-b]indole (500 mg, 1.79 mmol) in THF (10 mL) was added LiBH₄ in THF (2 M, 4.5 mL, 9.0 mmol) at 0° C., followed by BF₃·Et₂O (3.2 mL, 12.5 mmol). The reaction mixture was stirred at rt for 30 min and then at 70° C. for 6 h. HCl (6N, 3.5 mL) was added. The reaction mixture was stirred at 100° C. for 1 h. The pH value was adjusted to pH=10 with NaOH (6 N). After removal of the organic solvent, the residue was extracted with DCM (3×50 mL). The combined organic layers were washed with brine, dried over Na₂SO₄, filtered, concentrated and purified by silica-gel chromatography (DCM/MeOH=20:1 to 10:1) to give the title compound (120 mg, 24%) as white solid. MS (ESI) m/z=281.1 [M+H]⁺.

Step 2. Synthesis of trans-2,9-dimethyl-2,3,4,4a,5,9b-hexahydro-1H-pyrido[4,3-b]indole

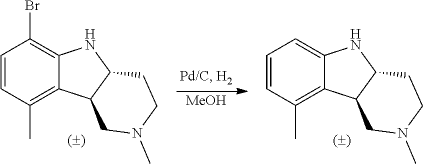 [SMILES: "MeOH" ; Cc1ccc(Br)c2c1[C@@H]1CN(C)CC[C@H]1N2 ; Cc1cccc2c1[C@@H]1CN(C)CC[C@H]1N2]

To a solution of trans-6-bromo-2,9-dimethyl-2,3,4,4a,5,9b-hexahydro-1H-pyrido[4,3-b]indole (500 mg, 1.79 mmol) in MeOH (15 mL) was added Pd/C (40 mg). The resulting mixture was stirred at rt for 4 h under H2. Then the solution was filtered and the filtrate was purified by prep-HPL to give the title compound (50 mg, yield: 14%) as white solid. ¹H NMR (400 MHz, DMSO-d6) δ 6.82 (t, J=7.6 Hz, 1H), 6.43 (d, J=7.6 Hz, 1H), 6.37 (d, J=7.6 Hz, 1H), 5.65 (d, J=2.8 Hz, 1H), 3.53 (dd=10.4, 2.8 Hz, 1H), 2.87-2.81 (m, 2H), 2.70-2.63 (m, 1H), 2.29 (s, 3H), 2.21-2.16 (m, 4H), 2.7-2.00 (m, 1H), 1.93-1.89 (m, 1H), 1.75-1.67 (m, 1H). MS (ESI) m/z=203.1 [M+H]⁺.

### Example 40: trans -2,5,9-Trimethyl-2,3,4,4a,5,9b-hexahydro-1H-pyrido[4,3-b]indole (CPD-040)

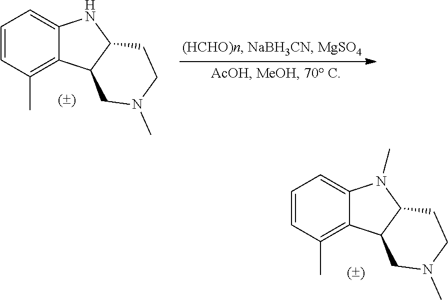 [SMILES: Cc1cccc2c1[C@@H]1CN(C)CC[C@H]1N2 ; Cc1cccc2c1[C@@H]1CN(C)CC[C@H]1N2C]

CPD-040 was synthesized following the standard procedure for preparing CPD-034 (40 mg, 31% yield) as a yellow solid. ¹H NMR (400 MHz, DMSO-d6) δ 6.93 (t, J=8.0 Hz, 1H), 6.46 (d, J=8 Hz, 2H), 3.56 (dd, J=10.4, 3.2 Hz, 1H), 2.94-2.91 (m, 1H), 2.75-2.68 (m, 1H), 2.60 (s, 3H), 2.30 (s, 3H), 2.28-2.22 (m, 1H), 2.19 (s, 3H), 2.18-2.13(m, 1H), 2.06-1.99 (m, 2H), 1.68-1.58 (m, 1H). MS (ESI) m/z=217.2 [M+H]⁺.

### Example 41: cis-2,9-Dimethyl-2,3,4,4a,5,9b-hexahydro-1H-pyrido[4,3-b]indole (CPD-041)

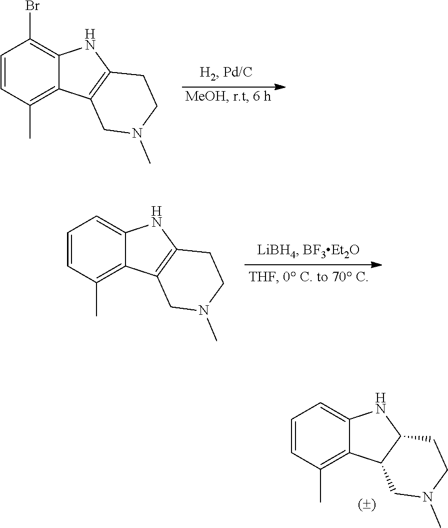

Step 1. Synthesis of 2,9-dimethyl-2,3,4,5-tetrahydro-1H-pyrido[4,3-b]indole

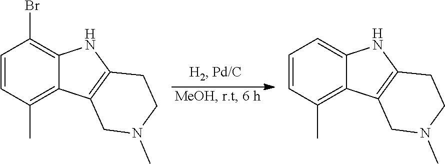

To a solution of 6-bromo-2,9-dimethyl-2,3,4,5-tetrahydro-1H-pyrido[4,3-b]indole (650 mg, 2.33 mmol) in MeOH (15 mL) was added Pd/C (50 mg). After stirring at rt for 4 h under H₂, the reaction mixture was filtered and concentrated to give the tile compound (530 mg, crude) as a yellow solid, which was used directly for the next step. MS (ESI) m/z=203.1 [M+H]⁺.

Step 2. Synthesis of cis-2.9-dimethyl-2,3,4,4a,5,9b-hexahydro-1H-pyrido [4,3-b]indole

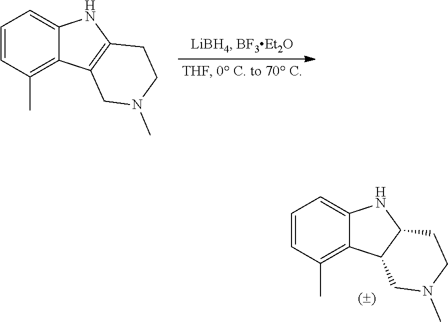

The title compound was synthesized following the standard procedure for preparing CPD-033 (12.2 mg, 3% yield) as white solid. ¹H NMR (400 MHz, DMSO-d6) δ 6.81 (t, J=7.6 Hz, 1H), 6.36 (d, J=7.6 Hz, 2H), 5.38 (d, J=2.4 Hz, 1H), 3.64 (d=2.8 Hz, 1H), 3.10-3.04 (m, 1H), 2.73-2.68 (m, 1H), 2.53-2.51 (m, 1H), 2.15-2.08 (m, 7H), 1.87-1.77 (m, 2H), 1.51 (t, J=11.2 Hz, 1H). MS (ESI) m/z=203.1 [M+H]⁺.

### Example 42: cis -2,5,9-Trimethyl-2,3,4,4a,5,9b-hexahydro-1H-pyrido [4,3-b]indole (CPD-042)

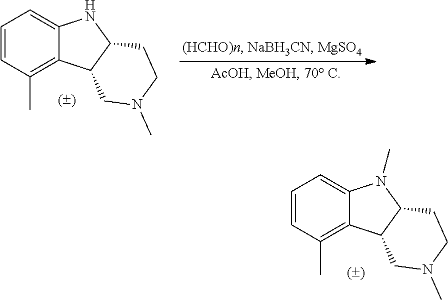 [SMILES: Cc1cccc2c1[C@H]1CN(C)CC[C@H]1N2 ; Cc1cccc2c1[C@H]1CN(C)CC[C@H]1N2C]

CPD-042 was synthesized following the standard procedure for preparing CPD-034 (20 mg, 19% yield) as white solid. ¹H NMR (400 MHz, DMSO-d6) δ 6.94 (t, J=7.6 Hz, 1H), 6.47 (d, J=7.6 Hz, 1H), 6.42 (d, J=7.6 Hz, 1H), 3.20-3.14 (m, 1H), 3.03-3.01 (m, 1H), 2.83-2.79 (m, 1H), 2.64-2.59 (m, 4H), 2.17 (s, 3H), 2.15 (s, 3H), 2.08-2.02 (m, 2H), 1.89-1.78(m, 1H), 1.50 (t, J=11.2 Hz, 1H). MS (ESI) m/z=217.2 [M+H]⁺.

### Example 43: Compound Binding to DCAF1 (FIG. 2A-2F, Table 2).

Binding affinities of compounds to DCAF1 were determined by a surface plasmon resonance (SPR) assay. Purified DCAF1 (1058-1396) proteins were immobilized on a CMS sensor chip, and a dose range of compound solutions were injected in multi-cycle kinetic format. Data were analyzed using a steady state model to provide equivalent dissociation constants (K_{d}). The binding affinities (K_{d} values) of selected compounds are set forth in Table 2. The data showed that some compounds were able to bind DCAF1 in a concentration-dependent manner.

TABLE 2
Binding affinities

| Cmpd. No. | K_{d} |
|---|---|
| CPD-001 | B |
| CPD-002 | D |
| CPD-003 | D |
| CPD-004 | B |
| CPD-005 | B |
| CPD-006 | B |
| CPD-007 | D |
| CPD-008 | B |
| CPD-009 | B |
| CPD-010 | D |
| CPD-011 | B |
| CPD-012 | A |
| CPD-013 | B |
| CPD-014 | B |
| CPD-015 | D |
| CPD-016 | B |
| CPD-017 | B |
| CPD-018 | B |
| CPD-019 | B |
| CPD-020 | D |
| CPD-021 | D |
| CPD-022 | D |
| CPD-023 | C |
| CPD-024 | D |
| CPD-025 | D |
| CPD-026 | D |
| CPD-027 | D |
| CPD-028 | D |
| CPD-029 | D |
| CPD-030 | D |
| CPD-031 | D |
| CPD-032 | D |
| CPD-033 | D |
| CPD-034 | D |
| CPD-035 | D |
| CPD-036 | D |
| CPD-037 | D |
| CPD-038 | D |
| CPD-039 | D |
| CPD-040 | D |
| CPD-041 | D |
| CPD-042 | D |
| CPD-043 | B |
| CPD-044 | B |
| CPD-045 | B |
| CPD-046 | D |
| CPD-047 | D |
| CPD-048 | A |
| CPD-049 | A |
| CPD-050 | C |
| CPD-051 | A |
| CPD-052 | B |
| CPD-053 | B |
| CPD-054 | D |
| CPD-055 | D |
| CPD-056 | A |
| CPD-057 | D |
| CPD-058 | C |
| CPD-059 | B |
| CPD-060 | C |
| CPD-061 | D |
| CPD-062 | D |
| CPD-063 | D |
| CPD-064 | D |
| CPD-065 | A |
| CPD-066 | D |
| CPD-067 | D |
| CPD-068 | D |
| CPD-069 | D |
| CPD-070 | D |
| CPD-071 | D |
| CPD-072 | D |
| CPD-073 | D |
| CPD-074 | D |
| CPD-075 | D |
| CPD-076 | B |
| CPD-077 | D |
| CPD-078 | D |
| CPD-079 | D |
| CPD-080 | D |
| CPD-081 | D |
| CPD-082 | D |
| CPD-083 | D |
| CPD-084 | B |
| CPD-085 | D |
| CPD-086 | D |
| CPD-087 | B |
| CPD-088 | B |
| CPD-089 | C |
| CPD-090 | B |
| CPD-091 | C |
| CPD-092 | B |
| CPD-093 | B |
| CPD-094 | B |
| CPD-095 | B |
| CPD-096 | D |
| CPD-097 | D |
| CPD-098 | B |
| CPD-099 | B |
| CPD-100 | C |
| CPD-101 | D |
| CPD-102 | D |
| CPD-103 | D |
| CPD-104 | C |
| CPD-105 | A |

K_{d} values: A ≤ 40 μM; 40 < B ≤ 70 μM; 70 < C ≤ 100 μM; D > 100 μM

### Materials and Methods

General Chemistry Methods: Chemicals and reagents were purchased from commercial suppliers and used without further purification. LCMS spectra for compounds were acquired using a Waters LC-MS AcQuity H UPLC class system. The Waters LC-MS AcQuity H UPLC class system comprising a pump (Quaternary Solvent Manager) with degasser, an autosampler (FTN), a column oven (40° C., unless otherwise indicated), a photo-diode array PDA detector. Chromatography was performed on an AcQuity UPLC BEH C18 (1.7 μm, 2.1×50 mm) with water containing 0 1% formic acid as solvent A and acetonitrile containing 0.1% formic acid as solvent B at a flow rate of 0.6 mL/min. Flow from the column was split to a MS spectrometer. The MS detector was configured with an electrospray ionization source. Nitrogen was used as the nebulizer gas. Data acquisition was performed with a MassLynx data system. Nuclear Magnetic Resonance spectra were recorded on a Bruker Avance III400 spectrometer. Chemical shifts are expressed in parts per million (ppm) and reported as δ value (chemical shift δ). Coupling constants are reported in units of hertz (J value, Hz; Integration and splitting patterns: where s=singlet, d=double, t=triplet, q=quartet, brs=broad singlet, m=multiple). The purification of intermediates or final products were performed on Agilent Prep 1260 series with UV detector set to 254 nm or 220 nm. Samples were injected onto a Phenomenex Luna C18 column (5 μm, 30×75 mm,) at room temperature. The flow rate was 40 mL/min. A linear gradient was used with either 10% or 50% MeOH in H₂O containing 0.1% TFA as solvent A and 100% of MeOH as solvent B. Alternatively, the products were purified on CombiFlash® NextGen 300 system with UV detector set to 254 nm, 220 nm or 280 nm. The flow rate was 40 mL/min. A linear gradient was used with H₂O containing 0.05% TFA as solvent A and 100% of MeOH containing 0.05% TFA as solvent B. The compounds showed >95% purity using the LCMS methods described herein.

Protein Expression and Purification: Human DCAF1 (1058-1396) (UniPro: Q9Y4B6) coding sequences were cloned into pFastBacHT vector and were expressed in Sf9 cells using Bac-to-Bac baculovirus expression system (Thermo Fisher Scientific). The expression construct for DCAF1 (1058-1396) included a N-terminal His6-tag to facilitate the purification. DCAF1 (1058-1396) proteins were obtained from supernatants of cell lysates and purified through sequential application of Ni affinity chromatography (Ni-NTA column, Bio-Rad), Tag removal using TEV protease, and size-exclusion chromatography (Superdex 200 column, GE Healthcare).

Surface plasmon resonance (SPR) binding assays: SPR studies were performed on a Biacore X100 plus or T200 instrument (GE Healthcare). Immobilization of purified DCAF1 (1058-1396) was carried out at 25° C. using a CMS sensor chip. The surface was pre-equilibrated in HBS-EP running buffer (10 mM HEPES, pH 7.4, 150 mM NaCl, 3 mM EDTA, 0.05% P20), before being activated with EDC/NHS. DCAF1 (1058-1396) was immobilized through amino groups to a density of 9,000-12,000 resonance units (RUs) on flow cell channel 2 (FC2), whereas flow cell channel 1 (FC1) was used as reference. Both DCAF1 immobilized and reference surfaces were deactivated with 1 M ethanolamine.

Interaction experiments were performed at 25° C. The compounds were prepared and serially diluted in HBS-EP running buffer containing final 2% DMSO (6-point two-fold serial dilution, 100 μM -3.125 μM final concentration of compounds). Compound Solutions were injected individually in multi-cycle kinetic format without regeneration (flow rate 30 μL/min, association time 60 s, dissociation time 60 s). Sensorgrams from reference surfaces and blank injections were subtracted from the raw data (double-referenced) and the data was solvent-corrected prior to analysis. Data were analyzed using a steady state affinity model through Biacore Evaluation Software to provide equivalent dissociation constants (K_{d}).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A compound of Formula Ia: 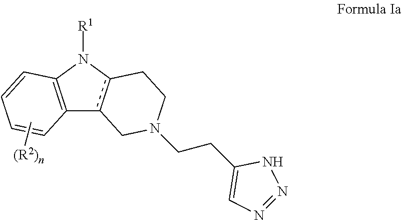
or a pharmaceutically acceptable salt thereof, wherein: [symbol]
is a single bond or a double bond;
R¹ is selected from H, C₁₋₁₀alkyl, C₂₋₁₀alkenyl, C₂₋₁₀alkynyl, C₁₋₁₀haloalkyl, and C₁₋₁₀heteroalkyl;
each R² is independently selected from H, halo, hydroxy, amino, cyano, nitro, C₁₋₈alkyl, C₂₋₈alkenyl, C₂₋₈alkynyl, C₁₋₈haloalkyl, C₁₋₈heteroalkyl, C₁₋₈alkoxy, C₃₋₁₀carbocyclyl, 3- to 10-membered heterocyclyl, C₆₋₁₀aryl, and 5- to 10-membered heteroaryl; and
n is 0, 1, 2, 3, or 4;
provided that the compound of Formula Ia is not

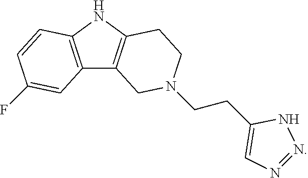 [SMILES: Fc1ccc2[nH]c3c(c2c1)CN(CCc1cnn[nH]1)CC3]

2. A compound selected from: 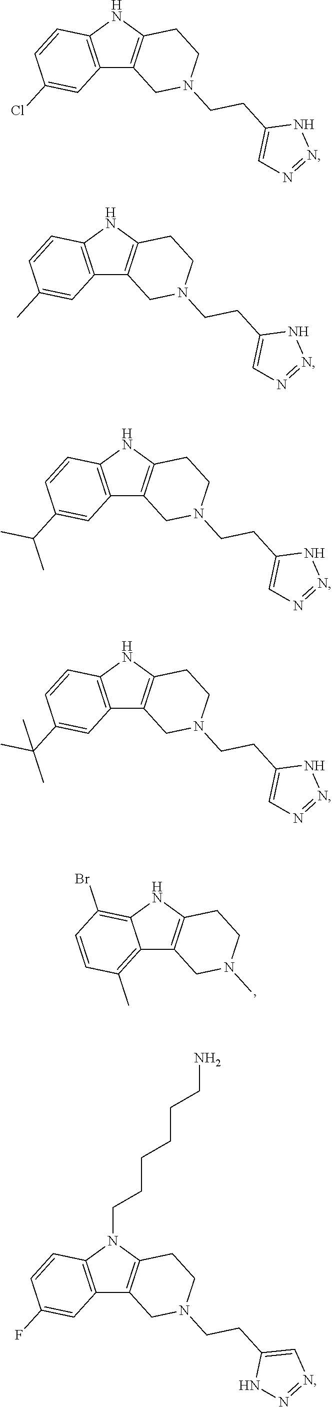 [SMILES: Clc1ccc2[nH]c3c(c2c1)CN(CCc1cnn[nH]1)CC3 ; Cc1ccc2[nH]c3c(c2c1)CN(CCc1cnn[nH]1)CC3 ; CC(C)c1ccc2[nH]c3c(c2c1)CN(CCc1cnn[nH]1)CC3 ; CC(C)(C)c1ccc2[nH]c3c(c2c1)CN(CCc1cnn[nH]1)CC3 ; Cc1ccc(Br)c2[nH]c3c(c12)CN(C)CC3 ; NCCCCCCn1c2c(c3cc(F)ccc31)CN(CCc1cnn[nH]1)CC2] 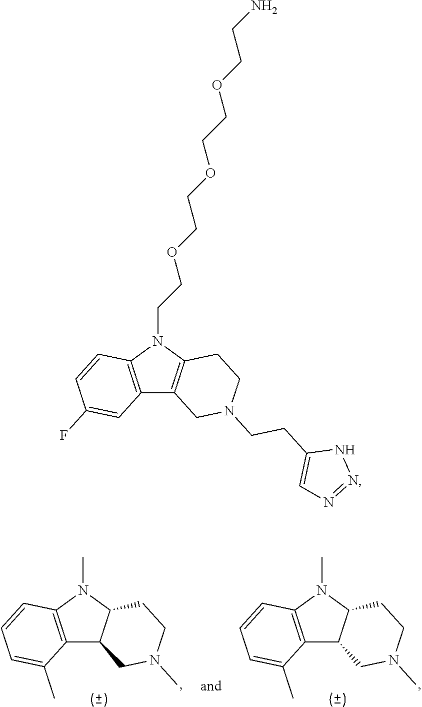
or a pharmaceutically acceptable salt thereof.

3. A method of binding or modulating DDB1- and CUL4-Associated Factor 1 (DCAF1) in a subject in need thereof, comprising administering a therapeutically effective amount of a compound of Formula I: 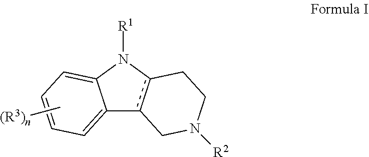
or a pharmaceutically acceptable salt thereof, wherein: [symbol]
is a single bond or a double bond;
R¹ is selected from H, C₁₋₈alkyl, C₂₋₈alkenyl, C₂₋₈alkynyl, C₁₋₈haloalkyl, C₁₋₈heteroalkyl, C₃₋₁₀carbocyclyl, 3- to 10-membered heterocyclyl, C₆₋₁₀aryl, 5- to 10-membered heteroaryl, —C(O)(C₁₋₈alkyl), —C(O)(C₂₋₈alkenyl), —C(O)(C₂₋₈alkynyl), —C(O)(C₁₋₈haloalkyl), —C(O)(C₁₋₈heteroalkyl), —C(O)(C₃₋₁₀carbocyclyl), —C(O)(3- to 10-membered heterocyclyl), —C(O)(C₆₋₁₀aryl), —C(O)(5- to 10-membered heteroaryl), —SO₂(C₁₋₈alkyl), —SO₂(C₂₋₈alkenyl), —SO₂(C₂₋₈alkynyl), —SO₂(C₁₋₈haloalkyl), —SO₂(C₁₋₈heteroalkyl), —SO₂(C₃₋₁₀carbocyclyl), —SO₂(3- to 10-membered heterocyclyl), —SO₂(C₆₋₁₀aryl), and —SO₂(5- to 10-membered heteroaryl), wherein each alkyl, alkenyl, alkynyl, haloalkyl, heteroalkyl, carbocyclyl, heterocyclyl, aryl, and heteroaryl is independently optionally substituted with one or more substituents independently selected from halo, hydroxy, oxo, amino, cyano, nitro, C₁₋₈alkyl, C₂₋₈alkenyl, C₂₋₈alkynyl, C₁₋₈haloalkyl, C₁₋₈heteroalkyl, C₁₋₈alkoxy, C₃₋₁₀carbocyclyl, 3- to 10-membered heterocyclyl, C₆₋₁₀aryl, and 5- to 10-membered heteroaryl;
R² is selected from C₁₋₈alkyl, C₂₋₈alkenyl, C₂₋₈alkynyl, C₁₋₈haloalkyl, C₁₋₈heteroalkyl, C₃₋₁₀carbocyclyl, and 3- to 10-membered heterocyclyl, wherein each alkyl, alkenyl, alkynyl, haloalkyl, heteroalkyl, carbocyclyl, and heterocyclyl, is independently optionally substituted with one or more substituents independently selected from halo, hydroxy, oxo, amino, cyano, nitro, C₁₋₈alkyl, C₂₋₈alkenyl, C₂₋₈alkynyl, C₁₋₈haloalkyl, C₁₋₈heteroalkyl, C₁₋₈alkoxy, C₃₋₁₀carbocyclyl, 3- to 10-membered heterocyclyl, C₆₋₁₀aryl, and 5- to 10-membered heteroaryl;
each R³ is independently selected from halo, hydroxy, amino, cyano, nitro, C₁₋₈alkyl, C₂₋₈alkenyl, C₂₋₈alkynyl, C₁₋₈haloalkyl, C₁₋₈heteroalkyl, C₁₋₈alkoxy, C₃₋₁₀carbocyclyl, 3- to 10-membered heterocyclyl, C₆₋₁₀aryl, and 5- to 10-membered heteroaryl; and
n is 0, 1, 2, 3, or 4.

4. A compound of Formula IIa: 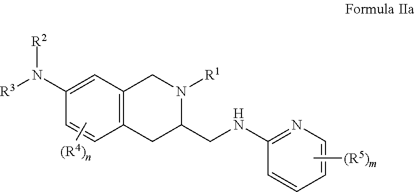
or a pharmaceutically acceptable salt thereof, wherein:
R¹ is selected from H, C₁₋₈alkyl, C₂₋₈alkenyl, C₂₋₈alkynyl, C₁₋₈haloalkyl, and C₁₋₈heteroalkyl;
R² and R³ are independently selected from H, C₁₋₈alkyl, C₂₋₈alkenyl, C₂₋₈alkynyl, C₁₋₈haloalkyl, and C₁₋₈heteroalkyl;
each R⁴ and each R⁵ are independently selected from H, halo, hydroxy, amino, cyano, nitro, C₁₋₈alkyl, C₂₋₈alkenyl, C₂₋₈alkynyl, C₁₋₈haloalkyl, C₁₋₈heteroalkyl, C₁₋₈alkoxy, C₃₋₁₀carbocyclyl, 3- to 10-membered heterocyclyl, C₆₋₁₀aryl, and 5- to 10-membered heteroaryl;
n is 0, 1, 2, or 3; and
m is 0, 1, 2, 3, or 4.

5. A compound selected from: 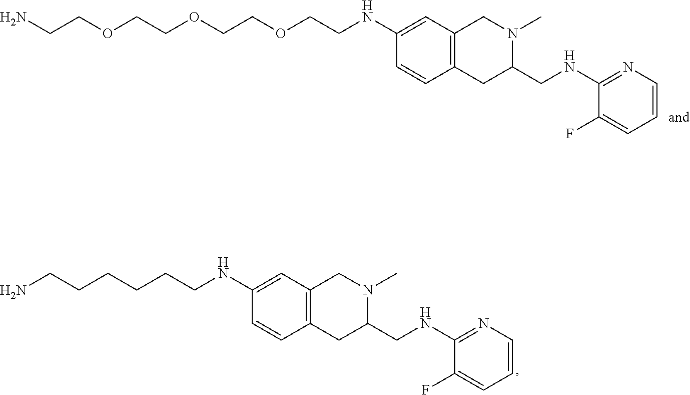 [SMILES: CN1Cc2cc(NCCOCCOCCOCCN)ccc2CC1CNc1ncccc1F ; "and" ; CN1Cc2cc(NCCCCCCN)ccc2CC1CNc1ncccc1F ; ","]
or a pharmaceutically acceptable salt thereof

6. A method of binding or modulating DDB1- and CUL4-Associated Factor 1 (DCAF1) in a subject in need thereof, comprising administering a therapeutically effective amount of a compound of Formula II: 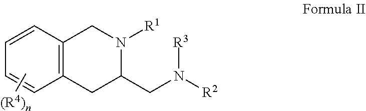
or a pharmaceutically acceptable salt thereof, wherein:
R¹ is selected from H, C₁₋₈alkyl, C₂₋₈alkenyl, C₂₋₈alkynyl, C₁₋₈haloalkyl, and C₁₋₈heteroalkyl;
R² and R³ are independently selected from H, C₁₋₈alkyl, C₂₋₈alkenyl, C₂₋₈alkynyl, C₁₋₈haloalkyl, C₁₋₈heteroalkyl, C₃₋₁₀carbocyclyl, 3- to 10-membered heterocyclyl, C₆₋₁₀aryl, 5- to 10-membered heteroaryl, —C(O)(C₁₋₈alkyl), —C(O)(C₂₋₈alkenyl), —C(O)(C₂₋₈alkynyl), —C(O)(C₁₋₈haloalkyl), —C(O)(C₁₋₈heteroalkyl), —C(O)(C₃₋₁₀carbocyclyl), —C(O)(3- to 10-membered heterocyclyl), —C(O)(C₆₋₁₀aryl), —C(O)(5- to 10-membered heteroaryl), —SO₂(C₁₋₈alkyl), —SO(C₂₋₈alkenyl), —SO₂(C₂₋₈alkynyl), —SO₂(C₁₋₈haloalkyl), —SO₂(C₁₋₈heteroalkyl), —SO₂(C₃₋₁₀carbocyclyl), —SO₂(3- to 10-membered heterocyclyl), —SO₂(C₆₋₁₀aryl), and —SO₂(5- to 10-membered heteroaryl), wherein each alkyl, alkenyl, alkynyl, haloalkyl, heteroalkyl, carbocyclyl, heterocyclyl, aryl, and heteroaryl is independently optionally substituted with one or more substituents independently selected from halo, hydroxy, oxo, amino, cyano, nitro, C₁₋₈alkyl, C₂₋₈alkenyl, C₂₋₈alkynyl, C₁₋₈haloalkyl, C₁₋₈heteroalkyl, C₁₋₈alkoxy, C₃₋₁₀carbocyclyl, 3- to 10-membered heterocyclyl, C₆₋₁₀aryl, and 5- to 10-membered heteroaryl;
each R⁴ is independently selected from halo, hydroxy, amino, cyano, nitro, C₁₋₈alkyl, C₂₋₈lkenyl, C₂₋₈alkynyl, C₁₋₈haloalkyl, C₁₋₈heteroalkyl, C₁₋₈alkoxy, C₃₋₁₀carbocyclyl, 3- to 10-membered heterocyclyl, C₆₋₁₀aryl, and 5- to 10-membered heteroaryl; and
n is 0, 1, 2, 3, or 4.

7. A compound selected from: 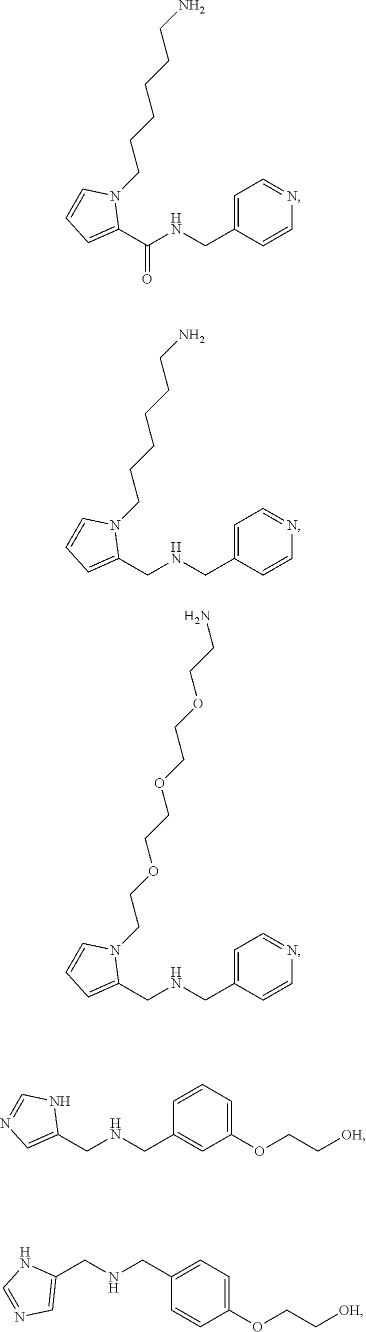 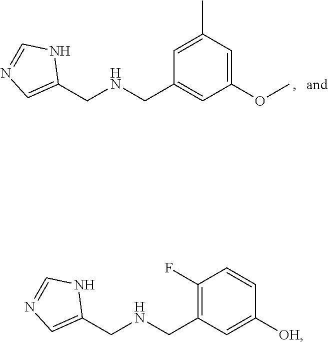 [SMILES: COc1cc(C)cc(CNCc2cnc[nH]2)c1 ; "," ; "and" ; Oc1ccc(F)c(CNCc2cnc[nH]2)c1]
or a pharmaceutically acceptable salt thereof.

8. A method of binding or modulating DDB1- and CUL4-Associated Factor 1 (DCAF1) in a subject in need thereof, comprising administering a therapeutically effective amount of a compound of Formula III: 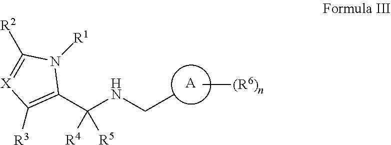
or a pharmaceutically acceptable salt thereof, wherein:
Ring A is selected from null, C₃₋₁₀carbocyclyl, 3- to 10-membered heterocyclyl, C₆₋₁₀aryl, and 5- to 10-membered heteroaryl;
X is selected from N and CR⁷;
R¹ is selected from H, C₁₋₈alkyl, C₂₋₈alkenyl, C₂₋₈alkynyl, C₁₋₈haloalkyl, C₁₋₈heteroalkyl, C₃₋₁₀carbocyclyl, 3- to 10-membered heterocyclyl, C₆₋₁₀aryl, and 5- to 10-membered heteroaryl;
R², R³, and R⁷ are independently selected from H, halo, hydroxy, amino, cyano, nitro, C₁₋₈alkyl, C₂₋₈alkenyl, C₂₋₈alkynyl, C₁₋₈haloalkyl, C₁₋₈heteroalkyl, C₁₋₈alkoxy, C₃₋₁₀carbocyclyl, 3- to 10-membered heterocyclyl, C₆₋₁₀aryl, and 5- to 10-membered heteroaryl;
R⁴ and R⁵ are independently selected from H, halo, cyano, nitro, C₁₋₈alkyl, C₂₋₈alkenyl, C₂₋₈alkynyl, C₁₋₈haloalkyl, C₁₋₈heteroalkyl, C₃₋₁₀carbocyclyl, 3- to 10-membered heterocyclyl, C₆₋₁₀aryl, and 5- to 10-membered heteroaryl; or
R⁴ and R⁵ are taken together to form oxo;
each R⁶ is independently selected from H, halo, hydroxy, amino, cyano, nitro, C₁₋₈alkyl, C₂₋₈alkenyl, C₂₋₈alkynyl, C₁₋₈haloalkyl, C₁₋₈heteroalkyl, C₁₋₈alkoxy, C₃₋₁₀carbocyclyl, 3- to 10-membered heterocyclyl, C₆₋₁₀aryl, and 5- to 10-membered heteroaryl; and
n is 0, 1, 2, 3, 4, 5, or 6.

9. A method comprising contacting the compound of any one of claims 1-8 with a DDB1- and CUL4-associated factor 1 (DCAF1) protein.

10. The method of claim 9, wherein contacting the compound with the DCAF1 protein comprises administering the compound to a cell.

11. The method of claim 9, wherein contacting the compound with the DCAF1 protein comprises administering the compound to a subject.

12. The method of any one of claims 9-11, wherein contacting the compound with the DCAF1 protein comprises contacting the compound with a binding region on the DCAF1 protein, the binding region comprising a WD40 domain.

13. The method of claim 12, wherein the binding region on the DCAF1 protein comprises one or more of the following DCAF1 residues: THR1097, ALA1137, THR1139, HIS1140, THR1155, HIS1180, TYR1181, ARG1225, CYS1227, ILE1262, VAL1265, ARG1298, VAL1299, VAL1300, LYS1327, PRO1329, or PHE1355.

14. The method or compound of any one of claims 1-13, wherein the compound binds DCAF1 non-covalently.

15. The method or compound of any one of claims 1-13, wherein the compound binds DCAF1 with a K_{d}≤40 μM.

16. The method or compound of any one of claims 1-13, wherein the compound binds DCAF1 with a K_{d}>40 and <70 μM.

17. The method or compound of any one of claims 1-13, wherein the compound binds DCAF1 with a K_{d}>70 and <100 μM.

18. The method or compound of any one of claims 1-13, wherein the compound binds DCAF1 with a K_{d}>100 μM.

19. An in vivo modified protein comprising a DDB1- and CUL4-associated factor 1 (DCAF1) protein directly bound to a ligand at a binding region on the DCAF1 protein, the binding region comprising a WD40 domain.

20. The in vivo modified protein of claim 19, wherein the binding region on the DCAF1 protein comprises one or more of the following DCAF1 residues: THR1097, ALA1137, THR1139, HIS1140, THR1155, HIS1180, TYR1181, ARG1225, CYS1227, ILE1262, VAL1265, ARG1298, VAL1299, VAL1300, LYS1327, PRO1329 or PHE1355.

21. The in vivo modified protein of claim 19 or 20, wherein the ligand binds the DCAF1 protein non-covalently.

22. The in vivo modified protein of any one of claims 19-21, wherein the ligand binds the DCAF1 protein with a K_{d}≤40 μM.

23. The in vivo modified protein of any one of claims 19-21, wherein the ligand binds the DCAF1 protein with a K_{d}>40 and ≤70 μM.

24. Th The in vivo modified protein of any one of claims 19-21, wherein the ligand binds the DCAF1 protein with a K_{d}>70 and ≤100 μM.

25. The in vivo modified protein of any one of claims 19-21, wherein the ligand binds the DCAF1 protein with a K_{d}>100 μM.

26. The in vivo modified protein of any one of claims 19-25, wherein the ligand is synthetic.

27. The in vivo modified protein of any one of claims 19-26, wherein the ligand is a small molecule.

28. The in vivo modified protein of any one of claims 19-27, wherein the ligand comprises the compound of any one of claims 1-8.
